(19) United States
(12) Patent Application Publication
(10) Pub. No.: US 2025/0381004 A1
(43) Pub. Date: Dec. 18, 2025

(54) VASCULAR ACCESS ROBOTIC SYSTEMS AND DEVICES INCLUDING CARTRIDGE ASSEMBLIES, AND METHODS THEREOF

(71) Applicant: Hyperion Surgical, Inc., Dania Beach, FL (US)

(72) Inventor: Jonathan AZEVEDO, Fort Lauderdale, FL (US)

(73) Assignee: Hyperion Surgical, Inc., Dania Beach, FL (US)

(21) Appl. No.: 19/246,579

(22) Filed: Jun. 23, 2025

Related U.S. Application Data
(63) Continuation of application No. PCT/US2023/085933, filed on Dec. 26, 2023.
(60) Provisional application No. 63/435,115, filed on Dec. 23, 2022.
(60) Provisional application No. 63/487,834, filed on Mar. 1, 2023.

(51) Int. Cl. A61B 34/00; A61B 17/00; A61B 17/34; A61B 34/20; A61B 34/30; A61B 46/10

(52) U.S. Cl. CPC A61B 34/77; A61B 17/3403; A61B 34/20; A61B 34/25; A61B 34/30; A61B 46/10; A61B 2017/0023; A61B 2017/00477; A61B 2017/00778; A61B 2034/2063; A61B 2034/2074; A61B 2034/303

(57) ABSTRACT

An apparatus for vascular access is described herein. The apparatus can comprise a manipulating device configured to releasably couple a cartridge including a needle, a catheter, and a guidewire that are coaxially disposed with respect to each other, and a robotic arm coupled to the manipulating device. The manipulation device or cartridge can include a plurality of actuation mechanisms configured to selectively advance the needle, the catheter, and the guidewire when the manipulating device is coupled to the cartridge. The robotic arm can include a plurality of joints that are configured to rotate about a plurality of axes to position the cartridge relative to the arm of the patient such that the needle, the catheter, and the guidewire can be inserted into a target vessel of the patient.

### CROSS-REFERENCE TO RELATED APPLICATIONS

This application is a continuation of International Patent Application No. PCT/US2023/085933 filed Dec. 26, 2023, which claims priority to U.S. Provisional Application No. 63/435,115 filed Dec. 23, 2022, and to U.S. Provisional Application No. 63/487,834 filed Mar. 1, 2023, the disclosures of which are incorporated herein in reference in their entirety.

### TECHNICAL FIELD

The present disclosure relates generally to robotic systems, devices, and methods for vascular access. More specifically, the present disclosure relates to robotic systems, devices, and methods for positioning a needle and/or catheter into a blood vessel of a subject.

### BACKGROUND

Interventional medical procedure is a popular procedure that can diagnose and treat diseases in various organs of a patient. Interventional medical procedures are minimally-invasive image guided medical procedures that minimize risk to the patient in comparison to open surgeries.

In order to perform an interventional medical procedure on the vascular system, an operator may need to gain safe access to one or more blood vessels. Traditionally, sharp trocars were used to access blood vessels. Trocars can be used to create lumens through which a catheter can be eventually inserted. However, there are several drawbacks associated with trocars. For instance, improperly placed trocars can lead to various complications such as organ injury, hemorrhage, failed access, catheter malposition, infection, etc.

More recently, the Seldinger technique has been widely employed in order to gain safe access to the vascular system. The Seldinger technique involves puncturing a desired blood vessel with a needle. A guidewire is inserted through the needle such that the guidewire is placed into the blood vessel. Once the guidewire has advanced to the desired length and/or location in the blood vessel, the needle is removed. A catheter is then advanced into the blood vessel over the guidewire. Once the catheter is in the blood vessel, the guidewire is pulled out. The Seldinger technique has fewer complications in comparison to using trocars.

However, the Seldinger technique often requires an experienced surgeon and/or operator to perform the technique on a patient. For instance, human errors made by a surgeon and/or an operator while inserting the guidewire through the needle, or advancing the guidewire to the desired location in the blood vessel can cause complications such as vessel perforation, pseudoaneurysm formation, hemorrhage, infection, etc. Therefore, human errors and inconsistencies while performing the Seldinger technique can cause failures and lead to complications.

Accordingly, there is an unmet need to provide guidance to operators while inserting a needle and/or catheter into a patient to gain safe vascular access and to improve consistency and reduce complications that may arise due to human errors.

### SUMMARY

In some embodiments, an apparatus a cart movable from a first location to a second location near a patient and a manipulation device configured to releasably couple to a cartridge including a needle, a catheter, and a guidewire that are coaxially disposed with respect to each other. The manipulation device and the cartridge include a plurality of actuators each configured to couple to a different one of the needle, the catheter, and the guidewire to selectively advance the needle, the catheter, and the guidewire, when the manipulation device is coupled to the cartridge. The apparatus includes a robotic arm having a first end mounted to the cart and a second end coupled to the manipulation device, the robotic arm having a plurality of segments joined together via a plurality of joints such that the robotic arm can be moved to position the manipulation device.

In some embodiments, an apparatus includes a cartridge. The cartridge includes a guidewire, a needle, and a catheter that are coaxially disposed with respect to each other. The cartridge includes a plurality of guides coupled to the guidewire, the needle, and the catheter. The apparatus includes a manipulation device configured to linearly advance and retract the plurality of guides to move the needle, the guidewire, and the catheter. The apparatus includes a plurality of actuators including at least one actuator disposed in the cartridge and at least one actuator disposed in the manipulation device. The apparatus includes a least one motor and a clutch system configured to operably couple the at least one motor to the at least one actuator disposed in the cartridge.

In some embodiments, a method includes advancing, using a first linear actuator of a manipulation device, a tip of a needle into a target vessel, the needle being coaxially disposed with a catheter and a guidewire in a cartridge selectively coupled to the manipulation device. The method includes advancing, using a second linear actuator of the cartridge, a tip of the guidewire through a lumen of the needle such that the tip of the guidewire extends distally from the tip of the needle. The method includes, after advancing the tip of the guidewire, advancing, using a third linear actuator of the cartridge, a tip of the catheter over at least a portion of the guidewire such that the tip of the catheter extends into the target vessel, the catheter including a catheter hub that is releasable coupled to the third linear actuator via a guide. The method includes after advancing the tip of the catheter, retracting, using the first linear actuator and the second linear actuator, the needle and the guidewire form the target vessel.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a block diagram that illustrates a system for facilitating vascular access, according to some embodiments.

FIG. 2 a block diagram that illustrates a system for facilitating vascular access, according to some embodiments.

FIG. 3 is a block diagram the illustrates a robotic arm of a system for facilitating vascular access, according to some embodiments.

FIG. 4 is a block diagram the illustrates a base of a system for facilitating vascular access, according to some embodiments.

FIG. 5 is a block diagram the illustrates components of a manipulation device and a cartridge assembly and interactions therebetween, according to some embodiments.

FIG. 6 is a flow diagram illustrating a method of performing a vascular access procedure, in accordance with some embodiments.

FIG. 7 is a flow diagram illustrating a method of using a vascular access system to perform the Seldinger technique, in accordance with some embodiments.

FIG. 8 is a flow diagram illustrating a method of using visual aid and/or sensor data to perform a vascular access procedure, in accordance with some embodiments.

FIG. 9 illustrates a vascular access system, in accordance with some embodiments.

FIG. 10A illustrates an example robotic system, in accordance with some embodiments.

FIG. 10B illustrates rotational motion and vertical movement for a base, in accordance with some embodiments.

FIG. 11 illustrates a manipulation device, in accordance with some embodiments.

FIG. 12 illustrates a traverse view and a longitudinal view of a blood vessel as captured by an ultrasound imaging device, in accordance with some embodiments.

FIG. 13 illustrates a cartridge assembly, in accordance with some embodiments.

FIG. 14 illustrates a section view of an area around a catheter of a cartridge assembly, in accordance with some embodiments.

FIG. 15 illustrates a portion of a catheter portion of a cartridge, in accordance with some embodiments.

FIG. 16 illustrates a cartridge with a catheter removed, in accordance with some embodiments.

FIG. 17 illustrates a cartridge with a portion of a housing removed, in accordance with some embodiments.

FIG. 18 illustrates an actuation mechanism, in accordance with some embodiments.

FIG. 19 illustrates an interface system of an actuation mechanism, in accordance with some embodiments.

FIG. 20A illustrates an actuation mechanism with a catheter guide removed, in accordance with some embodiments.

FIG. 20B illustrates an interface system of an actuation mechanism with a catheter guide removed, in accordance with some embodiments.

FIG. 21A illustrates an actuation mechanism with a needle guide removed, in accordance with some embodiments.

FIG. 21B illustrates an interface system of an actuation mechanism with a needle guide removed, in accordance with some embodiments.

FIG. 22 illustrates a cartridge operably coupled to a motor system, in accordance with some embodiments.

FIG. 23 illustrates a motor system, in accordance with some embodiments.

FIG. 24 illustrates an adaptor interfacing between a cartridge and a motor system, in accordance with some embodiments.

FIG. 25 illustrates a clutch system, in accordance with some embodiments.

FIGS. 26A-26E illustrate a clutch system in operation, in accordance with some embodiments.

FIGS. 27A-27E illustrate a spring of a clutch system in operation, in accordance with some embodiments.

FIG. 28 illustrates a translation assembly coupled to a cartridge and a motor system, in accordance with some embodiments.

FIG. 29 illustrates the translation assembly of FIG. 28, in accordance with some embodiments.

FIG. 30 illustrates the anatomical positioning of the manipulation device with respect to a blood vessel, in accordance with some embodiments.

FIG. 31 is a flow diagram illustrating a method of engaging and monitoring a clutch system.

FIG. 32 illustrates an example robotic system, in accordance with some embodiments.

FIG. 33A illustrates a robotic arm of the robotic system of FIG. 32 in a first configuration for storage. FIG. 33B illustrates the robotic arm in a second configuration for vascular access.

FIG. 34A illustrates another example of a cartridge, in accordance with some embodiments.

FIG. 34B illustrates the cartridge of FIG. 34A with internal components shown.

FIG. 35A illustrates a belt actuator, in accordance with some embodiments.

FIG. 35B illustrates a clutch drive, in accordance with some embodiments.

FIG. 36 illustrates an actuator, in accordance with some embodiments

FIG. 37A-37B illustrates another example robotic system, in accordance with some embodiments.

FIG. 38 illustrates a cartridge latch and an adaptor latch, in accordance with some embodiments.

FIGS. 39A-39D illustrate the cartridge latch of FIG. 38 engaging a cartridge release mechanism, in accordance with some embodiments.

FIG. 40 illustrates the adaptor latch of FIG. 38 engaging an adaptor release mechanism, in accordance with some embodiments.

FIGS. 41A-41B depict a clutch system, in accordance with some embodiments.

FIG. 42 is a flow diagram illustrating a method of a homing procedure for a manipulation device, in accordance with some embodiments.

### DETAILED DESCRIPTION

Robotic systems, devices, and methods for vascular access are described herein. In some embodiments, the robotic systems, devices, and methods described herein automate or semi-automate vascular access (e.g., the procedure of the Seldinger technique) in order to provide safe access to blood vessel(s) and/or organ(s). The blood vessel(s) can be any suitable type of blood vessel(s) such as arteries (e.g., radial artery, femoral artery, etc.), veins (e.g., brachial vein, basilic vein, cephalic vein, femoral vein, internal jugular vein, median cubital vein, median antebrachial vein, etc.).

In some embodiments, the technology described herein includes a robotic system for facilitating vascular access. The robotic system can include a manipulation device coupled to a cartridge. The manipulation device and/or the cartridge can comprise or otherwise be attached to a guidewire, a needle, and a catheter that is to be positioned in a blood vessel of a subject. The robotic system and the manipulation device can be controlled by a user (e.g., an operator, a surgeon, etc.) using one or more input/output (I/O) devices. In some embodiments, the manipulation device can include an imaging device (e.g., an ultrasound array). The imaging device can provide the user with visual aid (e.g., ultrasound images of the blood vessel) of the procedure such as the guidewire, the needle, and/or the catheter being inserted into a blood vessel. In some embodiments, the I/O device(s) can include a sensor (e.g., camera) that provides feedback (e.g., image data of the robotic system, manipulation device, and/or portion of a subject's body) to the robotic system as the manipulation device accesses a blood vessel. The robotic system can adjust the movement, position, and/or orientation of the guidewire, needle, and/or catheter based on data from the sensor so as to automate the procedure of vascular access. In some embodiments, the user can remotely control the robotic system and/or the manipulation device to perform the procedure based on the data from the sensor and the visual aid from the imaging device. Further details of such a system are described below with reference to the figures.

### Vascular Access System and Components

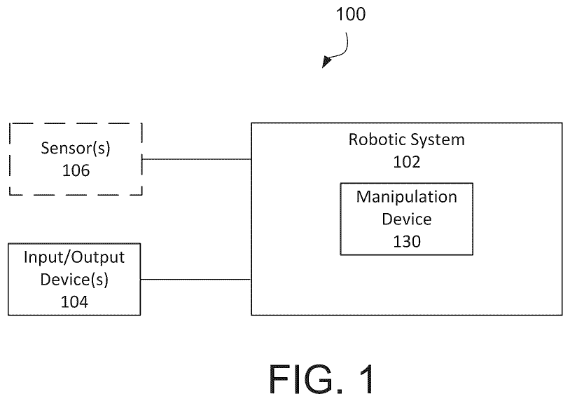

FIG. 1 is a high-level block diagram that illustrates a system 100, according to some embodiments. System 100 can be configured to automate and/or semi-automate a medical procedure for vascular access. System 100 includes a robotic system 102 including a manipulation device 130. The robotic system 102 and/or the manipulation device 130 can be communicably coupled to one or more I/O device(s) 104 (e.g., external and/or remote I/O devices). In some embodiments, the robotic system 102 and/or the manipulation device 130 can be optionally communicably coupled to one or more sensor(s) 106 (e.g., external and/or remote sensors).

In some embodiments, the robotic system 102 can be any suitable robot. For instance, the robotic system 102 can include a robotic arm that can form a part of a robotic device. The robotic device itself can be an autonomous and/or a semi-autonomous cart coupled to and/or integrated with the manipulation device. In some embodiments, the robotic device can include a base with a flat portion that is configured to support a patient on whom the medical procedure is to be performed as further described herein. Alternatively, the robotic device can be an autonomous robot with humanoid features (e.g., arms, transport elements, head, base, etc.).

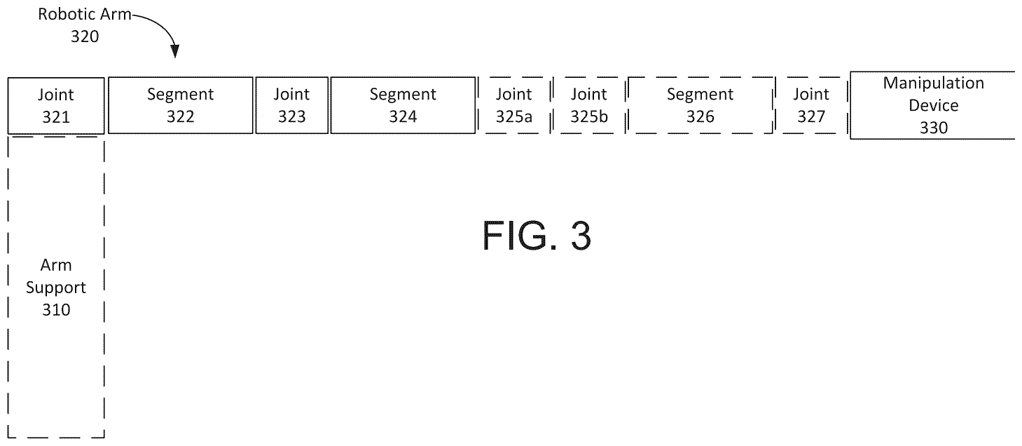

The robotic system 102 can include a robotic arm with two or more segments coupled together via joints, as further detailed with reference to FIG. 3. Joints can allow one or more degrees of freedom. For example, joints can provide for translation along and/or rotation of the robotic arm about one or more axes. In some embodiments, one end segment of the robotic arm can include a coupling element. The coupling element can couple the robotic arm to the manipulation device 130. The other end segment of the robotic arm can be disposed on, affixed to, mounted on, and/or integrated with at least a portion of the robotic system 102.

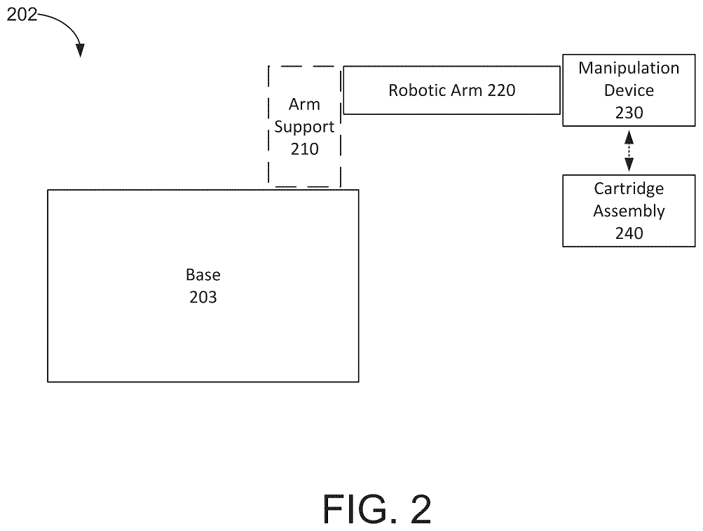

In some embodiments, the robotic arm can be disposed on, affixed to, mounted on, and/or integrated with a base (e.g., base of an autonomous and/or semi-autonomous cart) of the robotic system 102, as further detailed with reference to FIG. 2. In some embodiments, the base can carry the robotic arm, one or more I/O device(s) 104, and one or more sensor(s) 106. The base can be a movable base with one or more transport elements that can provide for translation along and/or rotation of the robotic system 102 along one or more axes. Additionally or alternatively, the base can be configured to be stationary. In some embodiments, the base can be configured to raise vertically so as to position the robotic arm at an appropriate height with respect to a subject (e.g., a patient on whom the medical procedure is to be performed). In some embodiments, the base can include a locking mechanism to lock the movement of the transport elements and/or the movement of the base itself.

In some embodiments, the robotic system 102 can include a communication interface to enable communication with the I/O device(s) 104 and/or the sensor(s) 106. In some embodiments, the robotic system 102 can include a control unit to control the robotic system 102 (e.g., to control the base, robotic arm, etc.).

The robotic system 102 is described as a robotic arm disposed on, affixed to, mounted on, and/or integrated with a base solely for illustrative purposes. It should be readily understood that the robotic system 102 can be any suitable robotic component (e.g., robotic cart, humanoid robot, etc.) that can be coupled to one or more manipulation devices 130. For instance, the robotic system 102 can include multiple robotic arms that form a part of the robotic system 102. Each robotic arm can be coupled to a respective manipulation device. In such a scenario, the robotic system 102 may be configured to perform the medical procedure on multiple subjects substantially simultaneously. Additionally or alternatively, the robotic system 102 may include a robotic arm without a base. Additionally or alternatively, the robotic system 102 can be an autonomous humanoid robot (e.g., a robot with humanoid features such as head, transport elements, manipulation elements, etc.) with a robotic arm for facilitating vascular access.

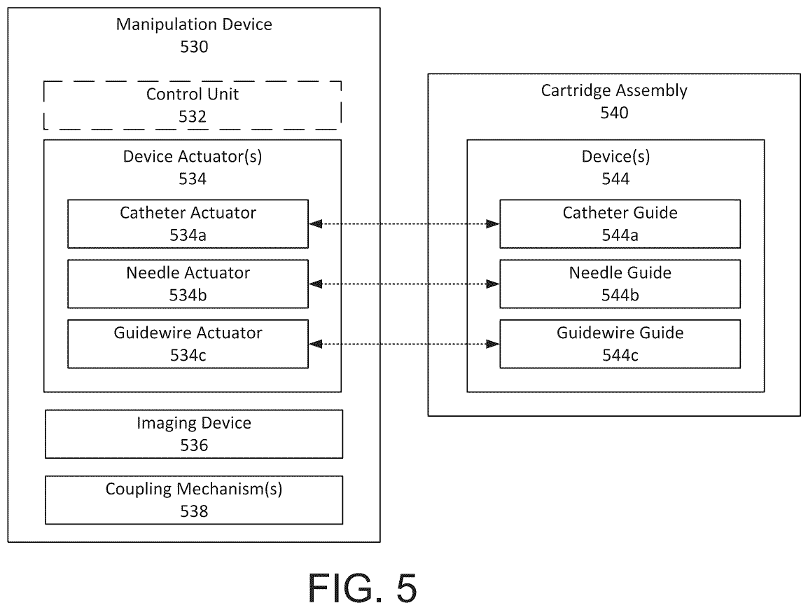

In some embodiments, the manipulation device 130 can be coupled to the robotic system 102 via a coupling element. The manipulation device 130 can be configured to drive movement of one or more components (e.g., a catheter, a needle, and/or a guidewire) to facilitate vascular access. The coupling element can include any type of mechanism that can couple the manipulation device 130 to the robotic system 102, such as, for example, a mechanical mechanism (e.g., a fastener, a latch, a mount, a joint), a magnetic mechanism, a friction fit, etc. The manipulation device 130 can be attached to a cartridge assembly (further described with reference to FIG. 5) that can include a needle, a catheter, and/or a guidewire to perform the medical procedure. In some embodiments, the manipulation device 130 can include one or more actuators that can actuate each of the needle, the catheter, and the guidewire. In some embodiments, one or more actuators are housed within the cartridge assembly. The actuators can enable the manipulation device 130 to perform the medical procedure. The one or more actuators can be any suitable type of actuator. For instance, the one or more actuators can include linear actuators with magnetic encoders.

In some embodiments, the robotic system 102 can include an imaging device (e.g., ultrasound array) to provide a user (e.g., an operator, a surgeon, etc.) with visual aid (e.g., ultrasound images showing traverse view and/or longitudinal view) as the medical procedure is performed (e.g., ultrasound images of the needle, the catheter, and/or the guidewire being inserted into a blood vessel of a subject). In some embodiments, the imaging device can be integrated and/or form part of the manipulation device, as further detailed with reference to FIG. 5.

The manipulation device 130 and/or the robotic system 102 can be communicably coupled to one or more I/O device(s) 104. An I/O device(s) 104 can be any suitable input device that can be configured to receive inputs from the user and/or any suitable output device that can be configured to send outputs to other devices and/or the user operating the robotic system 102. In some embodiments, the I/O device(s) 104 can be an integrated computing device that includes one or more components to both receive inputs and send outputs. Some non-limiting examples of integrated computing device that can receive inputs from the user and send outputs to the user and/or to other devices can include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc.

In some embodiments, the I/O device(s) 104 can be a user control such as a joystick, a remote user control, keyboard, trackball, etc. that can receive input from the user. In some embodiments, the I/O device(s) 104 can be an audio device such as a microphone and/or a speaker that receives audio input from the user. In such embodiments, the I/O device(s) 104 can additionally include a display device (e.g., a display, a touch screen, etc.) that displays output to the user.

The manipulation device 130 and/or the robotic system 102 can be optionally coupled to one or more sensor(s) 106. The sensor(s) 106 can be configured to capture image data of the at least a part of the robotic system 102, the manipulation device 130, and/or at least a part of the subject as the robotic system 102 performs the medical procedure on the subject. The sensor(s) 106 can be an image sensor such as visual camera, stereo camera array, etc. The sensor(s) 106 can be operable to capture two-dimensional and/or three-dimensional images of the robotic system 102, the manipulation device 130, and/or the subject. In some embodiments, the sensor(s) 106 can be operated remotely by the user. For instance, the user can be in a location away from the system 100, and the sensor(s) can be configured to be controlled remotely using one of the I/O device(s) 104. Alternatively, in some embodiments, the user can be in a location proximate to the system 100 and may not require any sensor(s) 106. In some embodiments, a user proximate to the system 100 can also operate and/or adjust one or more sensor(s) 106 of the system 100, e.g., one or more image sensors, to capture views of the environment for one or more remote users and/or for tracking/monitoring purposes.

In some embodiments, the sensor(s) 106 can be mounted on and/or can otherwise be an integral part of the I/O device(s) 104. For instance, the sensor(s) 106 can be attached to, coupled to, and/or otherwise be a part of the I/O device(s) 104. In some embodiments, the sensor(s) 106 can be mounted on the robotic system 102 itself. The sensor(s) 106 can be operable to move (e.g., rotational and/or translational motion) such that the sensor(s) 106 can capture image data from various angles. For instance, the sensor(s) 106 can be mounted on a pan/tilt mechanism to capture the image data. In some embodiments, the sensor(s) 106 can be a portable device such as a handheld computer tablet, a smartphone with camera, or a digital camera that is attached to, mounted on, and/or otherwise a part of the system 100.

In order to perform the medical procedure, the I/O device(s) 104 (e.g., user control such as joystick, keyboard, remote control, trackball, etc.) can receive an input from the user. The input can be transmitted to the robotic system 102 and/or the manipulation device 130. For instance, the I/O device(s) 104 can receive an input to advance the needle, catheter, and/or guidewire into a blood vessel. The input can be transmitted from the I/O device(s) to the robotic system 102 via a communications interface. The robotic system 102 can cause the actuators in the manipulation device 130 to actuate the needle, catheter, and/or guidewire (e.g., included in a cartridge assembly) based on the input. The imaging device (e.g., ultrasound array) included in the manipulation device can provide a visual aid of the movement (e.g., the advancement) of the needle, catheter, and/or guidewire into the blood vessel. The visual aid (e.g., ultrasound images showing traverse view and/or longitudinal view) may be displayed on the I/O device(s) 104 (e.g., display device). Subsequent input representing subsequent movement of the manipulation device 130 or one or more components in the manipulation device 130 (e.g., actuators actuating needle, catheter, and/or guidewire) can be provided to the I/O device(s) 104 based on the visual aid. For example, if the position of the needle, catheter, and/or guidewire in the blood vessel is incorrect, the visual aid (e.g., ultrasound images showing traverse view and/or longitudinal view) can guide the user to modify the input so that such component(s) advance to an appropriate location in the blood vessel.

In some embodiments, the sensor(s) 106 (e.g., camera) can provide image data of the robotic system 102, the manipulation device 130, and the subject to the user. The user can remotely control the manipulation device 130 based on the image data. For example, the image data may include images of the portion of the body of the subject that includes the blood vessel and the orientation and/or position of the manipulation device 130 with respect to the portion of the body. If the orientation and/or position of the manipulation device 130 with respect to the portion of the body is incorrect, the user can remotely control the manipulation device 130 (e.g., by sending instructions to the robotic system 102 via the I/O device(s) 104) so as to orient and/or position the manipulation device 130 as desired.

In some embodiments, the user can control the sensor(s) 106 remotely using the I/O device(s) 104. For instance, if the captured image data does not include images of the manipulation device 130 or the portion of the body, then the sensor(s) 106 can be remotely controlled by the user such that the angle of the sensor(s) 106 can be changed so as to capture the images of both the manipulation device 130 and the portion of the body. For example, the pan/tilt mechanism on which the sensor(s) is mounted can be remotely controlled by the I/O device(s) so as to capture the images as desired.

Subsequent inputs such as input to advance the needle, catheter, and/or guidewire can be provided remotely through the I/O device 104 based on the image data and the visual aid (e.g., ultrasound images) obtained from the imaging device (e.g., ultrasound array). In some embodiments, the robotic system 102 can be configured to automatically (e.g., via the control unit in the robotic system 102) adjust the position and/or orientation of the manipulation device 130 or one or more components of the manipulation device 130 based on the image data and the visual aid. In this manner, the robotic system 102 along with the manipulation device 130 can perform the vascular access procedure (e.g., the Seldinger technique) in an automated and/or a semi-automated manner such as with the user controlling the I/O device(s) 104 that in turn controls and actuates the robotic system 102 and/or the manipulation device 130.

### 1.0 Robotic System

FIG. 2 is a block diagram that illustrates a robotic system 202 of a vascular access system, according to some embodiments. The robotic system 202 can be functionally and/or structurally similar to other robotic systems described herein, such as, for example, robotic system 102 in FIG. 1. The robotic system 202 can include a base 203. The base 203 can be mechanically coupled to a robotic arm 220 (e.g., similar to the robotic arm as described with reference to FIG. 1) via an arm support 210. The robotic arm 220 can be coupled to and/or integrated with a manipulation device 230. In some embodiments, a cartridge assembly 240 can be attached to the manipulation device 230. In some embodiments, the cartridge assembly 240 may be a cartridge configured to access a radial vein. In some embodiments, the cartridge assembly may be configured to access a jugular vein.

### 1.1 Base

The base 203 can be any suitable base for positioning a manipulation device 230 of the vascular access system. For example, the base 203 can be a chassis supporting the robotic arm 220 and the manipulation device 230. In such scenarios, one or more electronic components such as a control unit, a communications interface, etc. can be attached to and/or coupled to the base 203 (e.g., chassis). Alternatively, the base 203 can be a structure supporting the robotic arm 220 and the manipulation device 230 that houses one or more electronic components such as a control unit, a communications interface, etc. within the base 203. Put differently, the outer structure of the base 203 can be a housing that encloses one or more electronic components. The robotic arm 220 and the manipulation device 230 can be supported on the outer structure. In some embodiments, the base 203 can be a surface with a flat portion configured to support a patient on whom the medical procedure is to be performed. For example, the base 203 can be a bed configured to support the patient. Additionally or alternatively, the base 203 can be a platform configured to support the patient. A first portion of the robotic arm 220 can be coupled to the base 203 (e.g., bed, chassis, etc.). A second portion of the robotic arm (e.g., a second portion opposite the first portion) can be coupled to the manipulation device 230. In some embodiments, the second portion of the robotic arm 220 coupled to the manipulation device 230 can be movable relative to the base 203 to position the needle, the guidewire, and the catheter for insertion into the target vessel of the patient.

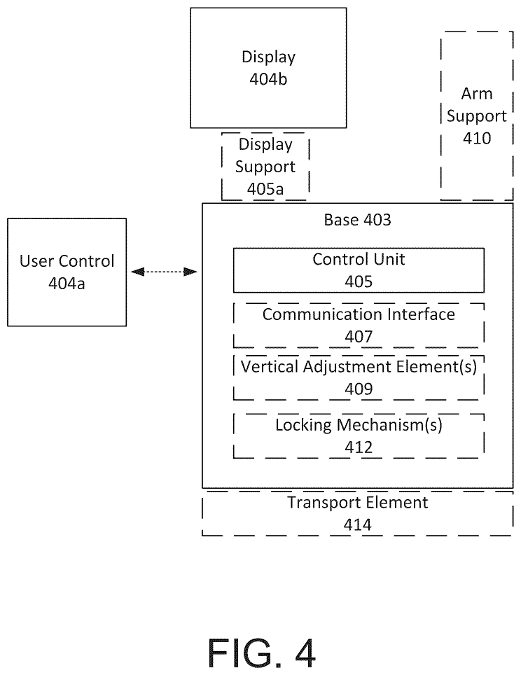

FIG. 4 is a block diagram the illustrates a base 403 (e.g., structurally and/or functionally similar to base 203 in FIG. 2 and/or other bases described herein), according to some embodiments. In some embodiments, the bottom surface of the base 403 can include transport elements 414 that can provide for translation along and/or rotation of the robotic system (e.g., robotic system 202 in FIG. 2) along one or more axes. Transport elements 414 can be any suitable components configured for movement such as, for example, a wheel, a swivel caster, a track, etc. Transport elements can enable the robotic system 202 to move around.

For instance, the transport elements 414 can be swivel casters (e.g., 4 swivel casters coupled to 4 corners of the base 403) that provide three degrees of freedom to the robotic system 202. The swivel casters can allow for linear translations of the robotic system 202 along two axes and rotation of the robotic system 202 along one axis. These three degrees of freedom can enable a user (e.g., a surgeon and/or an operator) to achieve planar and rotational positioning of the base 403 and thereby planar and rotational positioning of the robotic system 202 relative to a portion of a subject's body (e.g., arm, etc., on which the medical procedure is to be performed).

In some embodiments, the base 403 can include vertical adjustment elements 409 to move (e.g., raise or drop) the base vertically so as to position the robotic arm 220 and the manipulation device 230 at an appropriate height with respect to a subject (e.g., a patient on whom the medical procedure is to be performed). This can provide the robotic system 202 with a fourth degree of freedom. In some embodiments, the vertical adjustment elements 409 can include mechanical features to lift a top surface of the base 403 and/or drop the top surface of the base 403 to a specific height. For example, the vertical adjustment elements 409 can include linear rails with recirculating balls to adjust a height of the top surface of the base 403. In some embodiments, the vertical adjustment elements 409 can include an actuator such as ball screw actuator to move the base vertically. For instance, the ball screw actuator can move the top surface of the base 403 vertically to a desired height. Once the desired height is reached, a fail-safe brake can hold the position of the top surface while the linear rails with recirculating balls can constraint the movement of the base 403. In other embodiments, the top surface of the base 403 can be moved manually by manually adjusting the linear rails and the recirculating balls.

In some embodiments, the base 403 can include a locking mechanism 412 to lock the movement of the base 403. For instance, once a user positions the robotic system 202 at an appropriate position (e.g., distance and/or height) with respect to the subject, the locking mechanism can be engaged to lock the position of the base 403 and the robotic system 202. The locking mechanism 412 can lock the transport elements 414 (e.g., swivel casters) preventing the transport elements 414 from moving further. In some embodiments, the locking mechanism 412 can automatically engage a lock. For instance, the locking mechanism 412 can automatically lock the transport elements 414 as soon as the robotic system 202 is positioned at a desired location.

In some embodiments, the base 403 can include a communications interface 407. The communication interface 407 can be any suitable component that enables the base 403 and/or the robotic system 202 to communicate with I/O device(s) (e.g., I/O device(s) 104 in FIG. 1), sensor(s) (e.g., sensor(s) 106 in FIG. 1), or other suitable devices. In some embodiments, communication interface 407 can further enable the I/O device(s) to communicate with the transport elements 414, vertical adjustment elements 409, and locking mechanism 412. In some embodiments, the I/O device(s) can include a user control 404a and/or a display 404b, as further detailed below.

In some embodiments, the base 403 can include a control unit 405 to control and/or monitor one or more components of the robotic system (e.g., robotic system 202 in FIG. 2) such as the base 403, the robotic arm (e.g., robotic arm 220 in FIG. 2), the manipulation device (e.g., manipulation device 230 in FIG. 2), the cartridge assembly (e.g., cartridge assembly 240 in FIG. 2), and/or a combination thereof. Control unit 405 can be any suitable processing device configured to run and/or execute functions associated with controlling and/or monitoring one or more components of the robotic system. Control unit 405 can include any suitable processor(s) that can be configured to execute modules, functions, and/or processes. In some embodiments, the processor(s) can be a general purpose processor, a Field Programmable Gate Array (FPGA), an Application Specific Integrated Circuit (ASIC), a Digital Signal Processor (DSP), and/or the like. In some embodiments, the control unit 405 may be communicatively coupled to or include a sensor (e.g., torque sensor, pressure sensors, ammeters, etc.) configured to monitor the operations of the robotic system.

As discussed above, the base 403 can support the robotic arm (e.g., robotic arm 220 in FIG. 2) and/or one or more I/O device(s) (e.g., I/O device(s) 104 in FIG. 1). For example, the base 403 can be coupled to the robotic arm via arm support 410. One or more I/O device(s) such as, for example, user control 404a and display 404b can be communicably coupled to the base 403. In some embodiments, the display 404b can additionally be mechanically coupled to the base 403 via the display support 405a. Display support 405a can be any suitable support that can attach and/or couple display 404b to the base 403 such that the base 403 supports the display 404b. In some embodiments, display support 405a can enable adjustment(s) to be made to the position and/or orientation of the display 404a. Display 404b can be any suitable display device such as touch screen, device displaying a graphical user interface, audio device (e.g., microphone, speaker, etc.), a combination thereof, and/or the like.

In some embodiments, the user control 404a can additionally be attached to and/or integrated with the base 403. For instance, user control 404a can be integrated with the base 403 such that the base 403 supports the user control 404a. User control 404a can be any suitable device that can receive input from the user such as a joystick, a remote user control, keyboard, trackball, etc.

### 1.2 Robotic Arm

Referring back to FIG. 2, the base 203 can be coupled to the robotic arm 220 via an arm support 210. The robotic arm 220 can comprise of two or more segments coupled together via joints. One end segment can be coupled via a joint to the arm support 210. The other end segment can be integrated with and/or coupled to the manipulation device 230. In some embodiments, the robotic arm 220 can be actuated by one or more motors. In some embodiments, the robotic arm 220 can include one or more sensors to measure sensory information, including information relating to the robotic arm 220. Examples of sensors include position encoders, torque and/or force sensors, touch and/or tactile sensors, etc. The sensors can be disposed on or integrated with either the segments, or the joints, or a combination of both. The sensory information can be transmitted to a control unit (e.g., control unit 405 in FIG. 4) included in or attached to the base 203. Additionally or alternatively, the sensory information can be transmitted to one or more I/O device(s) (e.g., I/O device(s) 104 in FIG. 1).

FIG. 3 is a block diagram the illustrates a robotic arm 320 (e.g., structurally and/or functionally similar to robotic arm 220 in FIG. 2 and/or other robotic arms described herein), according to some embodiments. In some embodiments, robotic arm 320 can be implemented as an arm that includes two segments 322 and 324. Arm support 310 (e.g., similar to arm support 210 in FIG. 2) and segment 322 can be coupled together via joint 321. Segments 322 and 324 are coupled together via joint 323. In some embodiments, the robotic arm can optionally include segment 326. Segments 326 and segment 324 can be coupled together via joints 325a and 325b. In some embodiments, the manipulation device 330 (e.g., manipulation device 230 in FIG. 2) and segment 326 can be coupled together via joint 327. In other embodiments (e.g., embodiments that may not include segment 326), the manipulation device 330 and segment 324 can be coupled together via joint 325a. In yet other embodiments, the manipulation device 330 can be integrated with segment 324 or segment 326.

In some embodiments, the robotic arm 320 can have three proximal axes. A first proximal axis can be along arm support 310 that enables vertical translation of the robotic arm 320 along the arm support 310. A second proximal axis can be along joint 321. A third proximal axis can be along joint 323. The three proximal axes can allow translation of the robotic arm 320 along the three-dimensional space. In some embodiments, the robotic arm can have three distal axes. A first distal axis can be along joint 325a, a second distal axis can be along joint 325b, and a third distal axis can be along joint 327. The three distal axes can allow rotation of the robotic arm 320 along the three-dimensional space (e.g., pitch, yaw, and roll). In this manner, the robotic arm can have six degrees of freedom. In some embodiments, the second proximal axis along joint 321, the third proximal axis along joint 323, and the first distal axis along 325a can comprise a planar Selective Compliance Articulating Robot Arm (SCARA) linkage. While three segments and five joints are depicted in FIG. 3, one of ordinary skill in the art would understand that a robotic arm can include a different number of segments and/or joints.

In some embodiments, the robotic arm 320 can include locking mechanisms for locking one or more components of the robotic arm 320. For example, the robotic arm can include one or more pulleys, magnets, etc. for locking one or more joints and/or a height of the robotic arm 320 relative to a base of a robotic system (e.g., base 403).

### 2.0 Manipulation Device and Cartridge Assembly

Referring back to FIG. 2, an end segment of the robotic arm 220 can be coupled to the manipulation device 230. The manipulation device 230 can be attached to a cartridge assembly 240. Further details of the components of an example manipulation device and an example cartridge assembly are described below.

FIG. 5 is a block diagram that illustrates a manipulation device 530 (e.g., similar to manipulation device 230 in FIG. 2 and/or other manipulation devices described herein) and a cartridge assembly 540 (e.g., similar to cartridge assembly in FIG. 2 and/or other cartridge assemblies described herein), according to some embodiments. In some embodiments, the manipulation device 530 can include a coupling mechanism 538, an imaging device 536, a portion of one or more device actuator(s) (e.g., catheter actuator 534a, needle actuator 534b, and guidewire actuator 534c), collectively referred to as device actuator(s) 534, and optionally a control unit 532. In some embodiments, one or more of the device actuator(s) 534 may be housed within the cartridge assembly 540. For example, the catheter actuator 534a and the guidewire actuator 534c may be located within the cartridge assembly 540 while the needle actuator 534b is located within the manipulation device 530. Any suitable permutation of the catheter actuator 534a, needle actuator 534b, and the guidewire actuator 534c in the manipulation device 530 and/or the cartridge assembly 540 can be possible.

In some embodiments, the cartridge assembly 540 can include the device(s) 544 such as a catheter, a needle, and/or a guidewire. Alternatively, the manipulation device 530 can include some of the device(s) 544 while the cartridge assembly 540 can include other device(s) 544. For instance, the manipulation device 530 can include a catheter and a guidewire while the cartridge assembly 540 can include the needle. Similarly, the manipulation device 530 can include the guidewire and the needle while the cartridge assembly 540 can include the catheter. In a similar manner, any suitable permutation of the catheter, the needle, and the guidewire in the manipulation device 530 and/or the cartridge assembly 540 can be possible. In some embodiments, the guidewire, the needle, and the catheter can be arranged coaxially in the manipulation device 530. For example, the guidewire can be disposed within a lumen of the needle and the needle can be disposed within a lumen of the catheter. In some embodiments, a length of the catheter can be about 40 mm. In some embodiments, a length of the needle can be a little more than 40 mm (40 mm plus bevel length) such that the needle can extend past the catheter. In some embodiments, the guidewire can be 142 mm long such that at least 50 mm of the guidewire can extend past the needle tip. In some embodiments, the cartridge assembly 540 can be configured to store the guidewire in a linear state. In some embodiments, the cartridge assembly 540 can also include another portion of the one or more device actuator(s) (e.g., catheter actuator 534a, needle actuator 534b, and guidewire actuator 534c), collectively referred to as device actuator(s) 534.

The imaging device 536 in the manipulation device 530 can provide the user with a visual aid of a blood vessel as the medical procedure is being performed. For example, the imaging device can be any suitable imaging device that can capture a visual representation of the blood vessel. Some non-limiting example of the imaging device 536 can include ultrasound imaging device, fluoroscopes, cameras, etc.

In some embodiments, the imaging device 536 can be an ultrasound array located on the manipulation device 230. The ultrasound array can provide two-dimensional ultrasound images along a longitudinal plane and a transverse plane. The ultrasound images with the transverse view of a blood vessel can show the radial cross section of the blood vessel and the longitudinal view of the blood vessel can show the axial cross section of the blood vessel. In some embodiments, the imaging device 536 can be configured to obtain three-dimensional ultrasound images of the blood vessel.

The manipulation device 530 and the cartridge assembly 540 can each include a portion of one or more device actuator(s) 534. The device actuator(s) 534 can be configured to actuate the needle, the catheter, and/or the guidewire. For example, the manipulation device 530 can include linear actuators to actuate the device(s) 544. The linear actuators can include a ball screw shaft supported by ball screw bearings. A motor can be coupled to each device actuator 534 to drive the movement of a ball screw nut along the shaft. A magnetic encoder coupled to the motor can sinusoidally commutate the motor. A linear circulating ball bearing can be coupled to the ball screw nut that is fixed on the ball screw shaft. For instance, the linear circulating ball bearing can be coupled to the ball screw nut on the ball screw shaft via a carriage block. As the ball screw shaft rotates (e.g., owing to the rotation of the motor's rotor), the ball screw nut translates as it is constrained by the linear circulating ball bearing through the carriage block. The translation of the ball screw nut can in turn actuate a device(s) 544 along a linear axis. Accordingly, each of the needle, catheter, and guidewire can be actuated along a linear axis by a respective linear actuator. In some embodiments, a portion of the one or more device actuator(s) 534 are located in the manipulation device 530 and a corresponding portion of the one of more device actuator(s) 534 are located in the cartridge assembly 540. For example, a motor of a linear actuator may be located in the manipulation device 530 and a corresponding ball screw may be located in the cartridge assembly 540 and operably coupled to the motor.

Each of the needle, catheter, and guidewire can be attached to a respective guide that guides the device(s) 544 along the linear axis as the device(s) are being actuated by the linear actuators (e.g., device actuator(s) 534 included in manipulation device 530). Therefore, the guides form another portion of the one or more device actuator(s) 534. In some embodiments, the guides can be included in the cartridge assembly 540 and can be attached to the respective device(s) 544. For example, a needle guide 544b included in the cartridge assembly 544 can be attached to the needle, a catheter guide 544a included in the cartridge assembly 544 can be attached to the catheter, and a guidewire guide 544c included in the cartridge assembly 544 can be attached to the guidewire. In some embodiments, the catheter guide 544a, the needle guide 544b, and the guidewire guide 544c can each include a coupling element that can couple with the coupling mechanism 538 in the manipulation device 530.

The manipulation device 530 can include coupling mechanism 538 that couples the cartridge assembly 540 (e.g., the coupling element in the cartridge assembly 540) to the manipulation device 530, such as, for example, a mechanical mechanism (e.g., a fastener, a latch, a mount, clip, etc.), a magnetic mechanism, a friction fit, etc. In some embodiments, the manipulation device 530 and the cartridge assembly 540 are operably coupled to allow for at least a portion of the device actuator(s) 534 in the manipulation device 530 to interface with corresponding portions of device actuator(s) 534 in the cartridge assembly. For example, the manipulation device 530 may be coupled to the cartridge assembly 540 such that motors in the manipulation device 530 may operate ball screws in the cartridge assembly 540 which may operate device(s) 544. In some embodiments, the cartridge assembly 540 is coupled to the manipulation device 530 such that device actuator(s) 534 in the manipulation device 530 may translate the cartridge assembly along the manipulation device 530.

In some embodiments, the coupling mechanism 538 can be a mechanical mechanism. For example, the coupling mechanism 538 can include slots (e.g., recessed portions in the manipulation device 530) such that the catheter guide 544a, the needle guide 544b, and the guidewire guide 544c included in the cartridge assembly 540 can fit within the slots. In some embodiments, the coupling mechanism 538 can be a combination of the magnetic mechanism and the mechanical mechanism.

In some embodiments, the coupling mechanism 538 includes an adaptor for facilitating coupling via the manipulation device 530 and the cartridge assembly 540. The adaptor may be configured to allow for the manipulation device 530 to be isolated (e.g., from potential contaminants, etc.) during operation. The coupling mechanism 538 may include a clutch mechanism between the cartridge assembly 540 and the manipulation device 530 that provides mechanical coupling while allowing for the manipulation device 530 to be isolated.

In some embodiments, the manipulation device 530 can optionally include a control unit 532 to control the actuation of the device actuator(s) 534. Control unit 532 can be any suitable processing device configured to run and/or execute functions associated with controlling the device actuator(s) 534. Control unit 532 can include any suitable processor(s) that can be configured to execute modules, functions, and/or processes. In some embodiments, the processor(s) can be a general purpose processor, a Field Programmable Gate Array (FPGA), an Application Specific Integrated Circuit (ASIC), a Digital Signal Processor (DSP), and/or the like.

### Methods

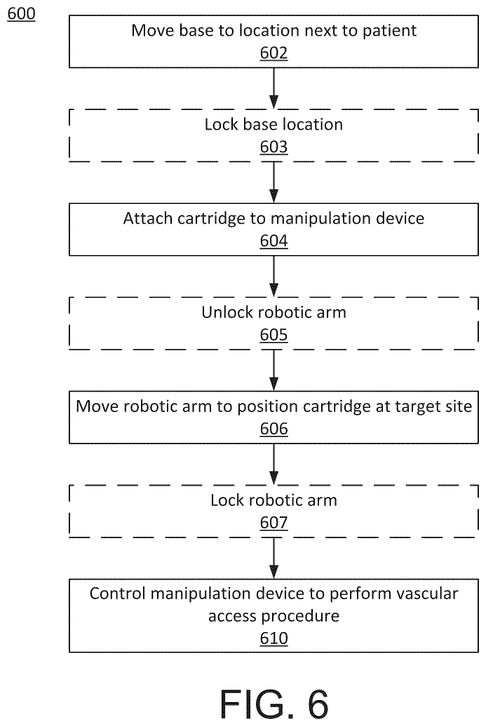

FIG. 6 is a flow diagram illustrating a method 600 of performing a medical procedure (e.g., using system 100 in FIG. 1), in accordance with some embodiments. A robotic system, such as, for example, robotic system 102 in FIG. 1 and/or any of the other robotic systems described herein, can perform the medical procedure (e.g., vascular access procedure) in an automated and/or semi-automated manner. At 602, a base (e.g., base 203 in FIG. 2 or base 403 in FIG. 4) can be moved to a location next to a subject (e.g., a patient). As discussed above, the base can include transport elements (e.g., swivel casters) that can allow for translational and rotational movement of the robotic system. The base can be moved to a suitable position from the subject such that the medical procedure can be performed on the subject. For example, the base can be moved to a position that allows a robotic arm (e.g., robotic arm 220 in FIG. 2 or robotic arm 320 in FIG. 3) access a portion of the subject's body (e.g., patient's arm on which the medical procedure is to be performed). In some embodiments, the base can be moved to position the robotic arm such that at least a portion of the manipulation device and/or the cartridge assembly touches the skin of the subject. In some embodiments, an I/O device (e.g., I/O device(s) 104 in FIG. 1 or user control 404a in FIG. 4) communicably coupled to the base and/or communicably coupled to the transport elements included in the base can transmit instructions to move the base. For example, the I/O device can be configured to transmit instructions to an actuator controlling the movements of the swivel caster. In other embodiments, the base can be moved manually by a user.

In some embodiments, the base can be moved up or dropped down in a vertical manner so as to position the base and/or the robotic arm suitably in order to perform the medical procedure. As discussed above, the base can include vertical adjustment element(s) (e.g., vertical adjustment element(s) 409 in FIG. 4) to adjust a height of a top surface of the base. In some embodiments, an I/O device (e.g., I/O device(s) 104 in FIG. 1 or user control 404a in FIG. 4) communicably coupled to the base and/or communicably coupled to the vertical adjustment element(s) can transmit instructions to adjust the height of the top surface of the base. For example, the I/O device can be configured to transmit instructions to an actuator controlling the vertical adjustment element(s). In other embodiments, the vertical adjustment element(s) can be manipulated manually by a user so as to adjust the height of the top surface of the base. Once the base is moved to a suitable location from the subject (e.g., suitable distance and/or suitable height), the base can be locked (e.g., using locking mechanism(s) 412 in FIG. 4) to lock the location of the base (at 603 in FIG. 6).

At 604, a cartridge assembly (e.g., cartridge assembly 240 in FIG. 2 or cartridge assembly 540 in FIG. 5) can be attached to a manipulation device (e.g., manipulation device 130 in FIG. 1, manipulation device 230 in FIG. 2, manipulation device 330 in FIG. 3, or manipulation device 530 in FIG. 5). The manipulation device can be coupled to or otherwise be a part of the robotic arm. In some embodiments, the manipulation device can include coupling mechanism(s) (e.g., coupling mechanism(s) 538 in FIG. 5) to couple the cartridge assembly to the manipulation device. For example, the manipulation device can include a permanent electromagnet that magnetically couples the manipulation device to the cartridge assembly. A coupling element (e.g., puck) in the cartridge assembly can comprise a magnetic portion (e.g., an embedded stainless-steel disk) that can close a magnetic circuit with the magnet (e.g., permanent electromagnet). The coupling mechanism(s) may include mechanical components to couple the cartridge assembly to the manipulation device. A coupling element (e.g., clutch) in the cartridge assembly can comprise components that allow for mechanical power transfer between the manipulation device and the cartridge assembly. In this manner, the cartridge assembly can be attached to the manipulation device. Additionally or alternatively, the manipulation device can include recessed portions (e.g., slots) to receive portions of the cartridge assembly. For instance, the device(s) (e.g., device(s) 544 in FIG. 5) along with guide(s) (e.g., device actuator(s) 534 in FIG. 5) can be configured to fit within the recessed portions of the manipulation device. In this manner, the cartridge assembly can be mechanically coupled to the manipulation device. In some embodiments, an adaptor is used to drape all components of the robotic system 102 from the cartridge assembly and potential contaminants.

In some embodiments, the robotic arm can include locking mechanisms to lock and unlock the robotic arm. Locking the robotic arm can prevent further movement of the robotic arm. Unlocking the robotic arm can enable the robotic arm to move as desired. In some embodiments, at 605, the method 600 can include unlocking the robotic arm if the robotic arm is in locked position.

At 606, the method 600 can include moving the robotic arm to position the cartridge assembly at target site. The target site can be a portion of a subject's body on which the medical procedure is to be performed. For example, the target site can be an arm of a patient's body on which the Seldinger technique is to be performed. Moving the robotic arm can include positioning the cartridge assembly at a desired location relative to the target site. For instance, the cartridge assembly can be positioned at an angle with respect to a blood vessel in the target site (e.g., patient's arm). In some embodiments, the angle can be between about 0 degrees and about 90 degrees, between about 10 degrees and about 80 degrees, between about 20 degrees and about 70 degrees, between about 30 degrees and about 60 degrees, between about 40 degrees and about 50 degrees with respect to the blood vessel. In some embodiments, the angle can be between about 20 degrees and about 60 degrees. Additionally or alternatively, the cartridge assembly can be positioned at a specific distance from the blood vessel. In some embodiments, the robotic arm can be moved to position the cartridge assembly such that at least a portion of the cartridge assembly touches the skin of the subject. In some embodiments, moving the robotic arm can include transmitting instructions from an I/O device to the robotic system and/or the robotic arm. For instance, a user can transmit instructions to move the robotic arm via an input device such as joystick, mouse, keyboard, buttons, etc. Once the robotic arm is moved to position the cartridge assembly at the target site, at 607, the robotic arm can be locked to prevent further movement.

At 610, the method 600 can include controlling the manipulation device to perform a vascular access procedure, e.g., the Seldinger technique. In some embodiments, an I/O device (e.g., I/O device(s) 104 in FIG. 1 or user control 404a in FIG. 4) communicably coupled to the robotic system can transmit instructions to the robotic system (e.g., a control unit included in the robotic system) so as to control the movement of the robotic arm. For instance, a control unit (e.g., control unit 405 in FIG. 4) included in the robotic system (e.g., control unit in the base, control unit in the robotic arm, etc.) can process instructions (e.g., instructions from the I/O device) to control the manipulation device. For example, the needle, the catheter, and/or the guidewire can be actuated based on the instructions. Feedback from sensor(s) (e.g., sensor(s) 106 in FIG. 1) and/or imaging device (e.g., imaging device 536 in FIG. 5) can be used for further subsequent control of the manipulation device. For instance, subsequent control of the actuation of the needle, the catheter, and/or the guidewire can be based on feedback from sensor(s) and/or the imaging device.

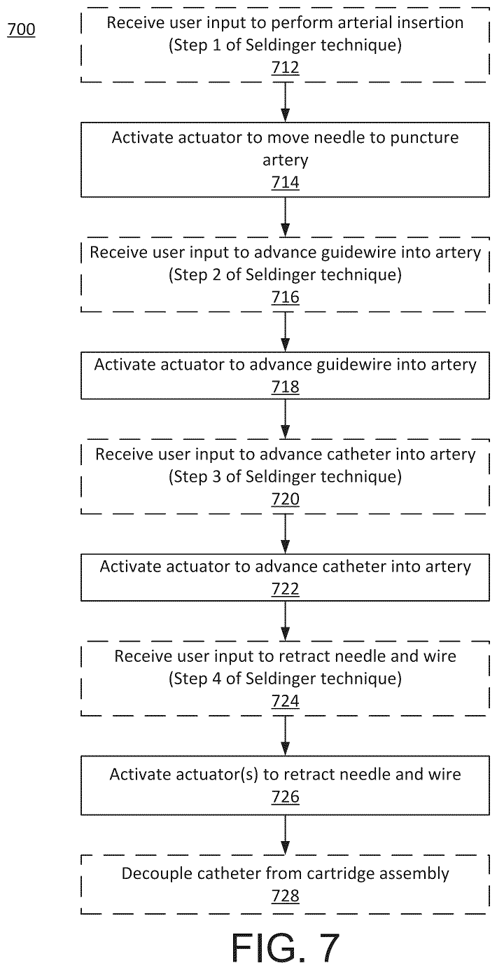

FIG. 7 is a flow diagram illustrating a method 700 of gaining vascular access, for example, by performing the Seldinger technique (e.g., using system 100 in FIG. 1 or any of the other systems and/or components described herein), in accordance with some embodiments. At 712, the method 700 can include receiving user input to access a blood vessel. For instance, the user input can include instructions to perform arterial insertion. In some embodiments, a user can transmit an input to perform the arterial insertion to a robotic system (e.g., robotic system 102 in FIG. 1, robotic system 202 in FIG. 2, etc.) via an I/O device (e.g., I/O device(s) 104 in FIG. 1 or user control 404a in FIG. 4) that is communicably coupled to the robotic system. The robotic system can be controlled based on the input so as to position and/or orient the robotic system to perform the arterial insertion. For example, the base (e.g., base 203 in FIG. 2), the manipulation device (e.g., manipulation device 230 in FIG. 2), and/or the cartridge assembly (e.g., cartridge assembly 240 in FIG. 2) can be positioned and/or oriented based on the user input such that the cartridge assembly is at a specific distance and/or orientation from the desired artery.

At 714, the method 700 can include activating an actuator (e.g., needle actuator 534b in FIG. 5) to move a needle to puncture the artery. In some embodiments, the needle can be included in the cartridge assembly. The needle can be coupled to a needle guide (e.g., a portion of the needle actuator 534b included in the cartridge assembly) that can include a coupling element such as an adaptor. A recessed portion in the manipulation device can be configured to fit the needle guide along with the needle. The cartridge assembly can be attached to the manipulation device via a magnet (e.g., permanent electromagnet) included in a carriage block (e.g., coupling mechanism 538 in FIG. 5) that completes a magnetic circuit with the magnetic portion. The cartridge assembly can also be attached to the manipulation device via a fastener configured to operably couple the cartridge assembly and the manipulation device. In some embodiments, a linear actuator in the manipulation device translates a translation stage coupled to the cartridge assembly including the needle.

In response to the input from the user (e.g., via an I/O device) to perform arterial insertion, a linear actuator (e.g., portion of the needle actuator 534b included in the manipulation device) to actuate the needle can be activated. The linear actuator can move along a linear axis. This in turn can cause the needle guide along with the needle to move along the linear axis. Accordingly, the needle can be moved and positioned so as to puncture the desired artery. In some embodiments, performing the arterial insertion can include aligning a tip of the needle to a catheter tip. As the tip of the needle is advanced into the desired artery, the tip of the needle may remain in the same longitudinal plane. However, the transverse plane of the tip of the needle may change as the needle advances.

In some embodiments, the user can visualize the movement of the needle using visual aid (e.g., ultrasound images) captured by an imaging device (e.g., imaging device 536 in FIG. 5). For example, the absolute position of the tip of the needle can be determined in order to determine the transverse plane that is to be captured through the visual aid. The visual aid can provide the user with information on the movement of the needle. In some embodiments, the user can visualize the movement of the robotic arm, manipulation device, and/or the cartridge assembly as the needle is being actuated using sensor data obtained from sensor(s) (e.g., sensor(s) 106 in FIG. 1). The user can modify the input to perform arterial insertion based on the visual aid and/or the sensor data.

At 716, the method 700 can include receiving user input (e.g., via an I/O device) to advance the guidewire into the artery. Once the artery has been punctured and the needle positioned in the artery, the user can transmit instructions (e.g., similar to step 712) to advance the guidewire into the artery.

At 718, in response to the instructions at 716, the method 700 can include activating an actuator (e.g., guidewire actuator 534c in FIG. 5) to advance the guidewire into the artery. Similar to the needle, the guidewire can be included in the cartridge assembly. The guidewire coupled to a guidewire guide (e.g., a portion of the guidewire actuator 534c included in the cartridge assembly) can be attached to the manipulation device via a fastener or an interface, such as a clutch. Additionally and/or alternatively, a recessed portion in the manipulation device can be configured to fit the guidewire guide along with the guidewire.

In response to the user input (e.g., via an I/O device) to advance the guidewire, a linear actuator (e.g., portion of the guidewire actuator 534c included in the manipulation device and/or the cartridge assembly) to actuate the guidewire can be activated. The linear actuator can move along a linear axis. This in turn can cause the guidewire guide along with the guidewire to move along the linear axis. Accordingly, the guidewire can be advanced to the desired location in the artery. The needle and the catheter can be held stationary as the guidewire is advanced into the artery. In some embodiments, the user can visualize the movement of the guidewire using visual aid captured by the imaging device. The transverse plane that is to be captured via the visual aid can be updated based on the absolute position of a tip of the guidewire. In some embodiments, the user can visualize the movement of the robotic arm, manipulation device, and/or the cartridge assembly as the guidewire is being advanced using sensor data obtained from the sensor(s). The user can modify the input to advance the guidewire based on the visual aid and/or the sensor data.

At 720, the method 700 can include receiving user input (e.g., via an I/O device) to advance the catheter into the artery. In some embodiments, the user can transmit instructions (e.g., similar to step 712) to advance the catheter into the artery.

At 722, in response to the instructions at 720, the method 700 can include activating an actuator (e.g., catheter actuator 534a in FIG. 5) to advance the catheter into the artery. Similar to the needle, the catheter can be included in the cartridge assembly. The catheter coupled to a catheter guide (e.g., a portion of the catheter actuator 534a included in the cartridge assembly) can be attached to the manipulation device via a fastener or an interface, such as a clutch Additionally and/or alternatively, a recessed portion in the manipulation device can be configured to fit the catheter guide along with the catheter.

In response to the user input (e.g., via an I/O device) to advance the catheter, a linear actuator (e.g., portion of the catheter actuator 534a included in the manipulation device and/or the cartridge assembly) to actuate the catheter can be activated. The linear actuator can move along a linear axis. This in turn can cause the catheter guide along with the catheter to move along the linear axis. Accordingly, the catheter can be advanced to the desired location in the artery. In some embodiments, the user can visualize the movement of the catheter using visual aid captured by the imaging device. In some embodiments, the user can visualize the movement of the robotic arm, manipulation device, and/or the cartridge assembly as the catheter is being advanced using sensor data obtained from the sensor(s). The user can modify the input to advance the catheter based on the visual aid and/or the sensor data.

At 724, the method 700 can include receiving user input (e.g., via an I/O device) to retract the needle and the guidewire. At 726, in response to the instructions at 724, the method 700 can include activating the needle actuator and the guidewire actuator to retract the needle and the guidewire. For example, the linear actuators to actuate the needle and the guidewire respectively can be activated such that the linear actuators retract back into the manipulation device along the linear axis. Therefore, the needle guide along with the needle and the guidewire guide along with the guidewire retract into the cartridge assembly along the linear axis (since the needle guide along with the needle and the guidewire guide along with the guidewire are attached to their respective linear actuators). In some embodiments, the user can visualize the movement of the retraction of the needle and the guidewire using visual aid captured by the imaging device. In some embodiments, the user can visualize the movement of the robotic arm, manipulation device, and/or the cartridge assembly as the needle and/or the guidewire is being retracted using sensor data obtained from the sensor(s). The user can modify the input to retract the needle and/or the guidewire based on the visual aid and/or the sensor data.

At 728, the method 700 can include decoupling the catheter from the cartridge assembly. In some embodiments, the catheter can be detached from the catheter guide without releasing the catheter guide from the cartridge assembly. For example, the user can manually decouple the catheter from the catheter guide without decoupling the catheter guide from the cartridge assembly. For example, the catheter may be attached to the catheter guide via a pin assembly and the catheter may be decoupled from the cartridge by removing the at least one pin.

Although in FIG. 7, the sequence of steps to gain access to a blood vessel is described as activating an actuator to advance the needle followed by activating an actuator to advance the guidewire and subsequently activating an actuator to advance the catheter, it should be readily understood that the sequence of steps to gain access to a blood vessel using the system and methods described herein can be performed in any suitable permutations and combinations. For example, in some embodiments, one or more actuators can be activated to advance the needle, the catheter, and the guidewire simultaneously. Once the needle punctures the desired blood vessel (e.g., artery), the guidewire can be advanced distal to the needle to a desired position in the blood vessel. The catheter can then be advanced over the guidewire to the desired position in the blood vessel. In some embodiments, after advancing the guidewire but before advancing the catheter to the desired position in the blood vessel, the needle can be retracted slightly (e.g., moved proximal by a small distance) so that advancing the catheter may be atraumatic to the subject. Alternatively, one or more actuators can be activated to align the needle tip and the distal end of the catheter. The needle and the catheter can be advanced simultaneously to a desired blood vessel. Once the needle punctures the desired blood vessel, the guidewire can be advanced through the puncture to a desired position in the blood vessel. The catheter can then be further advanced to the desired position in the blood vessel. As discussed above, these are a few examples to illustrate various permutations and combinations for accessing a blood vessel using the systems and methods described herein.

In some embodiments, if the size of the blood vessel is large (e.g., central vein), a second catheter can be advanced over the first catheter in order to perform the medical procedure. Put differently, one or more actuators can advance the needle, the guidewire, and the catheter to a desired position in the desired blood vessel. Then, the needle and the guidewire can be retracted from the blood vessel. Another guidewire can be advanced (e.g., manually and/or autonomously) through the catheter already positioned in the desired location. A second catheter that is bigger in size than the already positioned catheter can be advanced through the guidewire. In this manner, the second larger catheter can be positioned through the first catheter in order to perform the medical procedure. In some embodiments, one or more dilators can be used before positioning either the first catheter (e.g., catheter advanced using actuator(s) in the manipulation device and/or cartridge assembly) and/or the second catheter (e.g., catheter that is larger than the first catheter and is advanced through the first catheter) during the medical procedure.

In some embodiments, method 700 as described herein can be performed autonomously and/or semi-autonomously. Accordingly, one or more steps of receiving user input (e.g., 712, 716, 720, 724) can be optional, and systems and devices described herein can be configured to automatically proceed from actuating one component to the next based on confirmation that a first step has been completed. Such confirmation can be determined via sensor data (e.g., via sensor(s) 106) and/or imaging data (e.g., via imaging device 536). In some embodiments, one or more steps may be performed without user input while other steps may be performed with user input.

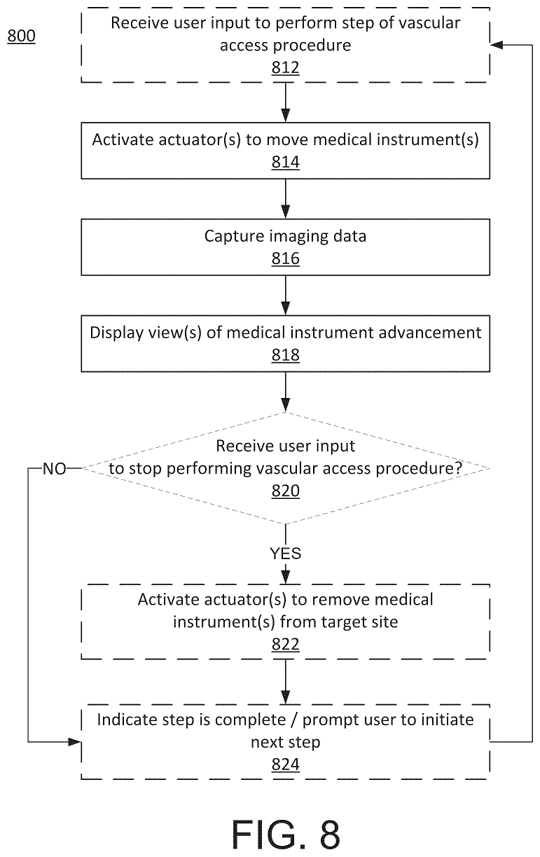

FIG. 8 is a flow diagram illustrating a method 800 of using visual aid and/or sensor data to perform a medical procedure (e.g., using system 100 in FIG. 1), in accordance with some embodiments. At 812, the method 800 can include receiving user input to perform a step of the medical procedure (e.g., vascular access procedure). For example, the user input can include instructions to perform arterial insertion (e.g., step 712 in FIG. 7), advance a guidewire into a blood vessel (e.g., step 716 in FIG. 7), advance a catheter into a blood vessel (e.g., step 720 in FIG. 7), and/or retract a needle and the guidewire from the blood vessel (e.g., step 724 in FIG. 7).

At 814, the method 800 can include activating actuators to move medical instruments based on the user input. For example, activating actuators can include activating actuators within a robotic system (e.g., robotic system 202 in FIG. 2 and/or other robotic systems described herein) to move transport elements (e.g., transport element 414 in FIG. 4) and/or vertical adjustment elements (e.g., vertical adjustment element(s) 409 in FIG. 4) included in the robotic system based on the user input. This in turn can cause a robotic arm (e.g., robotic arm 220 in FIG. 2 and/or other robotic arms described herein), a manipulation device (e.g., manipulation device 230 in FIG. 2 and/or other manipulation devices described herein), and/or a cartridge assembly (e.g., cartridge assembly 240 in FIG. 2 and/or other cartridge assemblies described herein) to be positioned at a desired location from a target site (e.g., subject's body part such as arm). In some embodiments, activating actuators can include activating device actuators (e.g., device actuator(s) 534 in FIG. 5) to advance (e.g., steps 714, 718, and 722 in FIG. 7) and/or retract (e.g., step 726 in FIG. 7) the needle, the catheter, and/or the guidewire into/from a blood vessel based on the user input.

At 816, the method 800 can include capturing visual aid and/or sensor data as the step of the vascular access procedure is being performed. For example, the manipulation device can include an imaging device (e.g., imaging device 536 in FIG. 5) to capture visual aid (e.g., ultrasound images) of the needle, the catheter, and/or the guidewire (collectively device(s) 544 in FIG. 5) as the device(s) are being advanced into and/or retracted from the blood vessel. In some embodiments, the robotic system can be communicably coupled to a sensor (e.g., sensor(s) 106 in FIG. 1) such as a camera to capture images of the robotic system, the manipulation device, and/or the cartridge assembly as the step of the vascular access procedure is being performed.

At 818, the visual aid and/or the sensor data can be displayed on an I/O device (e.g., I/O device(s) 104 in FIG. 1 or user control 404a in FIG. 4). For example, ultrasound images and/or images from cameras can be displayed on a display. At 822, if a user input to stop performing the step of the medical procedure is received (e.g., at step 820), the method 800 can include activating the actuators to remove the medical instruments from the target site. For example, the needle, the catheter, and/or the guidewire can be retracted from the blood vessel by activating the device actuators. Additionally or alternatively, the robotic system, the manipulation device and/or the cartridge assembly can be moved away from the subject by activating the actuators coupled to the transport elements and/or the vertical adjustment elements. In some embodiments, the cartridge assembly can be detached from the manipulation device.

At 824, the method 800 can include indicating to the user that the step of the vascular access procedure is complete. For example, the display can output visual, audio, and/or haptic outputs to represent that the step of the medical procedure is complete. In some embodiments, the display can also prompt the user to initiate the next step thereby repeating the steps of method 800. If the user input to stop performing the step of the medical procedure is not received at step 820, the method 800 can include at 824 indicating to the user that the step of the vascular access procedure is complete and prompting the user to initiate the next step of the vascular access procedure.

### Example Embodiments

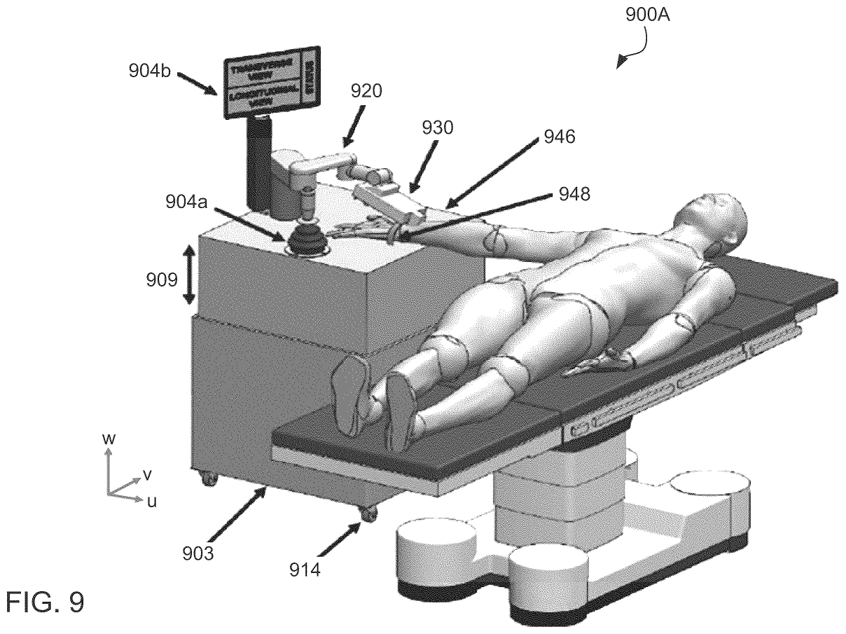

FIG. 9 illustrates a vascular access system 900A (e.g., structurally and/or functionally similar to system 100 in FIG. 1), in accordance with some embodiments. The vascular system can include a robotic system that can include a base 903 (e.g., structurally and/or functionally similar to base 203 in FIG. 2), robotic arm 920 (e.g., structurally and/or functionally similar to robotic arm 220 in FIG. 2), and manipulation device 930 (e.g., structurally and/or functionally similar to manipulation device 230 in FIG. 2).

The base 903 can be a movable base. For example, the base 903 can include transport elements 914 (e.g., structurally and/or functionally similar to transport elements 414 in FIG. 4). In some embodiments, the transport elements 914 can be swivel casters with lockable wheels. The swivel casters can provide the base 903 with three degrees of freedom. For example, the swivel casters can provide translations along the U coordinate axis and the V coordinate axis shown in FIG. 9. The planar and rotational movement can enable the base 903 to be positioned relative to a subject's (e.g., patient on whom the medical procedure is to be performed) arm. In some embodiments, a locking mechanism (e.g., locking mechanism 412 in FIG. 4) for the transport elements 914 can lock a position of the base 903 during the medical procedure. For example, in some embodiments, locks may be engaged automatically during a step of the medical procedure such as upon the base being positioned at an appropriate position relative to the arm of a patient on whom the medical procedure is to be performed.

In some embodiments, the base 903 can include vertical adjustment element(s) 909 (e.g., structurally and/or functionally similar to vertical adjustment element(s) 409 in FIG. 4) which can provide a fourth degree of freedom to the base 903. The vertical adjustment element(s) can raise and/or drop the base vertically, thereby providing the base 903 with a fourth degree of freedom. In some embodiments, the vertical adjustment element(s) 909 can lift a top surface 946 of the base 903 to position the robotic arm 920 at an appropriate height with respect to the patient on whom the medical procedure is to be performed. In some embodiments, the vertical adjustment element(s) 909 can be motorized. For example, the vertical adjustment element(s) 909 can include a ball screw actuator. A fail-safe brake can hold the position of the top surface 946 of the base 903. Linear rails with recirculating balls can constraint the movement of the base 903.

In some embodiments, the top surface 946 of the base 903 can be attached to, integrated with, and/or otherwise coupled to one or more I/O device(s). For example, a display 904b (e.g., structurally and/or functionally similar to display 404b in FIG. 4) can be mechanically coupled to the top surface 946 of the base via a display support. The display 904b can provide visual aid and/or visual feedback including longitudinal and traverse ultrasound views of a blood vessel of the subject. In some embodiments, the display 904b can provide state and status information on the task being performed during the medical procedure. Some examples of state information can include the state of the needle actuator, guidewire actuator, and/or catheter actuator during each step of the medical procedure as seen in Table 1.

TABLE 1

| Medical | | Guidewire | Needle | Catheter |
|---|---|---|---|---|
| procedure | | Actuator | Actuator | Actuator |
| Step | Description | (motor) | (motor) | (motor) |
| 1 | Insertion into blood | Dynamic | Dynamic | Dynamic |
| | vessel | | | |
| 2 | Advance guidewire | Dynamic | Static | Static |
| | into the blood vessel | | | |
| 3 | Advance catheter into | Static | Static | Dynamic |
| | the blood vessel | | | |
| 4 | Retract needle and | Dynamic | Dynamic | Static |
| | guidewire | | | |

In some embodiments, a user control 904a (e.g., structurally and/or functionally similar to user control 404a in FIG. 4) can be attached to and/or integrated with the top surface 946 of the base 903. For example, a joystick 904a as shown in FIG. 9 can be used to control the robotic system. For instance, the joystick 904a may control the three degrees of freedom (e.g., translation and rotation) and/or four degrees of freedom (e.g., translation, rotation, and/or vertical drop or raise) of the base 903. The joystick 904a can be any suitable type of joystick such as digital joysticks, paddle joysticks, analog joysticks, Pc analog joysticks, and/or the like.

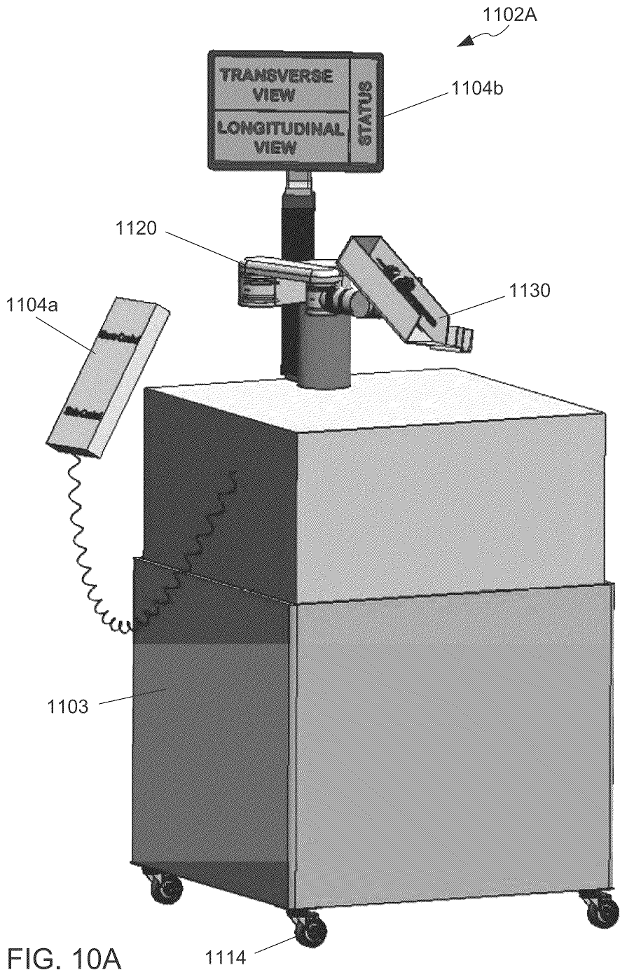

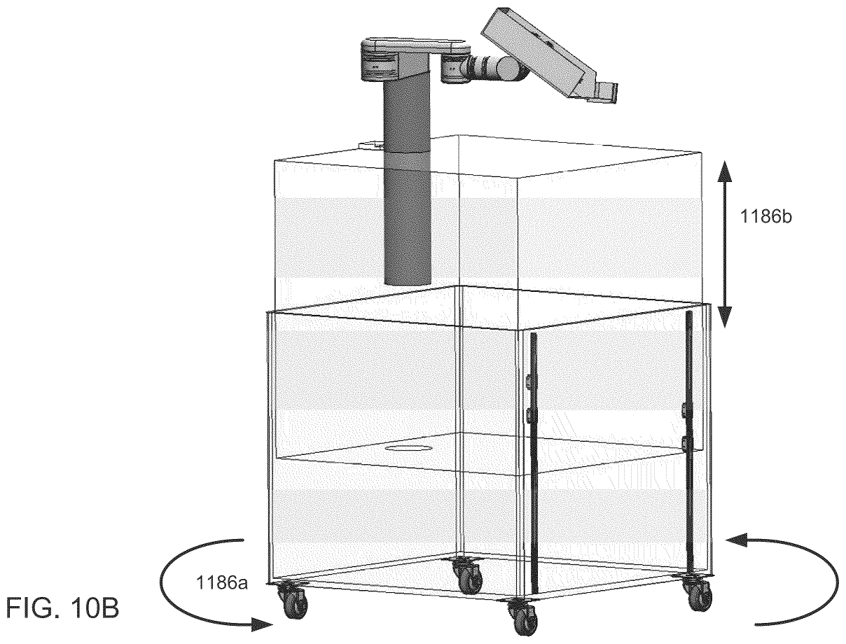

Additionally or alternatively, a handheld pendant (e.g., handheld pendant 1104a in FIG. 10A further described below) can be used to control the robotic system. In addition to controlling the three and/or four degrees of freedom of the base 903, the handheld pendant can additionally control the needle actuator, guidewire actuator, and/or catheter actuator prior to, during, and/or after each step of the Seldinger technique based on the states shown in Table 1. For example, the handheld pendant can change the state of the needle actuator and the catheter actuator from dynamic to static after arterial insertion but before advancing the guidewire into the artery.

In some embodiments, the top surface 946 of the base 903 can be attached to, integrated with, and/or otherwise coupled to the robotic arm 920. In some embodiments, the top surface 946 of the base 903 can include an arm restraint 948 to constrict the arm of a subject during the medical procedure. The arm restraint 948 can be an elastic band that can restraint an arm of any size.

The sensor 906 can be configured to be controlled remotely using one or more I/O devices. For instance, the sensor 906 can be controlled by an I/O device that is communicably coupled to the robotic system and/or base 903. A user controlling the sensor 906 can be at a location remote to the system 900B. For example, the sensor 906 can be controlled via an integrated computing device such as computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc. that is communicably coupled to the robotic system and/or base 903. In some embodiments, the user control 904a can enable a user in proximity and/or within the vicinity of the system 900B to control the sensor 906.

The sensor 906 can capture a view of the environment for the remote user. Controlling the pan/tilt mechanism can allow the sensor 906 to capture image data from various angles. A user can change the view of the environment as desired by controlling the pan/tilt mechanism. The image data can be used as feedback by the user to control the robotic system in order to perform the medical procedure. In some embodiments, instead of a user controlling the pan/tilt mechanism via an I/O device such as user control 904a, the entire system (e.g., system 900A in FIG. 9A and/or system 900B in FIG. 9B) can be autonomous. For instance, a control unit within the system can control the user control 904a and/or pan/tilt mechanism 966 by taking into account the feedback from image data and/or visual aid (e.g., ultrasound data) as the robotic system performs the medical procedure. Put differently, the system 900A and/or 900B can perform the medical procedure in an autonomous manner.

In some embodiments, the system may include an additional image sensor such as stereo camera (not shown in FIG. 9) to enable precise motion of the robotic arm 920 and/or manipulation device 930. The additional image sensor can allow precise targeting and/or access of the vascular portion. For instance, the feedback from the additional image sensor can control the manipulation device 930 and/or the robotic arm 920 in a more precise manner. In some embodiments, proximity sensor(s) may be attached to, coupled to, and/or otherwise mounted on the base 903 to enable previse motion and targeting.

In some embodiments, when the manipulation device 930 contact the skin of the subject, the feedback can be switched from sensor 906 feedback to visual aid from an imaging device such as ultrasound array. For example, a user remote to the system can switch the feedback to ultrasound using an I/O device. As discussed above, in some embodiments, the robotic arm 920 can be motorized. The motorized arm can be controlled (e.g., via an I/O device) to achieve a desired view of a target vascular portion of the subject in the ultrasound array.

In some embodiments, the robotic arm 920 can include sensors to measure force and/or torque in order to perform the medical procedure in a safe manner. For instance, a needle penetration force that is greater than a threshold value can cause damage to the skin, blood vessel, and/or neighboring tissues. Accordingly, measuring the force and/or torque during the medical procedure can ensure the needle penetration force is below the threshold value. For example, for a 25G needle the maximum penetration force that can be applied by the needle to puncture a forearm vein is 2.5 N. Similarly, maximum penetration force can be determined for 18G needle, 22G needle, etc. If the penetration force measured by the sensors exceed the identified maximum penetration value, in some embodiments, the system 900A and/or 900B can be automatically shut down. For example, in response to the penetration force exceeding the penetration value a control unit (e.g., structurally and/or functionally similar to control unit 405 in FIG. 4) can automatically shut down the system. In some embodiment, the measured force can also be an indicator of whether or not the needle may have penetrated into a tissue of the subject. For example, when the needle penetrates the tissue, the penetration force can drop. In such scenarios, a user can be notified via a display (e.g., structurally and/or functionally similar to display 404b in FIG. 4) that the needle has penetrated the tissue. In some embodiments, the robotic arm 920 can include collision sensing skin sensors to identify whether the manipulation device 930 is in contact with the skin of the subject prior to performing the medical procedure.

FIG. 10A illustrates a robotic system 1102A (e.g., structurally and/or functionally similar to robotic system 202 in FIG. 2), according to some embodiments. The robotic system 1102A includes a base 1103. A user control 1104a can be attached to, coupled to, and/or integrated with the base 1103. In some embodiments, the user control 1104a can be a handheld pendant. The handheld pendant 1104a can be coupled to the base 1103 via a spiral cord. As discussed above, the handheld pendant 1104a can control the linear motion, rotational motion, and/or vertical movement of the base 1103. Additionally, the handheld pendant 1104a can change the state of the system itself or otherwise one or more components of the system (e.g., needle actuator, catheter actuator, guidewire actuator, etc.) from one step of the medical procedure (e.g., the Seldinger technique) to the next step of the medical procedure. Some non-limiting examples of the state of some components for each step of the Seldinger technique is shown in table 1. For example, the handheld pendant 1104a can be used to change the state of the guidewire actuator from dynamic to static and the state of the catheter actuator from static to dynamic after advancing the guidewire into the artery of a subject but before advancing the catheter into the artery of the subject.

In some embodiments, a display 1104b can be attached to, coupled to, and/or integrated with the base 1103. The display 1104b can display visual aid (e.g., transverse view and longitudinal view of ultrasound images), image data (e.g., images captured from a sensor such as camera), status, and state of the task being performed. In some embodiments, a robotic arm 1120 can be attached to, coupled to, and/or integrated with the base 1103. In some embodiments, the robotic arm 1120 can be motorized. In some embodiments, an end segment of the robotic arm 1120 can be coupled to a manipulation device 1130. The manipulation device can be attached to a cartridge assembly.

In some embodiments, the base 1103 can include transport elements 1114 such as swivel wheels. The transport elements 1114 can provide three degrees of freedom to the base 1103. FIG. 10B illustrates rotational motion and vertical movement for a base 1103, according to some embodiments. The transport elements 1114 can allow the base 1103 to rotate along 1186a. Additionally, the transport elements 1114 can allow the base 1103 to move linearly along two perpendicular axes, thereby providing three degrees of freedom. Additionally, the base 1103 can include vertical elements to raise and/or drop the base 1103 along 1186b. This can provide a fourth degree of freedom to the base.

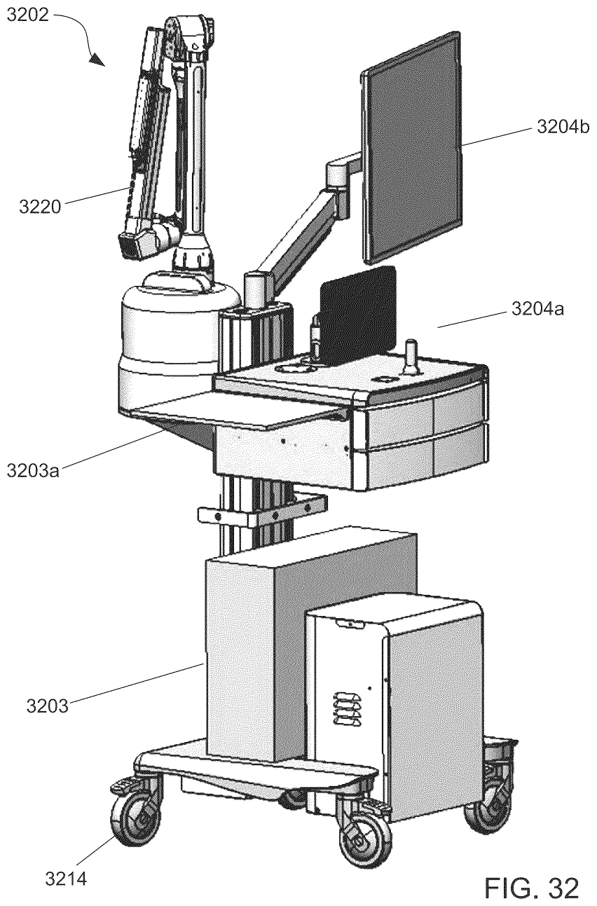

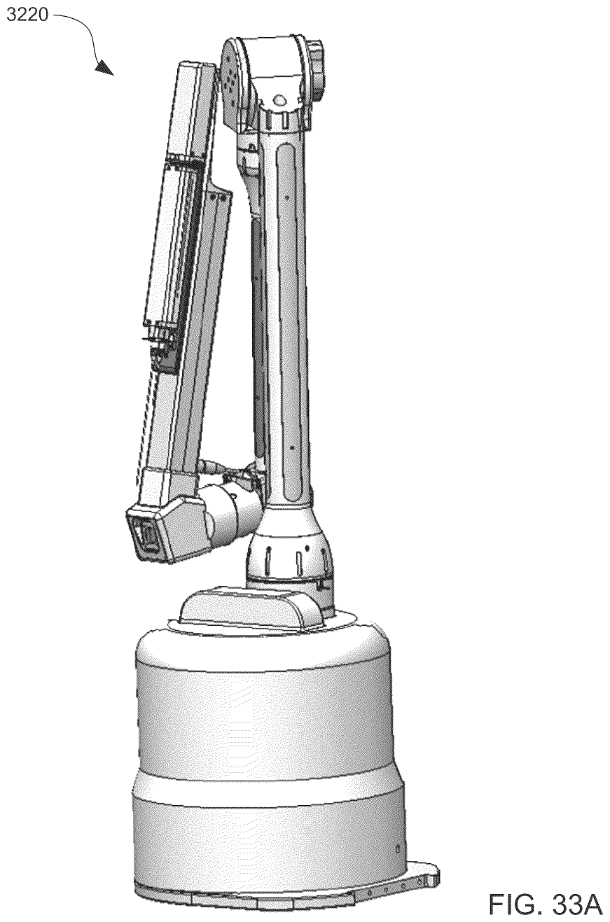

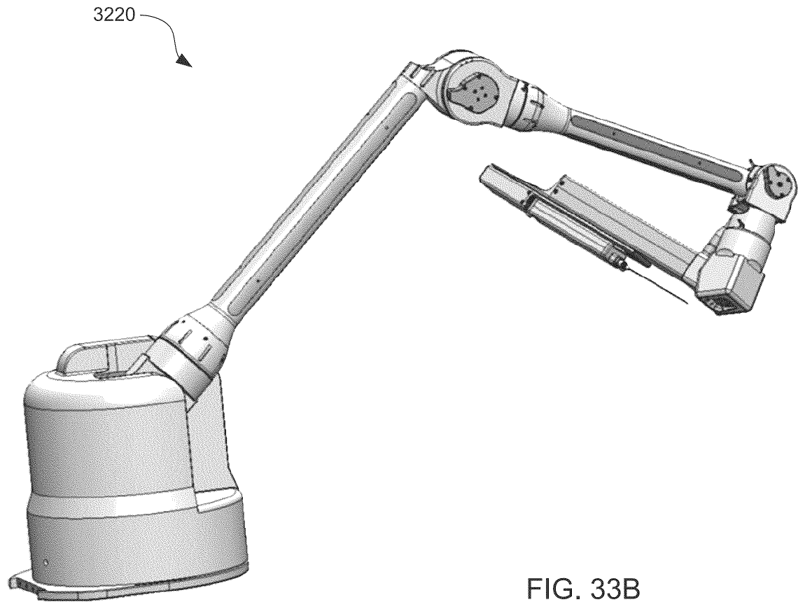

FIGS. 32-33B illustrate another example of a robotic system 3202, according to some embodiments. The robotic system 3202 can be structurally and/or functionally similar to other robotic systems described herein, including, for example, robotic system 202, robotic system 1102, etc.

FIG. 32 illustrates a perspective view of the robotic system 3202, in accordance with some embodiments. As shown, the robotic system 3202 can include a base 3203 (e.g., functionally and/or structurally similar to the base 1103 of FIG. 10A). The base 3203 can include transport elements 3214 (e.g., structurally and/or functionally similar to the transport elements 1114 off FIG. 10A). The transport elements 3214 vary from the transport elements 1114 by only providing two degrees of freedom (e.g., allow the robotic system 3202 to move around a surface). The vertical height of the base 3203 is fixed to a predetermined height.

The base 3203 may be coupled to a user control interface 3204a. The user control interface 3204a includes a touchpad and a joystick to control the operations of the robotic system 3202. In some embodiments, the base 3203 is coupled to a robotic arm 3220 (e.g., structurally and/or functionally similar to the robotic arm 1120 of FIG. 10A). The user control interface 3204a may be utilized to control the functionality of the robotic arm 3220. For example, the touchpad may be utilized to alter or activate operations of the robotic arm 3220, while the joystick may be utilized to control the position of the robotic arm 3220. The base 3203 attached to, coupled to, and/or integrated with a display 3204b which may display information display visual aid (e.g., transverse view and longitudinal view of ultrasound images), image data (e.g., images captured from a sensor such as camera), status, and state of the task being performed.

The base 3203 may include an arm support stand 3203a. In some embodiments, the arm support stand 3203a couples to the base 3203 via a rail. In some embodiments, the base 3203 includes multiple rails for attaching the arm support stand 3203a. In some embodiments, the arm support stand 3203a may be removed, folded, or otherwise repositioned relative to the base 3203.

FIG. 33A illustrates a robotic arm 3220 of the robotic system 3202 of FIG. 32 in a first configuration for storage. In the first configuration, the robotic arm 3220 is positioned so that the robotic arm 3220 folds into a compact shape. The first configuration may reduce the likelihood that the robotic arm 3220 is damaged when not in use. In some embodiments, the robotic arm 3220 may include a locking mechanism that locks the robotic arm 3220 in the first configuration. FIG. 33B illustrates the robotic arm in a second configuration for vascular access. In the second configuration, the robotic arm is deployed and prepared for performing a vascular access procedure.

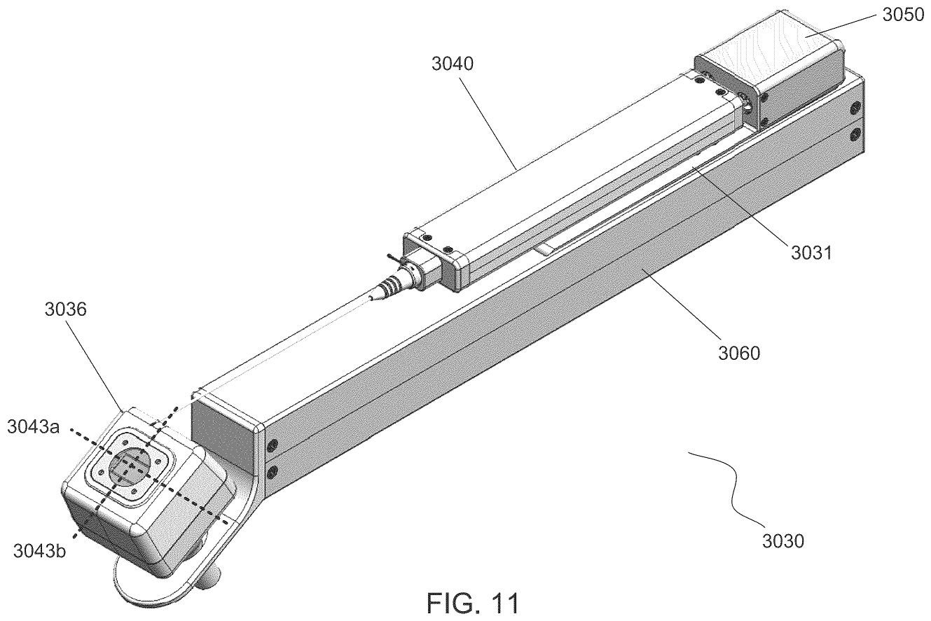

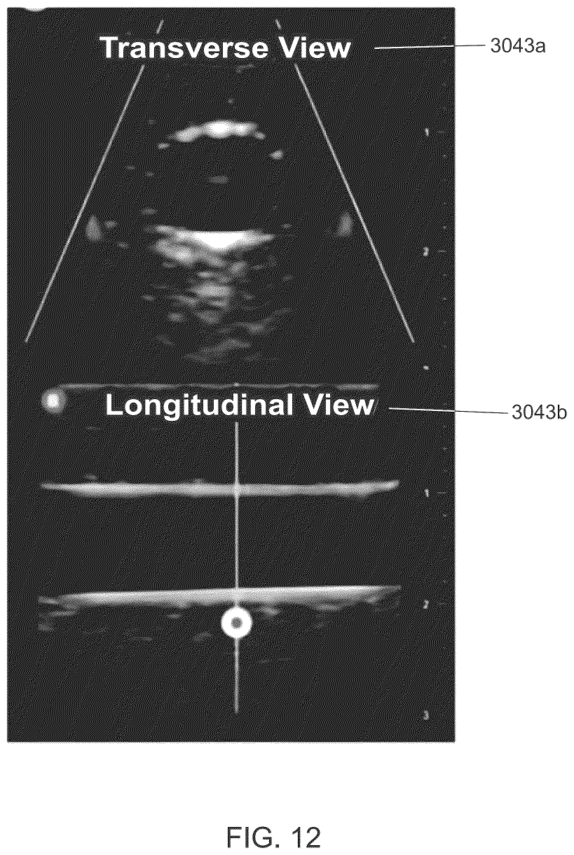

FIG. 11 illustrates a manipulation device 3030 (e.g., structurally and/or functionally similar to other manipulation devices described herein, including the manipulation device 230 in FIG. 2), in accordance with some embodiments. The manipulation device 3030 includes a body 3060 and a cartridge motor assembly 3050. The body 3060 and the cartridge motor assembly 3050 provide components that drive the needle, catheter, and/or guidewire. The manipulation device 3030 is coupled to a cartridge assembly 3040 (e.g., structurally and/or functionally similar to other cartridge assemblies described herein, including the cartridge assembly 540 in FIG. 5). The cartridge assembly 3040 can be attached to the manipulation device 3030 by engaging the cartridge assembly 3040 with the cartridge motor assembly 3050 such that the actuators within the cartridge assembly 3040 are operably coupled to the motors in the cartridge motor assembly 3050. In some embodiments, one or more mechanical engagement features between the cartridge assembly 3040 and the manipulation device 3030 can mechanically couple the cartridge assembly 3040 to the manipulation device 3030. For example, the housing of the cartridge assembly 3040 can include features such as tabs that fit within slots in the housing of the manipulation device 3030. The cartridge assembly 3040 may be coupled to and/or supported by a translation plate 3031 of the manipulation device 3030. The translation plate 3031 is configured to slide along the body 3060 and translates both the cartridge assembly 3040 and the cartridge motor assembly 3050 during operation.

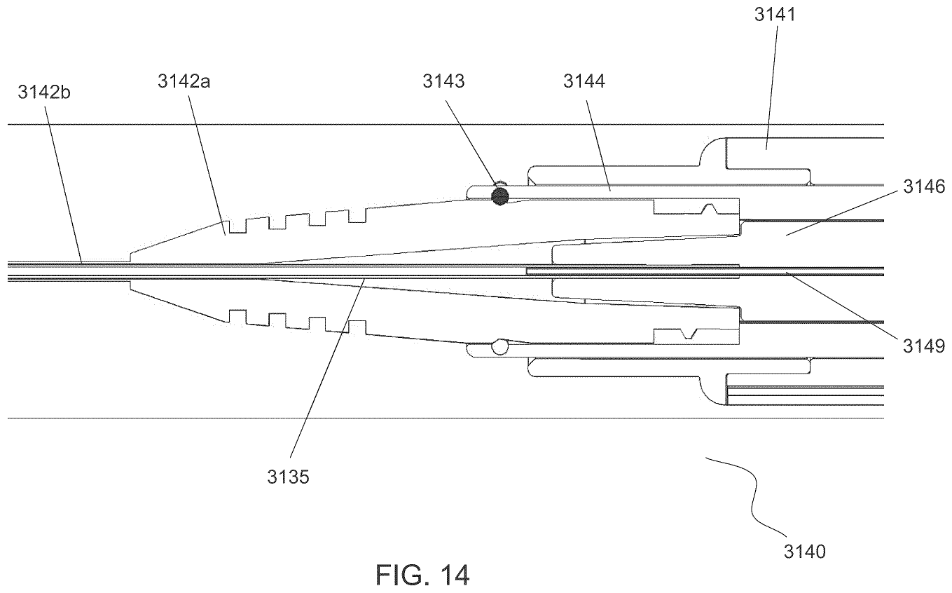

The manipulation device 3030 can include an imaging device 3036 (e.g., structurally and/or functionally similar to imaging device 536 in FIG. 5) that can provide a user with a visual aid of a vascular portion (e.g., blood vessel) as the medical procedure is being performed. In some embodiments, the imaging device 3036 can be an ultrasound imaging device that captures a visual representation of a blood vessel. In some embodiments, the imaging device 3036 can be an ultrasound array located on the manipulation device 1430. The ultrasound array can provide two-dimensional ultrasound images along a longitudinal plane 1443b and a transverse plane 1443a. FIG. 12 illustrates a traverse view 1543a and a longitudinal view 1543b of a blood vessel as captured by an ultrasound imaging device (e.g., imaging device 1436 in FIG. 14).

As seen in FIG. 12, the transverse view 3043a of the blood vessel shows a radial cross section of the blood vessel while the longitudinal view 3043b of the blood vessel shows an axial cross section of the blood vessel. As discussed above, the ultrasound array can provide a visual aid of the movement of the needle, catheter, and/or guidewire into the blood vessel. Subsequent movement of the manipulation device can be controlled based on the feedback from the visual aid.

As the needle advances into the field of view of the ultrasound array, the tip of the needle may remain in a central longitudinal plane (e.g., longitudinal plane 3043b in FIG. 11) of the ultrasound array and the manipulation device because the ultrasound array and the manipulation device and/or the robotic arm are physically connected to each other. However, as the tip of the needle advances into the blood vessel, the transverse plane of the needle may not remain in the transverse plane (e.g., transverse plane 3043a in FIG. 11) of the ultrasound array.

In some embodiments, a position encoder on the needle actuator (e.g., needle actuator 534b in FIG. 5) and a limit switch can be used to determine the absolute position of the needle tip. The absolute position of the needle tip can be used to determine which transverse plane to display as a visual aid to the user.

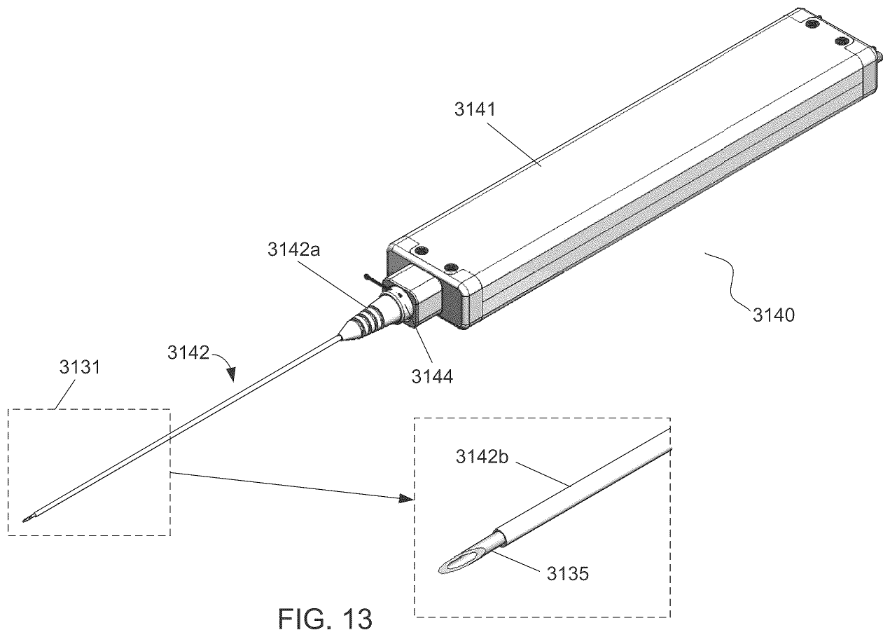

FIGS. 13-21B illustrate different views and portions of a cartridge assembly 3140 (e.g., structurally and/or functionally similar to other cartridge assemblies described herein, including the cartridge assembly 240 in FIG. 2 and/or the cartridge assembly 3040 in FIG. 11), in accordance with some embodiments. The cartridge assembly 3140 may be disposable, or certain portions of the cartridge assembly can be reusable after appropriate disinfection and sanitation procedures. The cartridge assembly 3140 can be sterile such that it can be exposed to the patient and potential contaminants, while allowing for the other components of the robotic system, such as robotic system 202, to be shielded behind a drape. As the cartridge assembly 3140 is removable from the robotic system, the cartridge assembly 3140 may be replaced with a new cartridge assembly for a subsequent vascular access procedure.

The cartridge assembly 3140 includes a cartridge body 3141, a catheter 3142 including a catheter hub 3142a and a catheter shaft 3142b, a needle 3135, and a guidewire 3149. The cartridge body 3141 houses one or more linear actuators 3134a, 3134b, such as the linear actuators corresponding to the catheter and the guidewire (e.g., structurally and/or functionally similar to actuator(s) 534a, 534c). The cartridge body 3141 also protects the internal components of the cartridge assembly 3140 from contaminants that may affect the operation of the cartridge assembly 3140. The catheter 3142 can be selectively driven, e.g., via a linear actuator 3134a (e.g., structurally and/or functionally similar to the catheter actuator 534a in FIG. 5). The guidewire 3149 can be selectively driven, e.g., via a linear actuator 3134b (e.g., structurally and/or functionally similar to the guidewire actuator 534c in FIG. 5). The catheter 3142 and the guidewire 3149 can be coupled to the linear actuators 3134a, 3134b, respectively, via one or more guide elements. For example, the catheter 3142 can be coupled to the linear actuator 3134a via guide elements 3144, 3145a. The catheter guide element 3144 can be coupled to the catheter hub 3142a. In some embodiments, the coupling can be releasable, e.g., via removal of a pin 3143, as described in further detail below. The catheter guide element 3144 can be coupled to a catheter guide element 3145a, which in turn is coupled to a follower 3148a (e.g., a follower nut or screw nut) of the linear actuator 3134a. The follower 3148a can advance along a longitudinal axis of a linear shaft 3139a of the linear actuator 3134a, e.g., when the linear shaft is rotated. The guidewire 3149 can be coupled to the linear actuator 3134b via guide element 3145b. The guide element 3145b can be coupled to a follower 3148b (e.g., a follower nut or screw nut) of the linear actuator 3134b. The follower 3148b can advance along a longitudinal axis of a linear shaft 3139b of the linear actuator 3134b, e.g., when the linear shaft is rotated.

FIG. 13 illustrates a detailed view of a distal end portion 3131 of the cartridge assembly 3140. As shown in the detailed view, the needle 3135 is disposed within the catheter shaft 3142b. While the distal end of the needle 3135 is depicted slightly extended from the distal end of the catheter shaft 3142b, it can be appreciated that the distal end of the needle 3135 can be disposed more distal of or aligned with the distal end of the catheter shaft 3142b. In use, the distal end of the needle 3135 can be advanced to puncture through tissue and into a vessel, allowing the catheter 3142 and the guidewire 3149 to be advanced into the vessel.

FIG. 14 illustrates a section view of an area around a proximal end of the catheter 3142 of the cartridge assembly 3140, in accordance with some embodiments. As noted previously, the catheter includes a catheter hub 3142a and a catheter shaft 3142b extending from the catheter hub 3142a. A needle 3135 can be disposed within the catheter shaft. In some embodiments, the needle 3135 can be coupled to a needle guide 3146 disposed within the catheter guide 3144. The needle guide 3146 can be structurally and/or functionally similar to the needle guide 544b described with reference to FIG. 5. A guidewire 3149 can be disposed within the needle 3135 and the needle guide 3146.

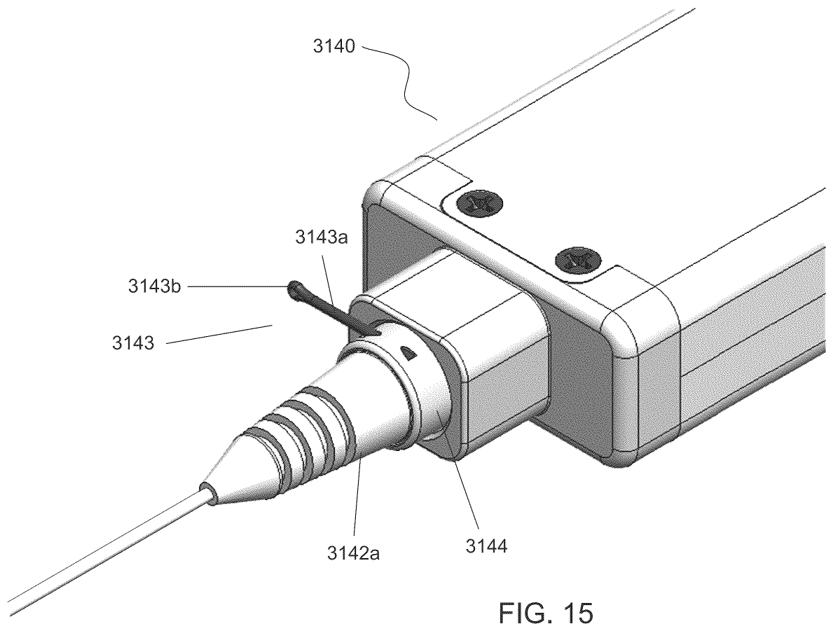

In some embodiments, the catheter hub 3142a is coupled to the catheter guide 3144 via a removable pin 3143. Alternatively or additionally, the catheter may also be removable coupled to the catheter guide 3144 via other mechanism, e.g., via friction fit, screw engagement, peelable or breakable adhesives, etc. FIG. 15 illustrates the catheter hub 3142a coupled to the catheter guide 3144 via the pin 3143. A pin shaft 3143a of the pin 3143 presses against the catheter hub 3142a to hold it in place relative to the catheter guide 3144. The pin 3143 may be removed from the catheter guide 3144, e.g., by pulling on a pin head 3143b. When the pin 3143 is removed, the catheter hub 3142a can be released from the catheter guide 3144. This can allow the catheter 3142 to be separate from the remaining components of the cartridge assembly 3140. In use, the cartridge assembly 3140, coupled to a manipulation device, can be used to place a catheter (e.g., catheter 3142) into a vessel of a patient, and then the catheter can be decoupled from the cartridge assembly 3140, leaving it in place in the vessel for further medical use.

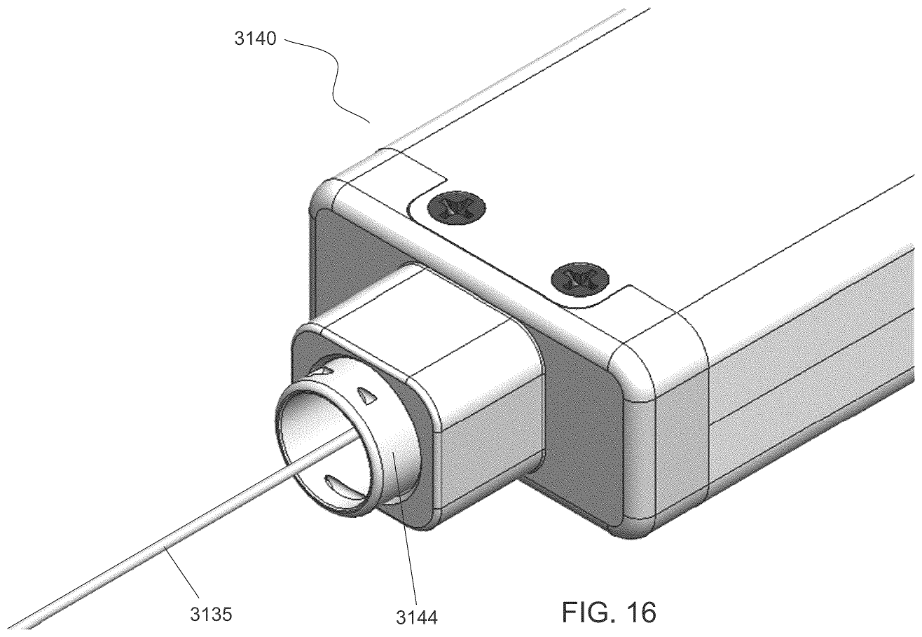

FIG. 16 illustrates the cartridge assembly 3140 with the catheter 3142 removed, in accordance with some embodiments. For example, FIG. 16 may illustrate a catheter assembly 3140 after a pin, such as pin 3143, has been removed to decouple and remove the catheter hub 3142a. The catheter 3142 may be removed from the cartridge assembly 3140 after the pin is removed, e.g., by sliding the catheter hub 3142a out of the catheter guide element 3144. In some embodiments, the catheter is removed manually by a medical professional. In some embodiments, the catheter hub 3142a can be separated from the cartridge assembly 3140 via the robotic system. For example, the pin 3143 can be removed, e.g., using a robotic manipulator, and the cartridge assembly 3140 can be retracted away from the catheter 3142, thereby leaving the catheter 3142 in place while the remaining portions of the cartridge assembly 3140 are move away from a surgical site. The site can then be prepared for a medical procedure.

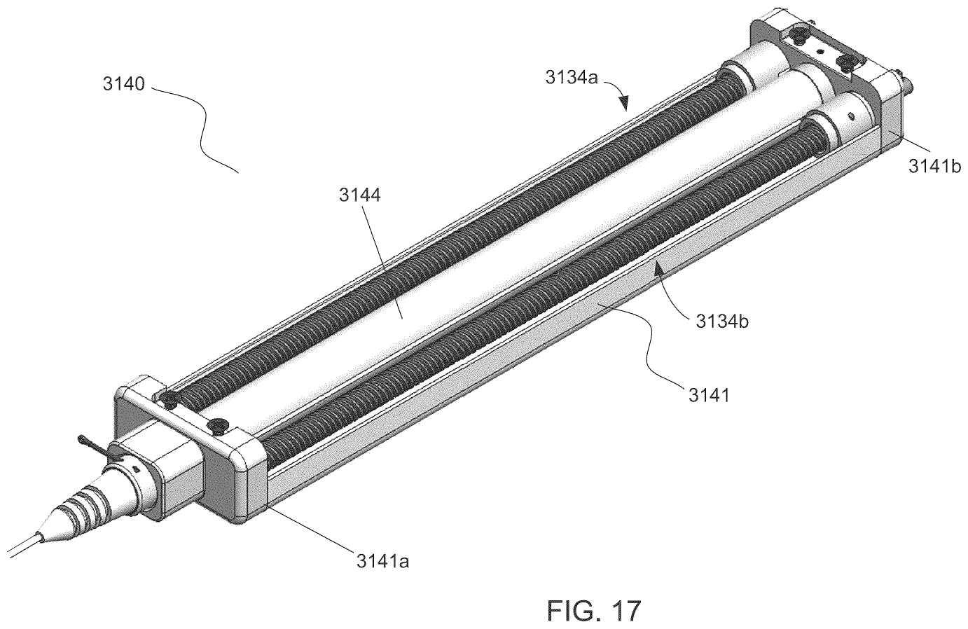

FIG. 17 illustrates the cartridge assembly 3140 with a portion of the housing of the cartridge body 3141 removed to show certain internal components of the cartridge assembly 3140. As described above, the cartridge assembly 3140 includes one or more actuator(s) 3134a, 3134b (e.g., structurally and/or functionally similar to device actuator(s) 534 of FIG. 5). The actuator(s) 3134a, 3134b can each include a linear shaft 3139a, 3139b (e.g., linear screw shaft) and a follower 3148a, 3148b (e.g., follower screw or screw nut). In some embodiments, the linear shafts 3139a, 3139b can extend between a first housing end 3141a and a second housing end 3141b. Alternatively, the linear shaft 3139a, 3139b can extend a portion of the cartridge body 3141. The first housing end 3141a includes an aperture or opening through which the catheter, needle, and guidewire can extend. The second housing end 3141b includes one or more apertures or openings through which the actuators 3134a, 3134b may couple with a motor (e.g., a rotational motor) or other drive mechanism. In some embodiments, each of the actuators 3134a, 3134b can be in a direct drive configuration (e.g., does not include a geared power transfer) with a motor or drive mechanism. In some embodiments, one or more of the actuators 3134a, 3134b includes or is coupled to a transmission to transmit motion from a drive mechanism to linear shafts 3139a, 3139b of the actuators.

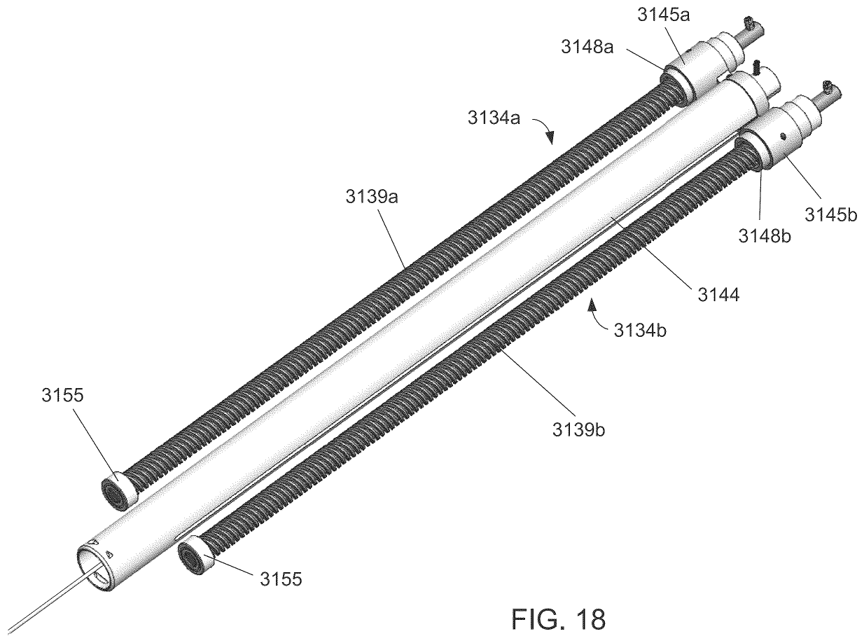

FIG. 18 provides a more detailed view of the internal components of the cartridge assembly 3140, in accordance with some embodiments. The actuators 3134a, 3134b are configured to control a position of the catheter 3142 and the guidewire 3149, e.g., by translating them relative to the cartridge body 3141. In some embodiments, the catheter actuator 3134a and the guidewire actuator 3134b can be ball screw assemblies that operate via the corresponding linear shafts or screws 3139a and 3139b, which rotate to translate the followers 3148a and 3148b (and guides 3145a and 3145b coupled thereto) along the linear shafts 3139a and 3139b. In some embodiments, the catheter actuator 3134a and the guidewire actuator 3134b may be independently controlled, e.g., to allow for independent control of the movement of the catheter and the guidewire (e.g., independent insertion, advancement, and/or retraction).

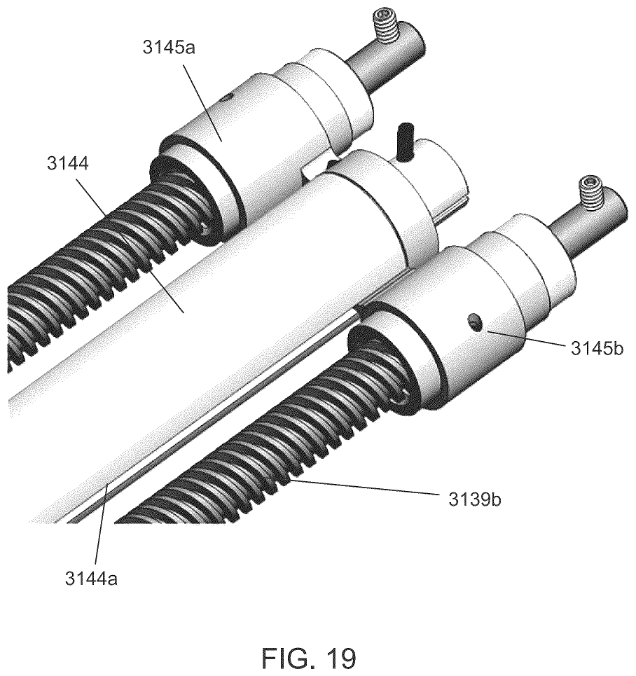

FIG. 19 provides a close-up view of a catheter guide element 3145a coupled to the catheter guide element 3144. The catheter guide element 3145a can include a collar or ring that is disposed around the catheter guide element 3144. In some embodiments, the catheter guide element 3145a may be coupled to the catheter guide element 3144 via a press-fit, adhesive (e.g., glue, epoxy, etc.), screw mechanism, or another fastener (e.g., clip, pin, set-screw, etc.). FIG. 19B also illustrates a slot 3144a disposed within the catheter guide element 3144, which allows for guidewire guide element 3145b to extend into the catheter guide element 3144 and couple to the guidewire disposed therein. The slot 3144a can be designed to allow the guidewire to be advanced via the linear actuator 3134b relative to the catheter guide element 3144 and the catheter 3142. In some embodiments, the slot 3144a can have a length that is limits a distance that the guidewire can be advanced distally relative to the catheter guide element 3144. In some embodiments, the slot 3144a along with the catheter guide element 3144 can provide additional support to the guidewire and/or guidewire guide element, e.g., to prevent bending, kinking, folding, and/or other undesirable motion of the guidewire and/or guidewire guide element.

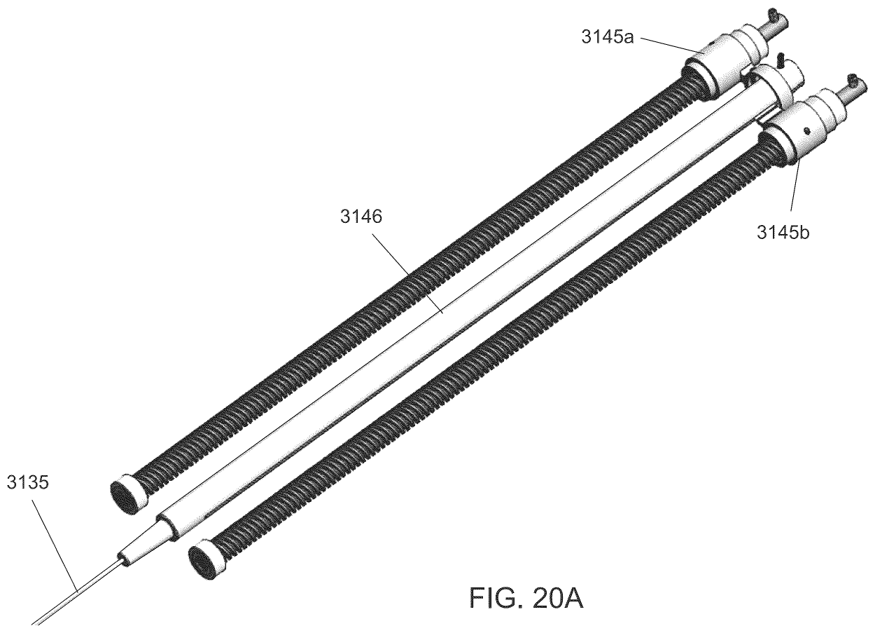

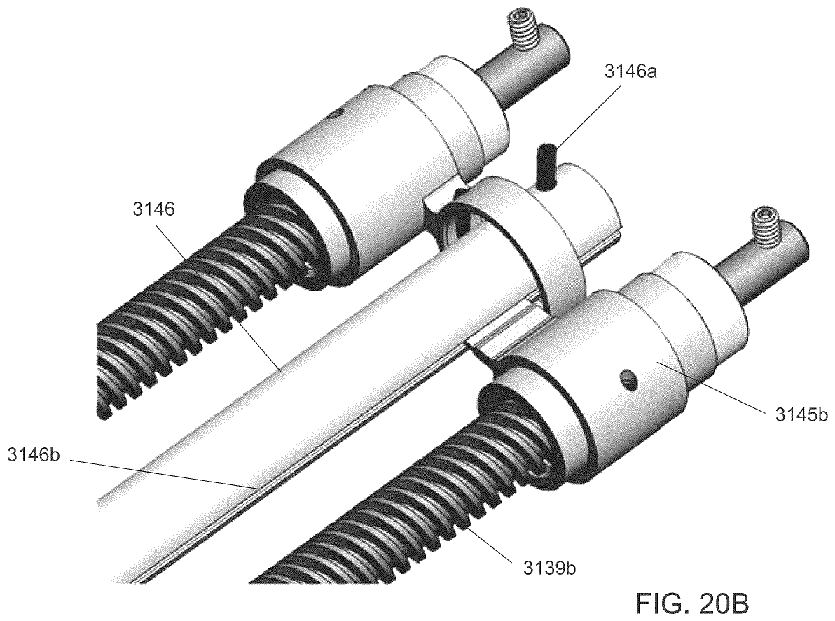

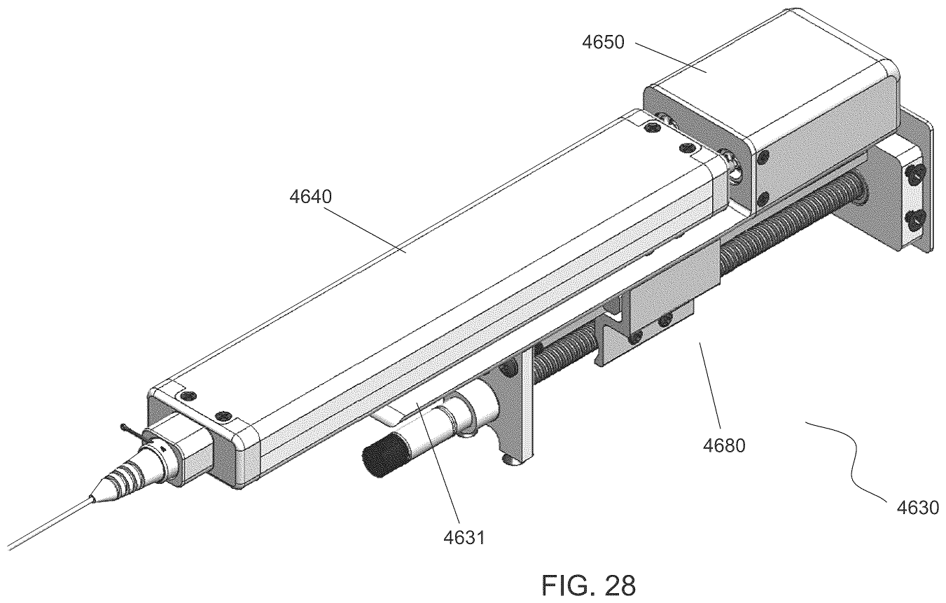

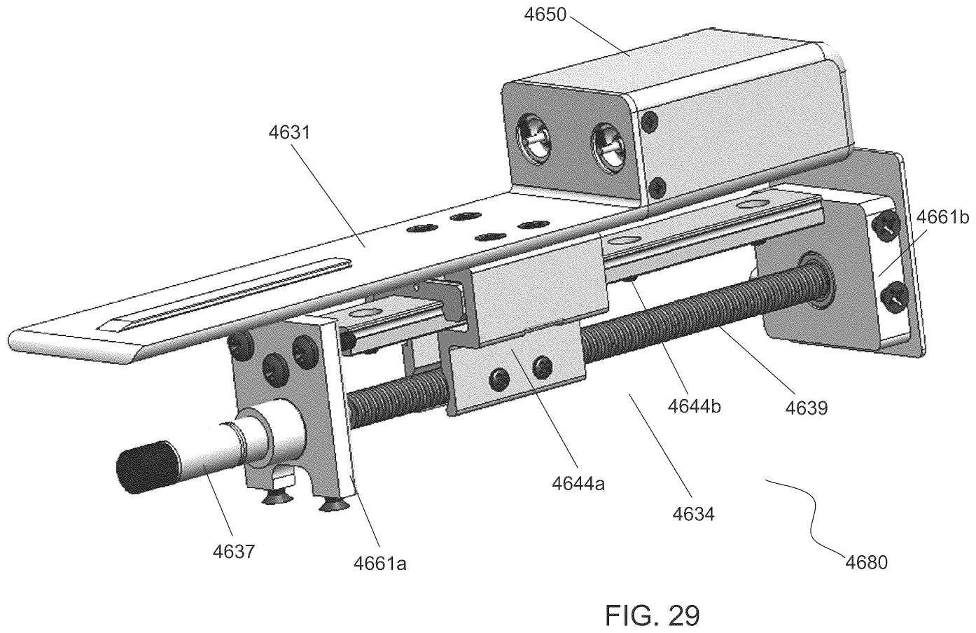

FIG. 20A illustrates the cartridge assembly 3140 with the outer housing removed and with the catheter guide element 3144 removed, such that a needle guide element 3146 (structurally and/or functionally similar to needle guide 544b) is viewable, in accordance with some embodiments. The needle guide 3146 is coupled to the needle 3135 and can hold the needle 3135 in a fixed position relative to the cartridge body 3141. The needle guide 3146 can be independently formed form the catheter guide elements 3144, 3145a and the guidewire guide element 3145b. The needle guide element 3146, and thus the needle 3135, translate together with the cartridge body 3141. In some embodiments, a separate actuator (not depicted in FIGS. 20A and 20B) can be used to translate the cartridge body 3141. For example, an actuator disposed in a manipulation device (e.g., such as that depicted in FIGS. 28 and 29) can be configured to translate the cartridge body 3141. Such an actuator can also include a ball screw assembly, or similar translating assembly, for translating the cartridge body 3141. In use, translation of the cartridge body 3141 can cause translation of the needle guide element 3146 and correspondingly the needle 3135, the catheter guide elements 3144, 3145a and correspondingly the catheter 3142, and the guidewire guide element 3145b and correspondingly the guidewire 3149.

FIG. 20B provides a close-up view of a proximal end of the needle guide element 3146 including a coupling pin 3146a. The coupling pin 3146a couples the needle guide 3146 to the housing of the cartridge body 3141 (e.g., the second housing end 3141b). The coupling pin 3146a can be a fastener or other type of mechanical and/or adhesive structure. The needle guide element 3146 also includes a needle guide slot 3146b which allows for the guidewire guide element 3145b to extend into the needle guide element 3146 and couple to the guidewire disposed therein.

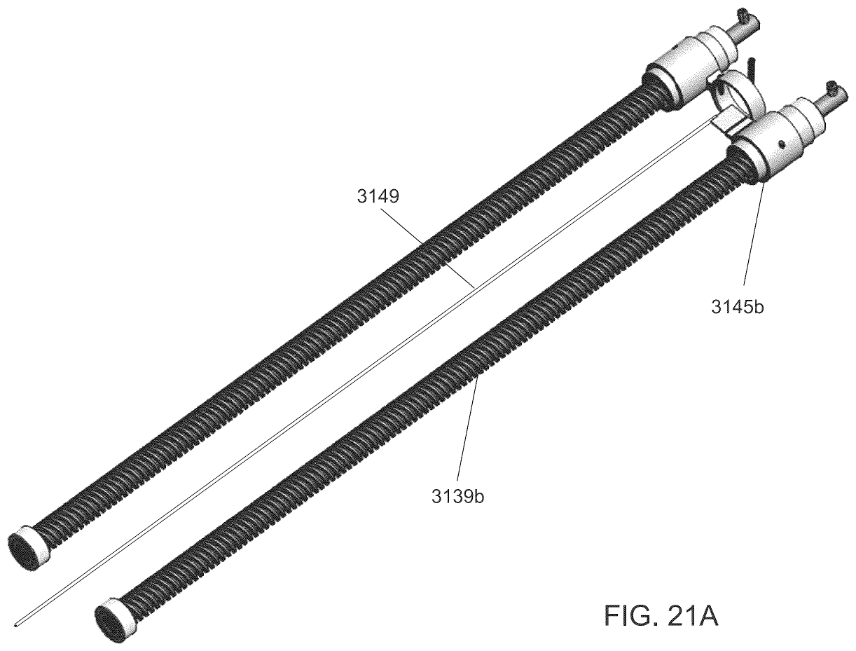

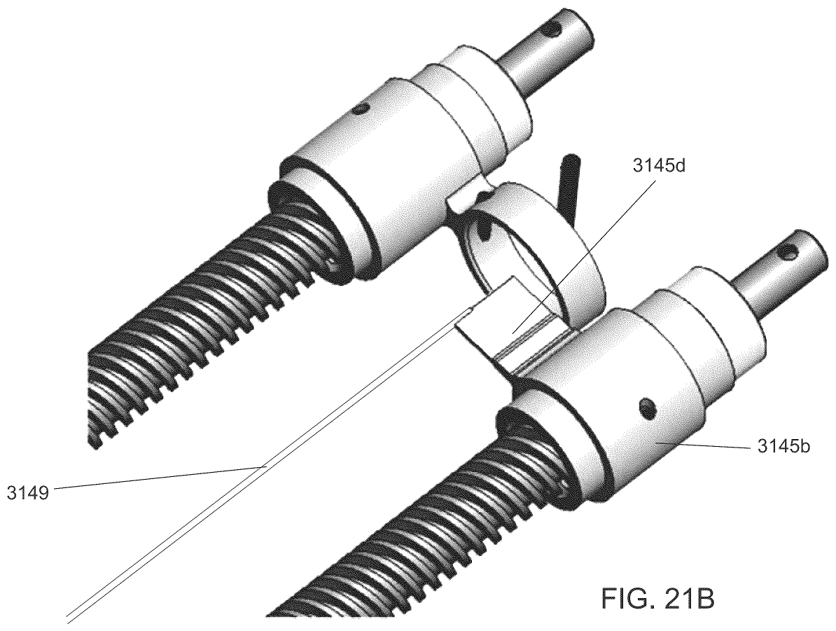

FIG. 21A illustrates the cartridge assembly 3140 with the outer housing removed and with the catheter guide element 3144 and the needle guide element 3146 removed, such that the guidewire 3149 and the coupling between the guidewire 3149 and the guidewire guide element 3145b (structurally and/or functionally similar to guidewire guide 544c) is viewable, in accordance with some embodiments. The guidewire 3149 is coupled to the guidewire guide element 3145b such that the guidewire 3149 translates in tandem with the guidewire guide element 3145b when the screw shaft 3139b rotates. FIG. 21B provides a close-up view of the engagement between the guidewire guide element 3145b and the guidewire 3149. The guidewire guide 3145 may be coupled to the guidewire 3139 via a press-fit, a slot, a fastener, adhesive, or the like.

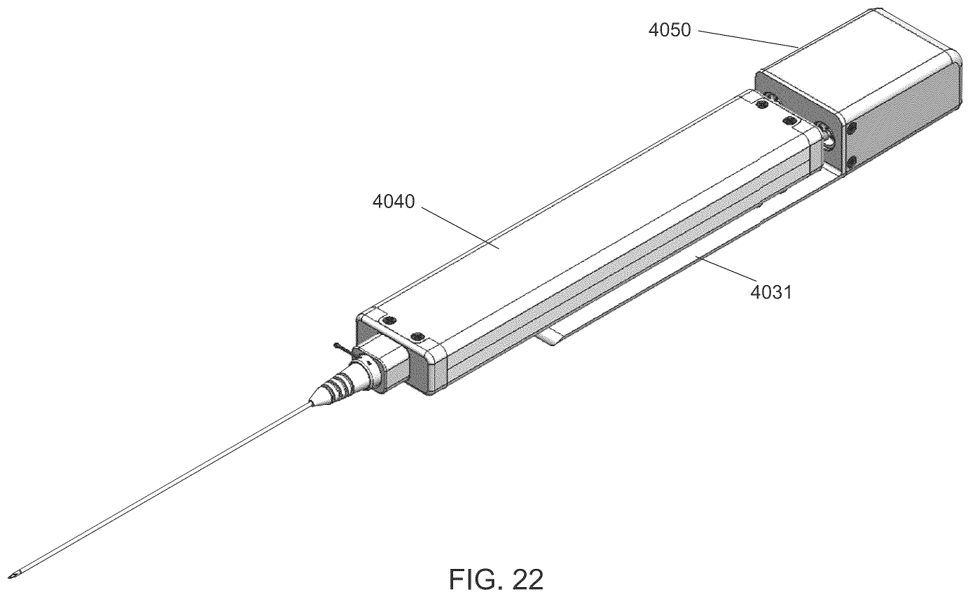

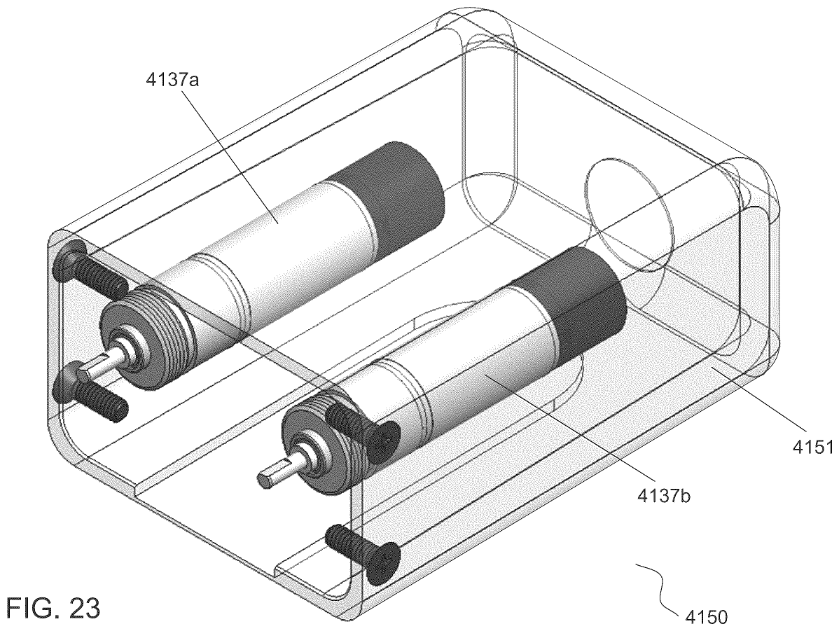

FIG. 22 illustrates a cartridge assembly 4040 (e.g., structurally and/or functionally similar to the other cartridge described herein, including the cartridge assembly 240 in FIG. 2 and/or the cartridge assembly 3040 in FIG. 11 and/or the cartridge assembly 3140 of FIG. 13) operably coupled to a motor system 4050, in accordance with some embodiments. The cartridge assembly 4040 is operably coupled to the motor system 4050 such that motors (e.g., discussed further in reference to FIG. 23) of the motor system 4050 are engaged with the actuators (e.g., 3134a and 3134b of FIG. 18). The motors, when engaged with the actuators, drive the movement of a ball screw nut along the shaft of the actuators. The cartridge assembly 4040 and/or the motor system 4050 may be disposable, or certain portions of the cartridge assembly 4040 can be reusable after appropriate disinfection and sanitation procedures. The cartridge assembly 4040 can be sterile such that can be exposed to the patient and potential contaminants, while allowing for the other components of the robotic system, such as robotic system 202, to be shielded behind a drape.

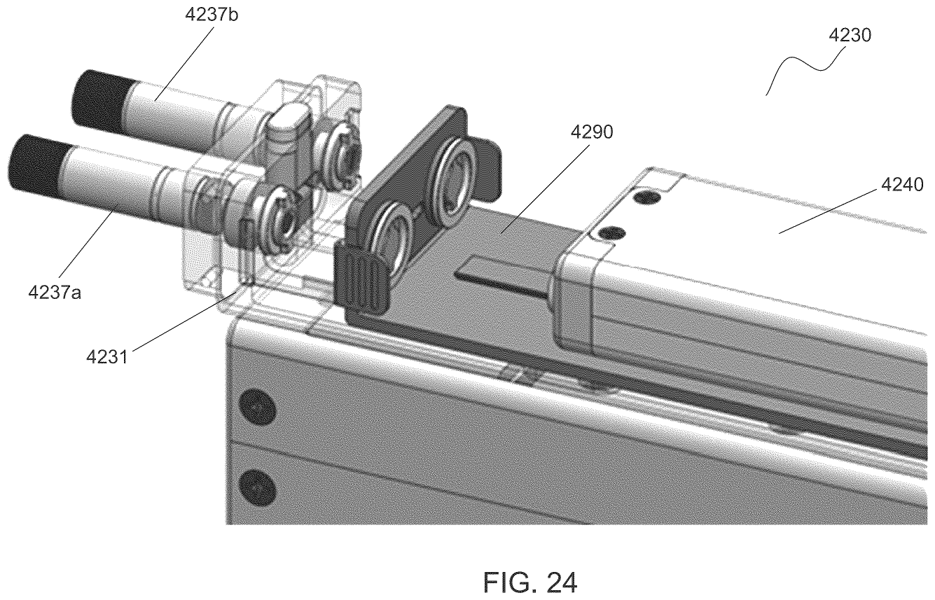

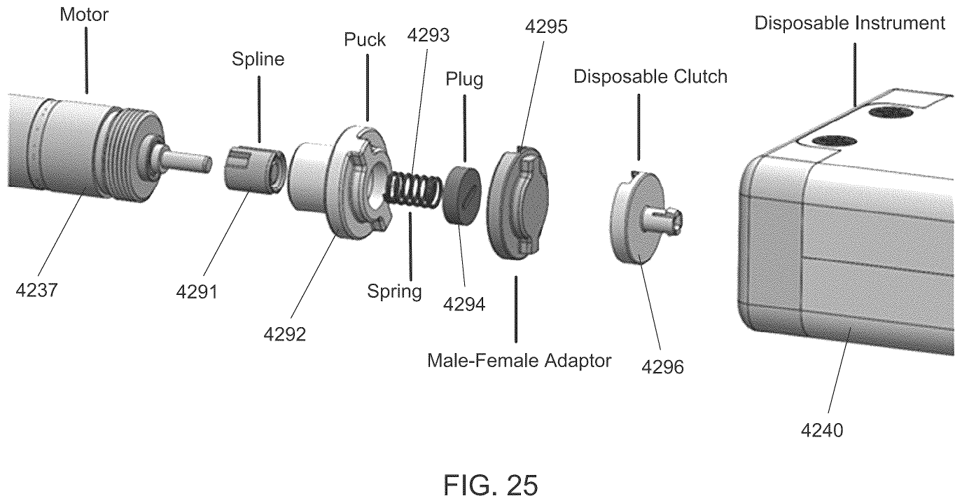

The motor assembly 4050 is coupled to an interface plate 4031. The interface plate 4031 couples to a linear actuator (e.g., such as the linear actuator 4634 of FIG. 28) and allows for the motor assembly 4050 and the cartridge assembly 4040 to translate. The interface plate 4031 includes apertures through which the motors and/or a motor interface assembly (e.g., the clutch system described in reference to FIGS. 25-27E). In some embodiments, the cartridge assembly 4040 is coupled directly to the interface plate 4031. In some embodiments, the cartridge assembly 4040 is coupled to the interface plate 4031 via an adaptor (e.g., the adaptor 4290 of FIG. 24).

FIG. 23 illustrates a component view of a motor assembly 4150 (e.g., structurally and/or functionally similar to the motor assembly 4050), in accordance with some embodiments. The motor assembly 4150 include a first motor 4137a and a second motor 4137b housed within a motor housing 4151. The motor housing 4151 may be configured to protect the first motor 4137a and the second motor 4137b from contaminants. The motor housing 4151 may include openings and/or apertures for wiring that provides the first motor 4137a and the second motor 4137b with signals and power for operation. In some embodiments, the motor housing 4151 includes features configured to provide the first motor 4137a and the second motor 4137b with structural support. For example, the housing 4151 may include shafts into which the first motor 4137a and the second motor 4137b are inserted. In some embodiments, the first motor 4137a and the second motor 4137b are coupled to the housing 4151 via a fastener (e.g., clip, screw, bolt, etc.). In some embodiments, the first motor 4137a and the second motor 4137b are coupled to the housing 4151 via an adhesive (e.g., glue, epoxy, etc.) or a weld. In some embodiments, the first motor 4137a and the second motor 4137b may be removeable. For example, the first motor 4137a and the second motor 4137b may be removed to be replaced, cleaned, and/or repaired.

The first motor 4137a and the second motor 4137b are configured to operably couple to linear actuators (e.g., the linear actuators 3134a and 3134b of the cartridge 3140 of FIG. 17). In some embodiments, the first motor 4137a is coupled to a linear actuator configured to translate a catheter (e.g., the linear actuator 3134a of the FIG. 17) and the second motor 4137b is coupled to a linear actuator configured to translate a guidewire (e.g., the linear actuator 3134b of FIG. 17). The first motor 4137a and the second motor 4137b may be a rotational motor such as a brushless DC motor. In some embodiments, the motor assembly 4150 may include a power source (e.g., battery, etc.) that provides power to the first motor 4137a and the second motor 4137b. In some embodiments, the motor assembly 4150 may include a wireless communication device (e.g., Wi-Fi module, Bluetooth module, etc.) to facilitate wireless operation of the first motor 4137a and the second motor 4137b.

FIG. 24 illustrates an adaptor 4290 interfacing a cartridge 4240 (e.g., structurally and/or functionally similar to the cartridge 4040 of FIG. 22) and a set of motors 4237a and 4237b (e.g., structurally and/or functionally similar to the first motor 4137a and the second motor 4137b of FIG. 23), in accordance with some embodiments. The adaptor 4290 is located between the cartridge 4240 and an interface plate 4231 (e.g., structurally and/or functionally similar to the interface plate 4031 of FIG. 22). In some embodiments, the adaptor 4290 may selectively couple to the interface plate 4231. For example, the adaptor 4290 may slide onto or clip onto the interface plate 4231. In some embodiments, the adaptor 4290 is selectively coupled to the cartridge 4240. In some embodiments, the adaptor 4290 is selectively coupled to both the interface plate 4231 and the cartridge 4240, thus allowing for the cartridge 4240 to translate together with the interface plate 4231.

The adaptor 4290 includes apertures corresponding to the motors 4237a and 4237b. The apertures allow for the motors 4237a and the 4237b to engage with the cartridge 4240 and operate the components of the cartridge 4240. In some embodiments, the adaptor 4290 may include a clutch system (e.g., discussed further in reference to FIG. 25) located in the apertures configured to transfer mechanical energy from the motors 4237a and 4237b to the cartridge 4240 while shielding the motors 4237a and 4237b from contaminants. The adaptor 4290 may be used as an interface for attaching a drape to protect the components of a manipulation device 4230 (e.g., structurally and/or functionally similar to the manipulation device 130 of FIG. 1) from contamination. In some embodiments, the motors 4237a and 4237b and the adaptor 4290 are configured to engage different types of cartridges 4240. For example, the motors 4237a and 4237b and the adaptor 4290 are configured to selectively engage a cartridge 4240 configured to access a radial vein or a cartridge 4240 configured to access a jugular vein.

FIG. 25 illustrates a clutch system, in accordance with some embodiments. The clutch system is located between a motor 4237 (e.g., structurally and/or functionally similar to the motors 4137a and 4137b of FIG. 23) and a cartridge 4240 (e.g., structurally and/or functionally similar to the other cartridge described herein, including the cartridge assembly 240 in FIG. 2, and/or the cartridge assembly 3040 in FIG. 11, and/or the cartridge assembly 3140 of FIG. 13, and/or the cartridge assembly 4040 of FIG. 22). The clutch system is configured to engage and transfer mechanical energy from the motor 4237 to the cartridge 4240. The clutch system is also configured to maintain separation between the motor 4237 and the cartridge 4240, thus protecting the motor 4237 from contaminants. While FIG. 25 only shows one motor 4237 and a one clutch system, embodiments may include multiple motors and clutch systems.

The clutch system includes a spline 4291, a puck 4292, a spring 4293, a plug 4294, a male-female adaptor 4295, and a clutch 4296 arranged linearly from the motor 4237 to the cartridge 4240. In some embodiments, some, or all of the components of the clutch system 4291 may be disposable to prevent contamination. In some embodiments, only the clutch 4296 is disposable. The spline 4291 operably couples to a motor shaft of the motor 4237. In some embodiments, the spline 4291 may be coupled to the motor shaft via a fastener (e.g., clip, set screw, etc.), mechanically (e.g., slot, friction fit, etc.), welded, or via an adhesive (e.g., glue, epoxy, etc.). The spline 4291 engages and operably couples to a puck 4292. The puck 4292 includes a lumen that houses the spring 4293 and is covered by the plug 4294. The spring and the plug 4294 provide constant pressure on the male-female adaptor 4295 which selectively engages the puck 4292. The puck 4292 and the male-female adaptor include a corresponding extrusion pattern that, when engaged, allow the puck 4292 to rotate the male-female adaptor 4295. In some embodiments, the male-female adaptor is the final component of the clutch system between the motor 4237 and an adaptor, such as the adaptor 4290 of FIG. 25. The male-female adaptor 4295 engages the clutch 4296 which engages the cartridge 4240. The male-female adaptor 4295 and the clutch 4296 include a corresponding extrusion pattern that, when engaged, allow the male-female adaptor 4295 to rotate the clutch 4296. The clutch 4296 may include protrusions on a clutch shaft to operably couple the clutch 4296 to the cartridge 4240.

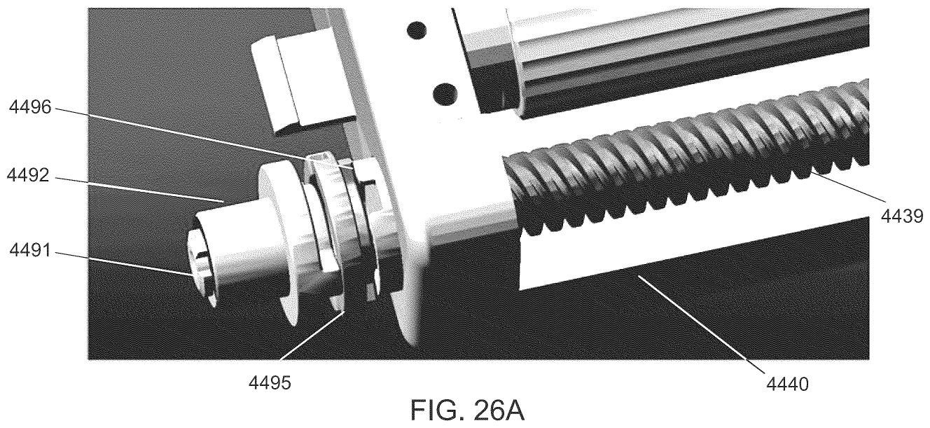

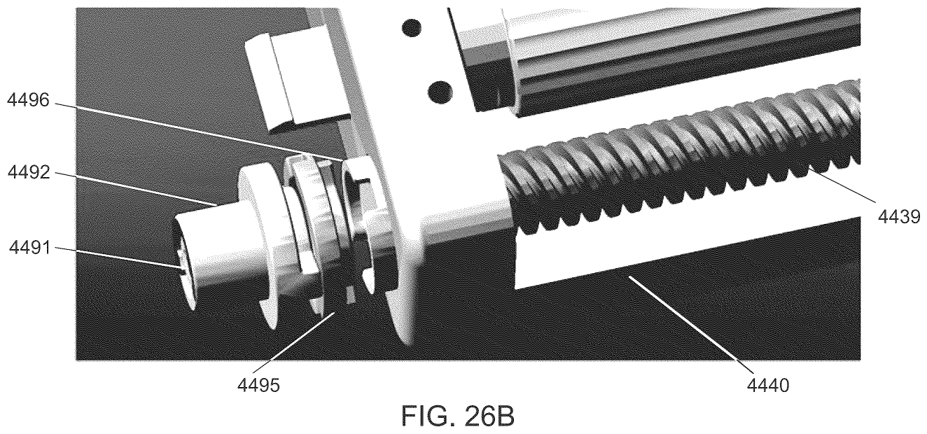

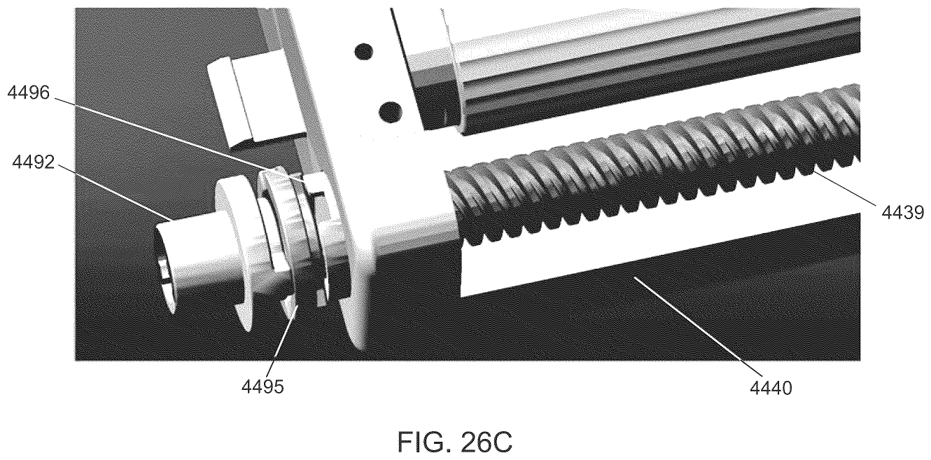

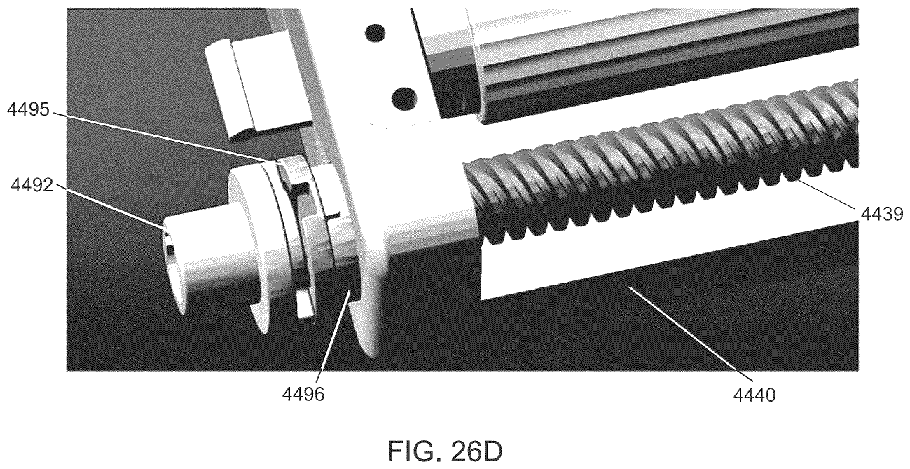

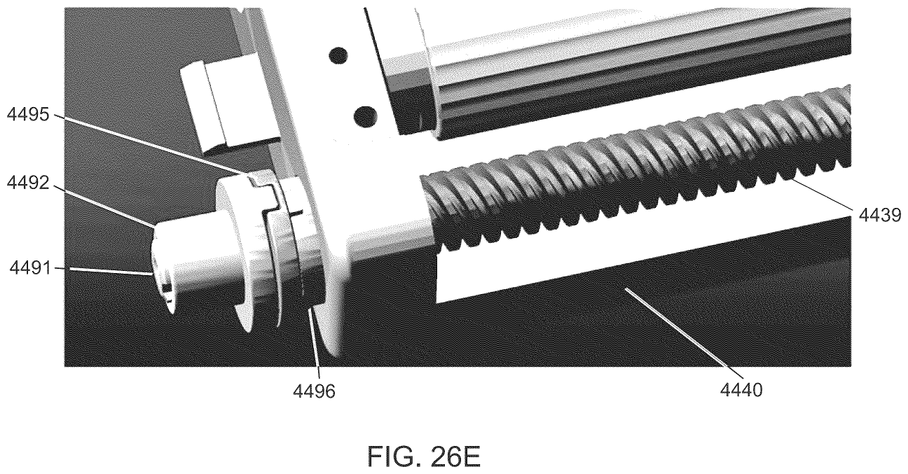

The clutch system allows for the clutch to be selectively engaged. The inclusion of the spring 4293 mechanism allows for the clutch system to operably couple the motor 4237 to the cartridge 4240 without adjusting orientations of the motor shaft or of the components of the cartridge 4240. The operation of the clutch system is described further in FIGS. 26A-26E.

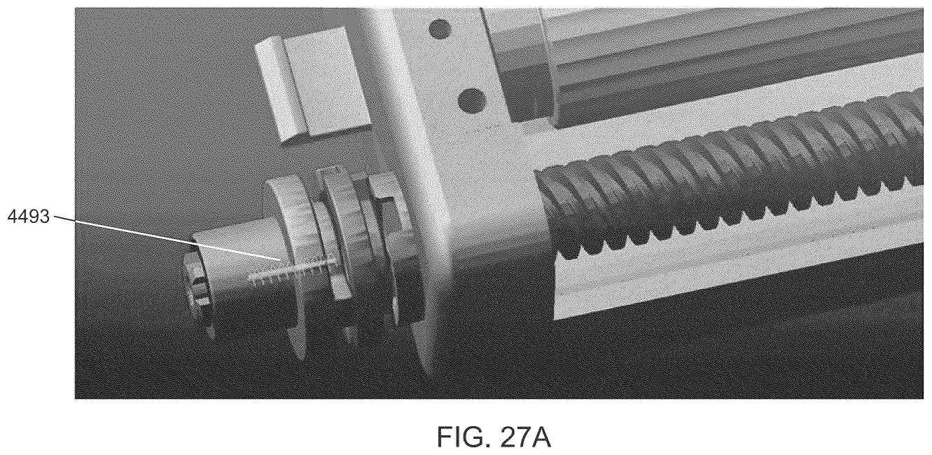

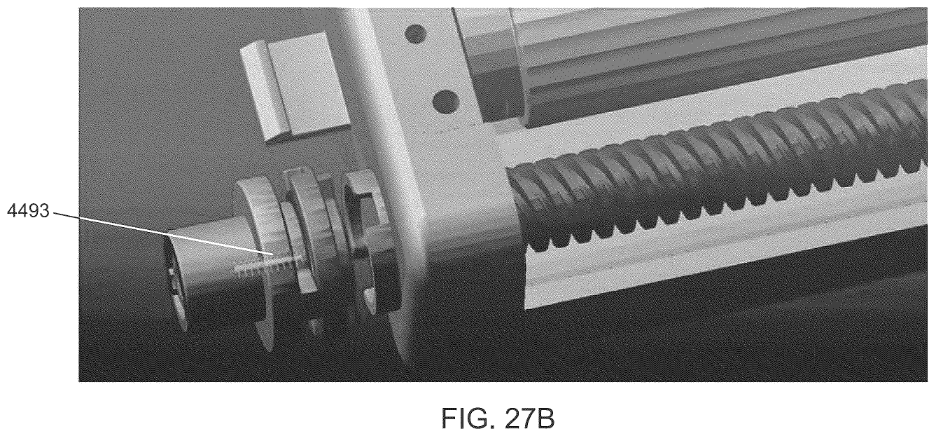

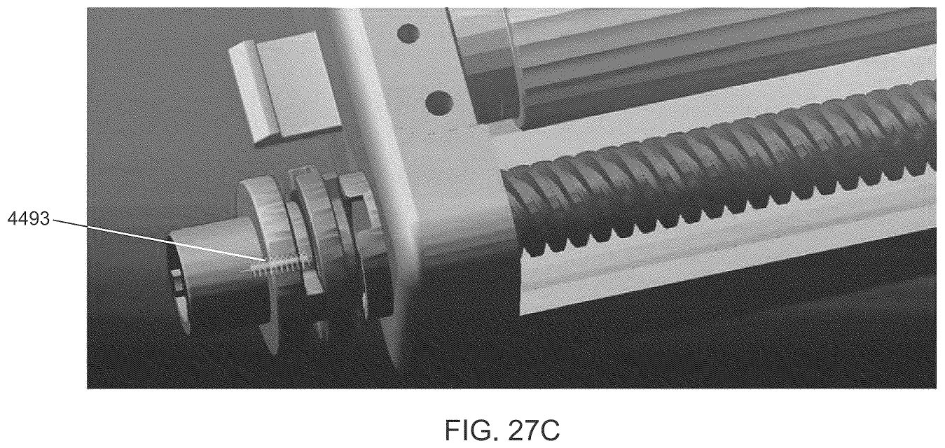

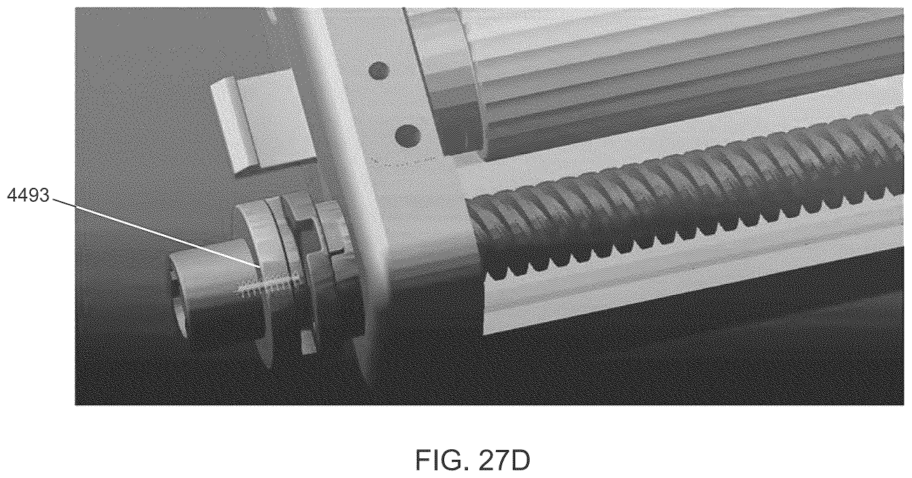

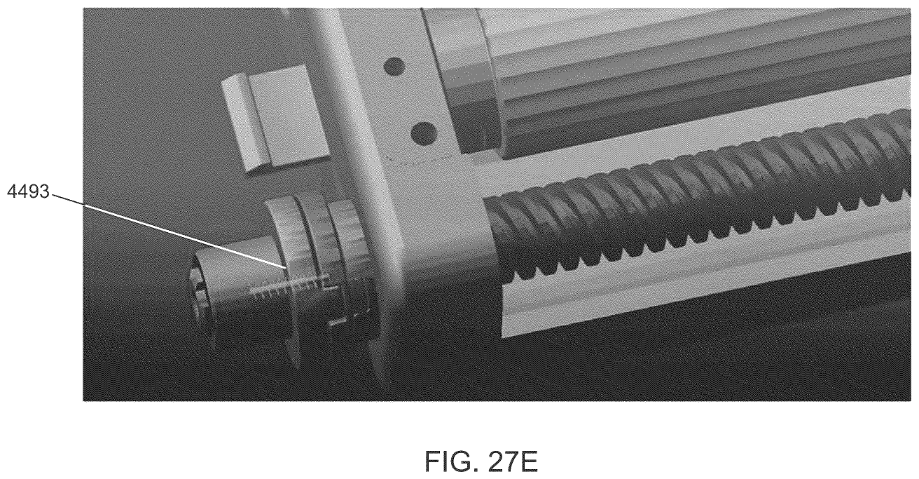

FIGS. 26A-26E illustrate a clutch system (e.g., structurally and/or functionally similar to the clutch system of FIG. 25) in operation, in accordance with some embodiments. FIG. 26A illustrates a clutch system operably disengaged from a linear actuator 4439 of a cartridge 4440 (e.g., structurally and/or functionally similar to the linear actuators 3134a and 3134b and the cartridge 3140 of FIG. 17). The clutch 4496 (e.g., structurally and/or functionally similar to the clutch 4296 of FIG. 25) is engaged to the cartridge 4440, but the male-female adaptor 4495 (e.g., structurally and/or functionally similar to the male-female adaptor 4295 of FIG. 25) is disengaged (e.g., unable to transfer mechanical energy) from the clutch 4496. The puck 4492 (e.g., structurally and/or functionally similar to the puck 4292 of FIG. 25) is also disengaged from the male-female adaptor 4495. The spline 4491 extends past the lumen of the puck 4492 indicating that the spline 4491 is not exerting pressure on the spring within the lumen of the puck 4492. The spline 4491 is engaged in FIG. 27B, thus beginning to exert pressure on the male-female adaptor 4495, which is advanced toward the clutch 4496 in FIG. 26C. The male-female adaptor 4495 is advanced toward the clutch 4496 until the male-female adaptor 4495 and the clutch 4496 are in contact but may not be engaged yet.

Once the male-female adaptor 4495 and the clutch 4496 are in contact, the male-female adaptor 4495 is rotated by a motor (not shown) until the male-female adaptor 4495 and the clutch 4496 are engaged, as seen in FIG. 26D. Once engaged, the motor rotates puck 4492 until the puck 4492 is engaged with the male-female adaptor 4495, as ween in FIG. 26E. Once the clutch system is fully engaged, the spline 4491 does not exert a pressure force on the spring. Once fully engaged, the clutch system allows for mechanical energy to be transferred from the motor directly to the linear actuator 4439 of the cartridge 4440. The mechanism of the clutch system allows for the cartridge 4440 to be operably coupled to a motor without adjusting exact positioning prior to coupling, thus increasing efficiency, and decreasing workload.

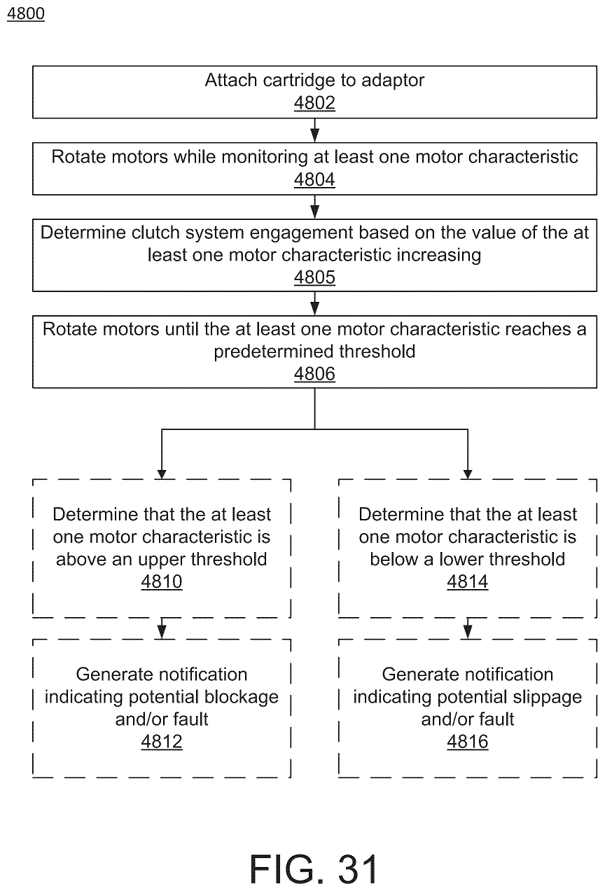

FIG. 31 is a flow diagram of a method 4800 of engaging and monitoring a clutch system (e.g., structurally and/or functionally similar to the clutch system of FIG. 25 and/or the clutch system of FIGS. 26A-26E). The engagement of the clutch system may be monitored via a control unit (e.g., functionally and/or structurally similar to the control unit 405 of FIG. 4). The control unit may actuate motors (e.g., functionally and/or structurally similar to the motor 4237 of FIG. 25) and/or receive information from the motors themselves and/or sensors (e.g., torque sensor, ammeter, power meter, strain gauge, etc.) configured to monitor the operation of the motors.

At 4802, a cartridge (e.g., functionally and/or structurally similar to the cartridge 4240 of FIG. 24) is attached to an adaptor (e.g., functionally and/or structurally similar to the adaptor 4290 of FIG. 24). In some embodiments, the cartridge clicks into place. In some embodiments, the cartridge may be attached to the adaptor via a fastener (e.g., pin, bolt, clip, screw, etc.). The cartridge is attached to the adaptor such that the clutch system is located between the motors and cartridge. Once the cartridge is attached, at 4804, the control unit rotates the motors while monitoring at least one motor characteristic. The control unit may rotate the motors either clockwise and/or counterclockwise depending on the orientation of the linear actuators within the cartridge. The at least one motor characteristic may be a torque output by the motors, current draw, power draw, strain, and/or another characteristic indicating the operation of the motors. The at least one motor characteristic may be measured by sensor(s) communicatively coupled to the control units.

At 4805, the control unit determines that the clutch system is engaged based on assessing the value of the at least one motor characteristic. For example, an increase in the value of the at least one motor characteristic may indicate that the clutch system is engaged with and transferring power to the linear actuators disposed in the cartridge, thus requiring a greater output from the motor. In some embodiments, the control unit may determine clutch system engagement based on the torque output of the motor increasing. In some embodiments, the control unit may determine clutch engagement based on the current draw of the motor increasing. In some embodiments, the control unit may determine clutch system engagement based on power draw of the motor increasing. In some embodiments, an increase greater than a predefined amount or predefined percentage may indicate that the clutch system has engaged with the linear actuators disposed in the cartridge.

At 4806, the control unit may rotate the motors in a direction that moves the linear actuators toward a position x=0 or actuator zero-position. For example, as described above, a linear actuator can include a screw shaft and a screw nut which linearly advances along the screw shaft. The control unit can control the motor to rotate the screw shaft such that the screw nut returns to a starting position, such as, for example, a position in which the screw nut bottoms out against a surface. The control unit can rotate the motors until at least one motor characteristic reaches a predetermined threshold (or first threshold). The predetermined threshold corresponds to a component (e.g., guidewire guide or associated screw nut, catheter guide or associated screw nut, etc.) of the cartridge contacting a limiting feature (e.g., the cartridge housing or other limiting surface). In response to a value of the at least one motor characteristic reaching a predetermined threshold, the control unit can determine or establish that the linear actuators are in the actuator zero-position. This position can indicate that the linear actuators are in their starting position, e.g., where the guidewire, catheter, and/or needle are fully retracted to a starting position. In some embodiments, 404-406 may be performed for a plurality of motors being coupled to linear actuators at the same time or concurrently. For example, a motor for driving movement of a guidewire and a motor for driving movement of a catheter may be rotated at the same time to engage their respective clutch systems with the linear actuators associated with the guidewire or motor, respectively, and to position the linear actuators in their starting position or actuator zero-position. Alternatively, 404-406 may be performed for different motors at different times or sequentially. For example, the motor for driving movement of the guidewire may be driven first to engage that motor with the linear actuator associated with the guidewire, and then the motor for driving the movement of the catheter may be driven to engage that motor with the linear actuator associated with the catheter. Setting the linear actuators to their zero-point position allows the control unit to confirm the precise location of the guidewire, needle, and/or catheter at the start of the vascular access operation and to ensure that each is accurately and precisely position during the operation.

In some embodiments, the control unit may optionally continue to monitor the at least one motor characteristic during operation to determine if the manipulator, clutch system, and/or cartridge are operating irregularly. For example, at 4810, the control unit can determine that the at least one motor characteristic exceeds an upper threshold (or second threshold). The at least one motor characteristic exceeding the upper threshold may indicate that some portion of the system has become blocked, such as, for example, the linear actuator being blocked from rotating, or that the cartridge and/or adaptor is faulty. In some embodiments, the control unit may determine that there is a potential blockage or that the cartridge and/or adaptor may be faulty in response to the at least one motor characteristic exceeding the upper threshold for a predetermined number of times (e.g., when there has been multiple attempts or multiple incidents of a measured motor torque being above the upper threshold) and/or the least one motor characteristic exceeding the upper threshold for a predetermined period of time (e.g., when the measured motor torque consistently stays above the upper threshold for a long period of time). Once the control unit determines a potential blockage or fault, the control unit generates a notification communicating the potential blockage or fault, at 4812.

As another example, at 4814, the control unit can determine that the at least one motor characteristic is below a lower threshold (or third threshold). The at least one motor characteristic being below a lower threshold may indicate that the clutch system is slipping (e.g., detaching from the linear actuators) or that the cartridge and/or adaptor is faulty. In some embodiments, the control unit may determine that there is a potential slippage or that the cartridge and/or adaptor may be faulty in response to the at least one motor characteristic being below the lower threshold for a predetermined number of times (e.g., when there has been multiple attempts or multiple incidents of a measured motor torque being below the lower threshold) and/or the least one motor characteristic being below the lower threshold for a predetermined amount of time (e.g., when the measured motor torque consistently stays below the lower threshold for a long period of time). Once the control unit determines a potential slippage or fault, the control unit generates a notification communicating the potential slippage or fault, at 4816.

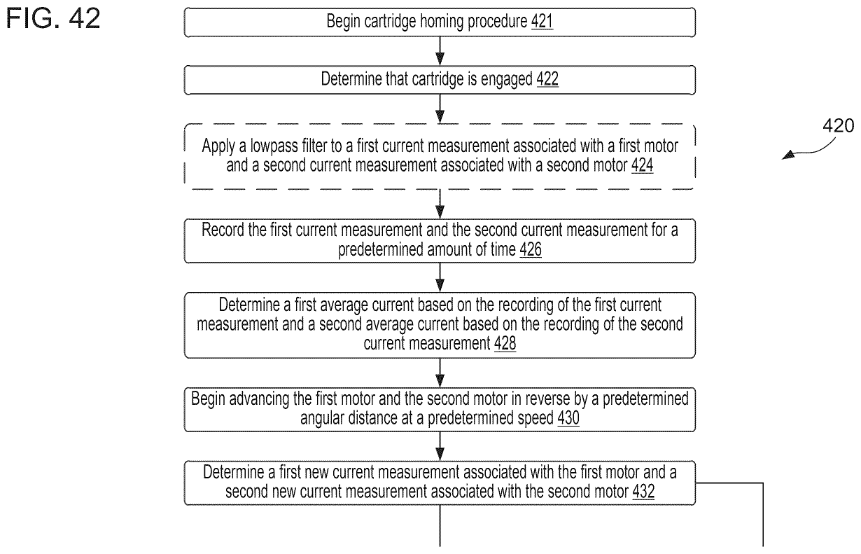

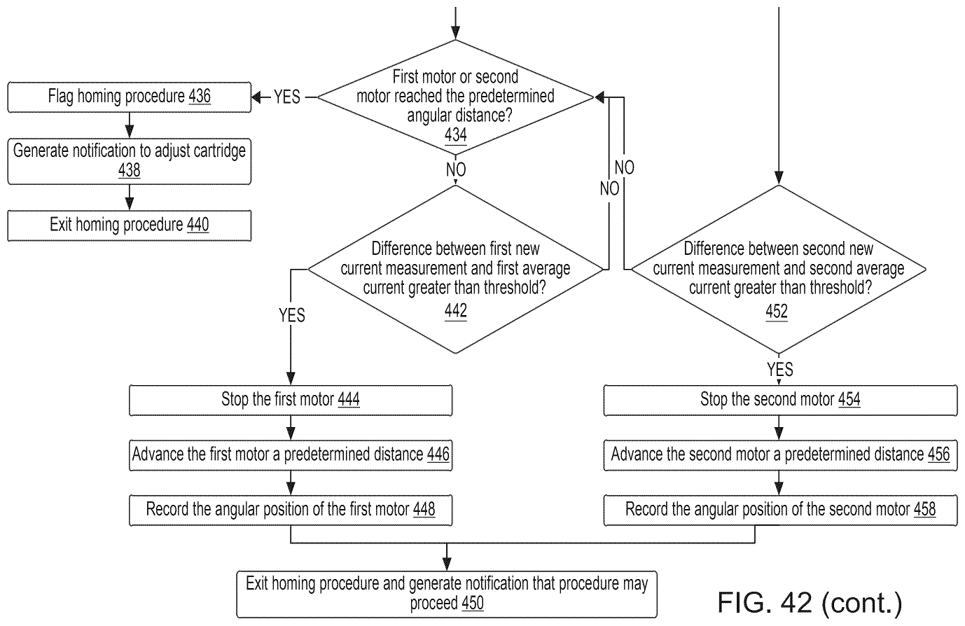

FIG. 42 is a flow diagram illustrating a method 420 of a homing procedure (e.g., cartridge homing procedure) for a manipulation device, in accordance with some embodiments. The homing procedure allows for homing (e.g., zeroing, setting to a zero position, setting to a known position, etc.) the actuators (e.g., structurally and/or functionally similar to nay of the actuators described herein, such as the device actuator(s) 534 of FIG. 5) of a cartridge (e.g., functionally and/or structurally similar to any of the cartridges described herein, such as the cartridge assembly 540 of FIG. 5). The homing procedure can allow for determining if the cartridge is properly coupled to the motors (e.g., functionally and/or structurally similar to any of the motors described herein, such as the motors 4137a and/or 4137b of FIG. 23). In some embodiments, the homing procedure is used after coupling the cartridge to the manipulation device (e.g., structurally and/or functionally similar to any of the manipulation devices described herein, such as the manipulation device 530 of FIG. 5), prior to a procedure to prepare the cartridge for use during the procedure. In some embodiments, the homing procedure may be executed by a control unit (e.g., functionally and/or structurally similar to the control unit 405 of FIG. 4). The control unit may actuate the motors and/or receive information from the motors themselves and/or sensors (e.g., torque sensor, ammeter, power meter, strain gauge, etc.) configured to monitor the operation of the motors.

At 421, the control unit begins the cartridge homing procedure. In some embodiments, the cartridge homing procedure begins in response to the control unit receiving an input that the cartridge is coupled, in response to the control unit receiving an input from a user, and/or the like. At 422, the control unit determines that the cartridge is engaged. In some embodiments, the cartridge may include a tag (e.g., Bluetooth, near-field communication tag, etc.) that the control unit can used to determine information about the cartridge and/or if the cartridge is engaged to the manipulation device. In some embodiments, the control unit can determine that the cartridge is engaged based on a sensor signal, such as a signal from a pressure sensor, position, sensor, and/or the like.

At 424, the control unit optionally applies a lowpass filter to a first current measurement associated with a first motor and a second current measurement associated with a second motor. The first current measurement and/or the second current measurements can be received by the control unit from one or more ammeter configured to measure the current draw from the first motor and/or the second motor. In some embodiments, the first motor is coupled to a linear actuator configured to translate a catheter (e.g., structurally and/or functionally similar to the linear actuator 3134a of the FIG. 17) and the second motor is coupled to a linear actuator configured to translate a guidewire (e.g., structurally and/or functionally similar to the linear actuator 3134b of FIG. 17). In some embodiments, the control unit may receive the first current measurement and/or the second current measurement and not apply a filter.

At 426, the control unit records the first current measurement and the second current measurement for a predetermined amount of time. In some embodiments, the predetermined amount of time is about 2 seconds. In some embodiments, the predetermined amount of time can be about 0.1 second, about 0.2 seconds, about 0.5 seconds, about 1 second, about 2 seconds, about 3 seconds, about 5 seconds, about 10 seconds, inclusive of any ranges and values therebetween. At 428, the control unit determines a first average current based on the first current measurement and a second average current based on the recording of the second current measurement.

At 430, the control unit commands the first motor and the second motor to operate in reverse (e.g., such that the linear actuator turns backwards) by a predetermined angular distance at a predetermined speed. In some embodiments, the predetermined angular distance is about 720 degrees. In some embodiments, the predetermined speed is 180 degrees per second. In some embodiments, the predetermined speed and/or the predetermine angular distance can be determined based on the type of motor, cartridge, and/or the like. At 432, the control unit determined a first new current measurement associated with the first motor and a second new current measurement associated with the second motor while the first motor and the second motor are operating as described in reference to 430. In some embodiments, the first new current measurement and/or the second new current measurement can be an average current over a predetermined period of time. After 432, the method 420 continues to 434 and to 452.

At 434, the control unit determines if the first motor or the second motor reaches the predetermined angular distance. If the control unit determines that the first motor and/or the second motor reached the predetermined angular distance, the method 420 continues to 436. The first motor and/or the second motor reaching the predetermined angular distance can indicate that the cartridge not engaged properly. For example, the cartridge may not have engaged the motors, or the cartridge may have had the catheter or the guidewire in an extended position during engagement with the manipulation device. At 436, the control unit flags the homing procedure 436. Flagging the homing procedure can include generating a notification to a user that the homing procedure was not successful. At 438, the control unit generates a notification to the user to adjust the cartridge. In some embodiments, the notification can include information about how to adjust the cartridge. For example, the notification can include information to adjust an actuator. At 440, the control unit exits the homing procedure. In some embodiments, the method 402 can return to 421 after the user adjusts the cartridge as indicated in the notification generated in 438.

If the control unit, at 434, determines that the first motor and the second motor do not reach the predetermined angular distance, the method 420 continues to 442. At 442, the control unit determines if the difference between the first new current measurement and the first average current is greater than a threshold. The threshold can be a predetermined threshold that corresponds to a current difference that indicates that the motor has reached a zero position. If difference is not greater than the threshold, the method 420 returns to 434. If the control unit determines that the difference is greater, the control unit stops the first motor at 444 and advances the first motor a predetermined distance at 446. The predetermined distance can correspond to a linear distance the linear actuator can translate. For example, the linear distance may be about 0.1 mm. Advancing the first motor by the predetermined distance allows for the motor to be in a home position that is prepared for a procedure. At 448, the control unit records the angular position of the first motor, where the angular position of the first motor corresponds to the home position of the first motor.

At 452, the control unit determines if the difference between the second new current measurement and the second average current is greater than a threshold. The threshold can be a predetermined threshold that corresponds to a current difference that indicates that the motor has reached a zero position. If the difference is not greater than the threshold, the method 420 returns to 434. If the control unit determines that the difference is greater, the control unit stops the second motor at 454 and advances the first motor a predetermined distance at 456. The predetermined distance can correspond to a linear distance the linear actuator can translate. For example, the linear distance may be about 0.1 mm. Advancing the first motor by the predetermined distance allows for the motor to be in a home position that is prepared for a procedure. At 458, the control unit records the angular position of the first motor, where the angular position of the first motor corresponds to the home position of the first motor. After 448 and 458 are executed by the control unit, the method 420 proceeds to 450. At 450, the control unit exits the homing procedure and generates a notification to the user that the procedure may proceed.

FIGS. 27A-27E illustrate a spring 4493 (e.g., structurally and/or functionally similar to the spring 4293 of FIG. 25) of a clutch system in operation, in accordance with some embodiments. FIGS. 27A-27E correspond to FIGS. 26A-26E. For example, FIG. 27A depicts the state of the spring within the clutch system of FIG. 26A, FIG. 27B depicts the state of the spring within the clutch system of FIG. 26B, FIG. 27C depicts the state of the spring within the clutch system of FIG. 26C, FIG. 27D depicts the state of the spring within the clutch system of FIG. 26D, and FIG. 27E depicts the state of the spring within the clutch system of FIG. 26D.

In FIG. 27A, as the clutch system is disengaged, the spring 4493 is decompressed as the clutch system is not actively engaging the cartridge. In FIG. 27B, as the male-female adaptor is moved toward the clutch, the spring 4493 begins to be compressed and is fully compressed, as seen in FIG. 27C, as the male-female adaptor is in contact with the clutch. The spring 4492 remains compressed, as seen in FIG. 27D as male-female adaptor engages the clutch, but decompresses, as seen in FIG. 27E, when the clutch system is fully engaged.

FIG. 28 illustrates a translation assembly 4680 of a manipulation device 4630 coupled to a cartridge 4640 (e.g., structurally and/or functionally similar to the other cartridges described herein, including the cartridge assembly 240 in FIG. 2, and/or the cartridge assembly 3040 in FIG. 11, and/or the cartridge assembly 3140 of FIG. 13, and/or the cartridge assembly 4040 of FIG. 22, and/or the cartridge assembly 4240 of FIG. 25) and a motor system 4650 (e.g., functionally and/or structurally similar to the motor assemblies described herein, including the motor assembly 4050 of FIG. 22, and/or the motor assembly 4150 of FIG. 23) in accordance with some embodiments. The translation assembly 4680 is housed within a housing of the manipulation device 4630 (not shown). The cartridge 4640 and the motor assembly 4650 are coupled to an interface plate 4631 (e.g., structurally and/or functionally similar to interface plate 4031 of FIG. 22) such that the cartridge 4640 and the motor assembly 4650 move together with the interface plate 4631. The interface plate 4631 is coupled to the translation assembly 4680 which is configured to translate the interface plate 4631, and thus the cartridge 3640 and the motor assembly 4650, along an axis. The translation assembly 3680 allows for the needle of the cartridge 4640 to be positioned relative to the manipulation device 4630.

FIG. 29 illustrates the translation assembly 4680 of FIG. 28, in accordance with some embodiments. The translation assembly 4680 is configured to translate the interface plate 4631, as well as any components coupled to the interface plate 4631, such as the motor assembly 4650 and a cartridge. The translation assembly 4680 includes a linear actuator 4634 operably coupled to a linear guide 4644a, which is operably coupled to a rotational guide 4644b. The linear actuator 4634 include a motor 4637, a linear shaft 4639, a front plate 4661a, and a backplate 4661b.

The linear shaft 4639 is mounted between the front plate 4611a and the backplate 4661b. The linear shaft 4639 is a ball shaft supported by ball bearings within the front plate 4661a and the back plate 4661b. The front plate 4611a and the back plate 4611b are fixedly coupled to the housing of the manipulation device and lock the linear shaft 4639 into a constant axial position. The motor 4637 is mounted in the front plate 4661a and couples to the linear shaft 4639. The motor drives the movement of the ball screw nut along the line shaft 4639. The motor 4637 may be any type of electric motor, such as a brushless DC motor. When activated, the motor 4637 rotates, thus rotating the linear shaft 4639. The linear guide 4644a is operably coupled to the linear shaft 4639 such that the ball screw nut within the linear guide 4644a translates axially along the linear shaft 4639 as the linear shaft 4639 is rotated by the motor 4637. The linear guide 4644a is fixedly coupled to the interface plate 4631. In some embodiments, the linear guide 4644a is coupled to the interface plate 4631 via a fastener (e.g., screw, bolt, etc.). The rotational guide 4644b is fixedly coupled to the front plate 4661a and the backplate 4661b to prevent the linear guide 4644a from rotating during operation and results in the interface plate 4631 translating axially along the length of the linear shaft 4639. In some embodiments, the rotational guide 4644b may include at least one stopper that may limit the range of motion of the interface plate 4631.

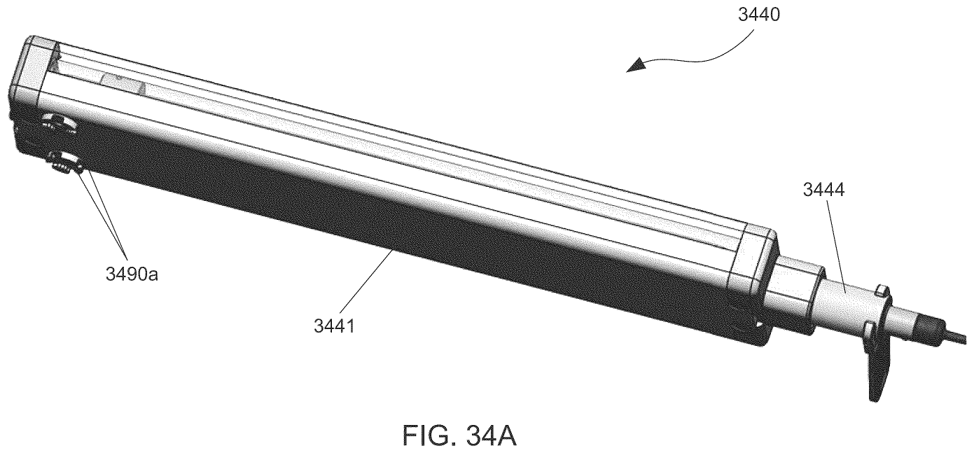

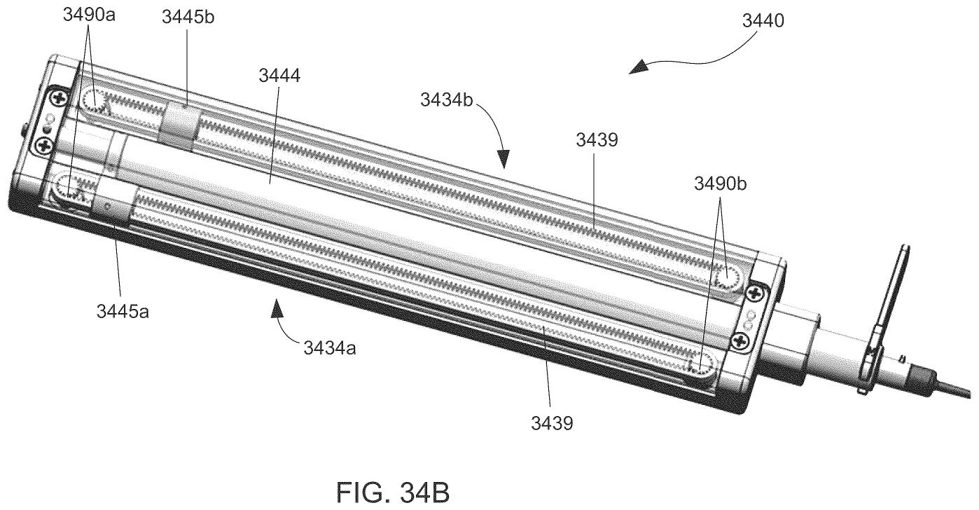

FIG. 34A illustrates another example of a cartridge 3440, in accordance with some embodiments. In some embodiments, the cartridge 3440 is structurally and/or functionally similar to the other cartridges described herein, including the cartridge assembly 240 in FIG. 2, and/or the cartridge assembly 3040 in FIG. 11, and/or the cartridge assembly 3140 of FIG. 13, and/or the cartridge assembly 4040 of FIG. 22, and/or the cartridge assembly 4240 of FIG. 25, and/or the cartridge 4640 of FIG. 28. The cartridge 3440 includes a cartridge body 3441 that houses the components of the cartridge 3440. A catheter guide 3444 extends away from the cartridge body 3441. The cartridge 3440 includes clutch drives 3490a disposed on the cartridge body 3441. The clutch drives 3490a are located on a bottom face of the cartridge body 3441 corresponding to the side that engages a manipulation device (e.g., functionally and/or structurally similar to the manipulation device 3060 of FIG. 11) opposite of the catheter guide 3444. The clutch drives 3490a can be engaged by an adapter on a manipulation device with motors configured to engage each clutch drive 3490a. Including the clutch drives on the bottom face of the cartridge body 3441 allows for the cartridge to be pressed into place on the adapter, which may be more user-friendly and facilitate engaging the cartridge with the manipulation device. Furthermore, this method of engagement allows for draping in a single plane.

FIG. 34B illustrates the cartridge 3440 of FIG. 34A with internal components shown. The cartridge 3440 includes a catheter actuator 3434a and a guidewire actuator 3434b. Each of the actuators 3434a, 3434b includes a clutch drive 3490a and a tensioning element 3490b operably coupled together via a belt 3439. The tensioning elements are located on the distal side of the cartridge 3440. The catheter actuator 3434a further includes a catheter guide element 3445a operably coupled to an associated belt 3439, the catheter guide element 3445a being configured to translate the catheter guide 3444. The guidewire actuator 3434b further includes a guidewire guide element 3445b operably coupled to an associated belt 3439, the guidewire guide element 3445b configured to translate a guidewire.

During operation, the clutch drives 3490a rotate and cause the belt 3439 to translate the guide elements 3445a, 3445b. In some embodiments, the guide elements 3445a, 3445b are anchored to the respective belt 3439. The tensioning elements 3490b are pulleys that provide the belt 3439 with tension, e.g., in order to reduce lash. The tensioning elements 3490b can be part of a tensioning system that is molded into the catheter body 3441. The tensioning system can include a ratcheting system that creates tension on the belt during assembly.

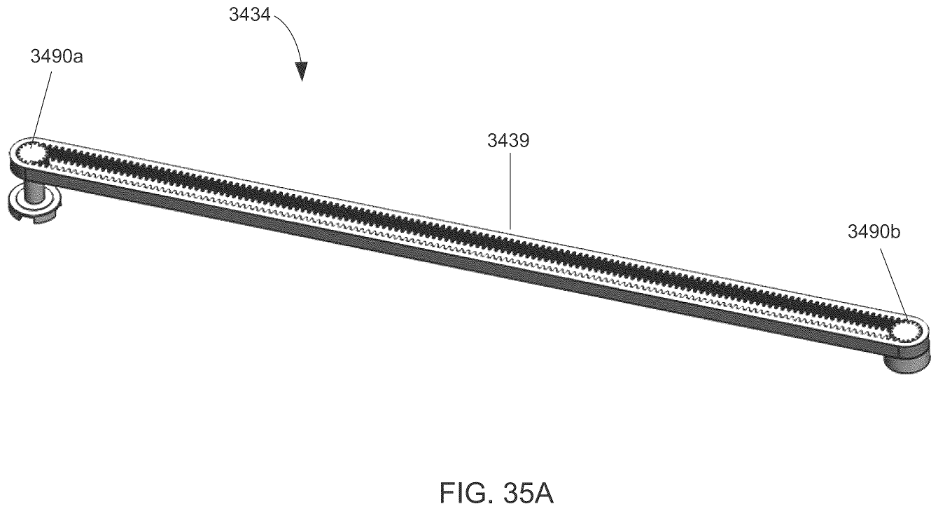

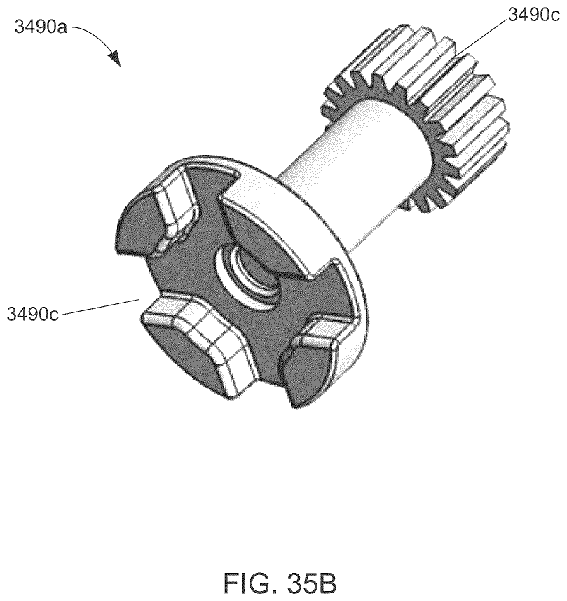

FIG. 35A illustrates a belt actuator 3434 (e.g., functionally and/or structurally similar to the actuator 3434a, 3434b of FIG. 34B), in accordance with some embodiments. The belt actuator 3434 includes a clutch drive 3490a and a tensioning element 3490b operably coupled together via a belt 3439. The clutch drive 3490a, as seen in FIG. 35B, includes a motor engagement portion 3490c configured to engage with a motor that rotates the clutch drive. The clutch drive 3490a further includes a toothed portion 3490 which is configured to engaged with teeth in the belt 3439, causing the belt 3439 to rotate about the clutch drive 3490a and the tensioning element 3490b. In some embodiments, the belt 3439 is formed of rubber or another elastic material.

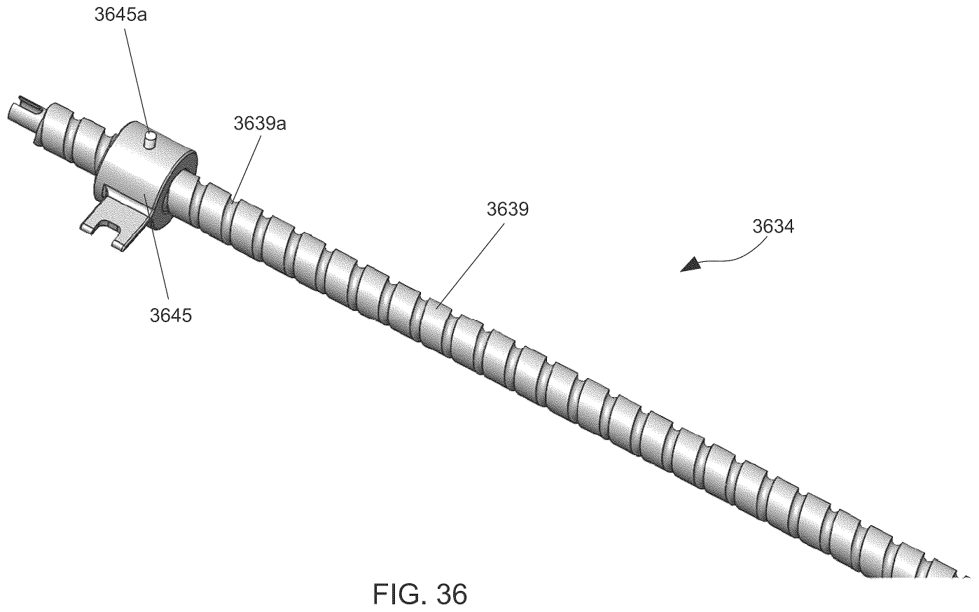

FIG. 36 illustrates a portion of an actuator 3634 e.g., structurally and/or functionally similar to device actuator(s) 534 of FIG. 5, in accordance with some embodiments. The actuator 3634 includes a driven element 3645 (e.g., guide element) operably coupled to a shaft or cam 3639. The shaft 3639 includes a groove 3639a that spirals around the length of the shaft 3639. The driven element 3645 includes a follower 3645a (e.g., pin, screw, etc.) that fits into the groove 3639a such that the driven element 3645 translates along the length of the shaft 3639 as the shaft 3639 rotates. Including the follower 3645a allows for the driven element 3645 to be slid over the shaft 3639 during assembly. In some embodiments, the groove 3639a is constant along the length of the shaft 3639 so that the driven element 3645 is translated at a constant rate when the shaft 3639 rotates at a constant rate. In some embodiments, the groove 3639a is variable along the length of the shaft 3639 so that the driven element 3645 translates at different rates along the length of the shaft 3639 while the shaft 3639 rotates at a constant rate.

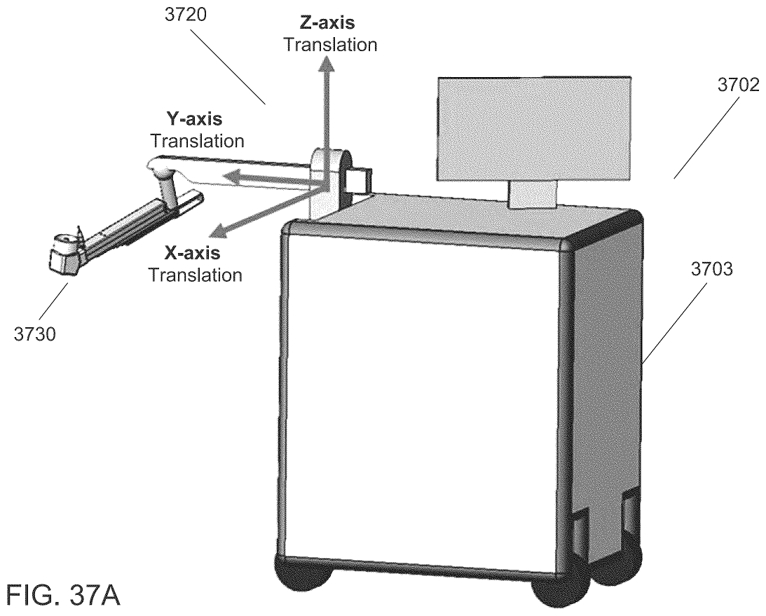

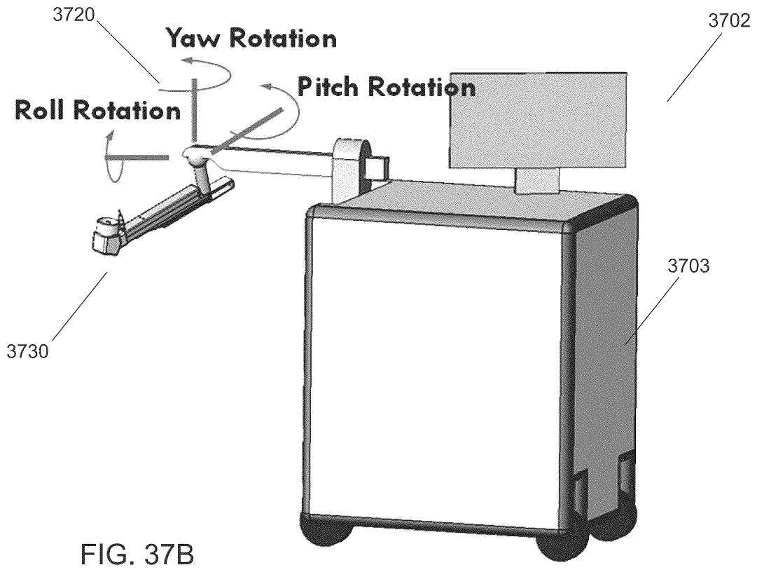

FIG. 37A-37B illustrates another example robotic system 3702 (e.g., structurally and/or functionally similar to the robotic system 2020 of FIG. 2), in accordance with some embodiments. The robotic system 3702 includes a base 3703, a robotic arm 3720, and a manipulation device 2730. The robotic arm 3720 is coupled to the base 3702 such that the robotic arm 3720 can translate along an X-axis, a Y-axis, and a Z-axis as seen in FIG. 37A. In some embodiments, the robotic arm 3720 is coupled to the base 3702 via a rail. The position of the robotic arm 3720 can reduce the hanging weight of the robotic arm 3720 and make it more stable during operation. The robotic arm 3720 is coupled to the manipulation device 3730 via a joint that is configured to provide the manipulation device 3730 with yaw rotation, pitch rotation, and roll rotation.

The translation and rotation of the robotic arm 3720 allows the for the manipulation device 3730 to be positioned in space to a desired position and orientation while holding the manipulation device 3730 stationary. In some embodiments, the robotic arm 3720 can be motorized and controlled by a human interface (e.g., controller, joystick, etc.). In some embodiments, once the robotic arm 3720 positions the manipulation device 3730 in a desired position, the robotic arm 3720 can be locked into place with integrated joint brakes. In some embodiments, information related to operation of the robotic arm 3720 is shown on a display.

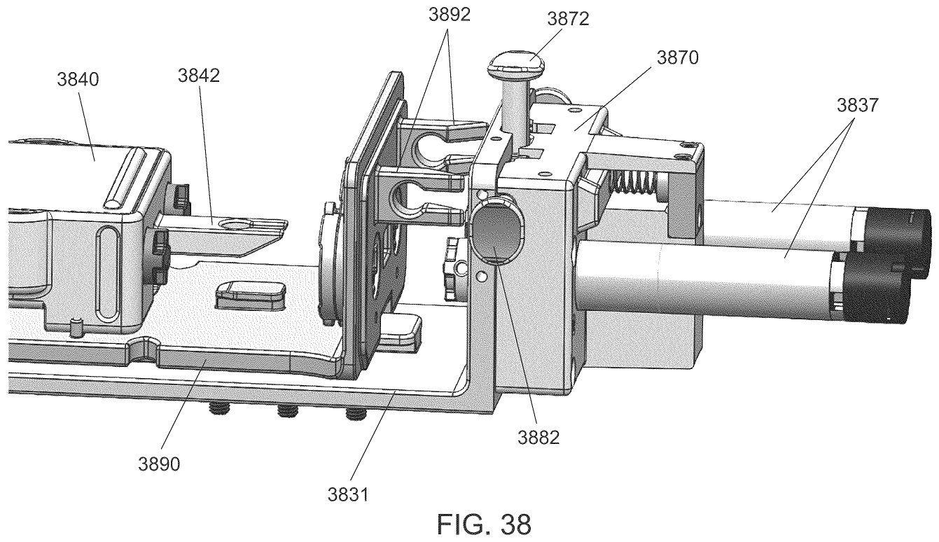

FIGS. 38-41B generally relate to a cartridge (e.g., structurally and/or functionally similar to any of the cartridges described herein, such as the cartridge assembly 540 of FIG. 5) and an adaptor (e.g., functionally and/or structurally similar to any of the adaptors described herein, such as the adaptor 4290 of FIG. 24) including latching mechanism. The latching mechanism allow for the cartridge and/or the adaptor to selectively coupled to motors (e.g., functionally and/or structurally similar to any of the motors described herein, such as the motors 4137a and/or 4137b of FIG. 23) to allow for a sterile barrier to be selectively coupled between the cartridge and the manipulation device (e.g., structurally and/or functionally similar to any of the manipulation devices described herein, such as the manipulation device 130 of FIG. 1). In some embodiments, latching described below can be mechanical, magnetically, and/or the like.

FIG. 38 illustrate a cartridge latch 3842 of a cartridge 3840 and an adaptor latch 3892 of an adaptor 3890, in accordance with some embodiments. FIG. 38 additionally illustrates an interface plate 3831 (e.g., structurally and/or functionally similar to the interface plate 4031 of FIG. 22), a latching assembly 3870 including a cartridge release button 3872, adaptor release buttons 3882, and an adaptor engagement assembly 3880, and motors 3837. The cartridge 3840 and the adaptor 3890 are shown in an unlatched position in FIG. 38.

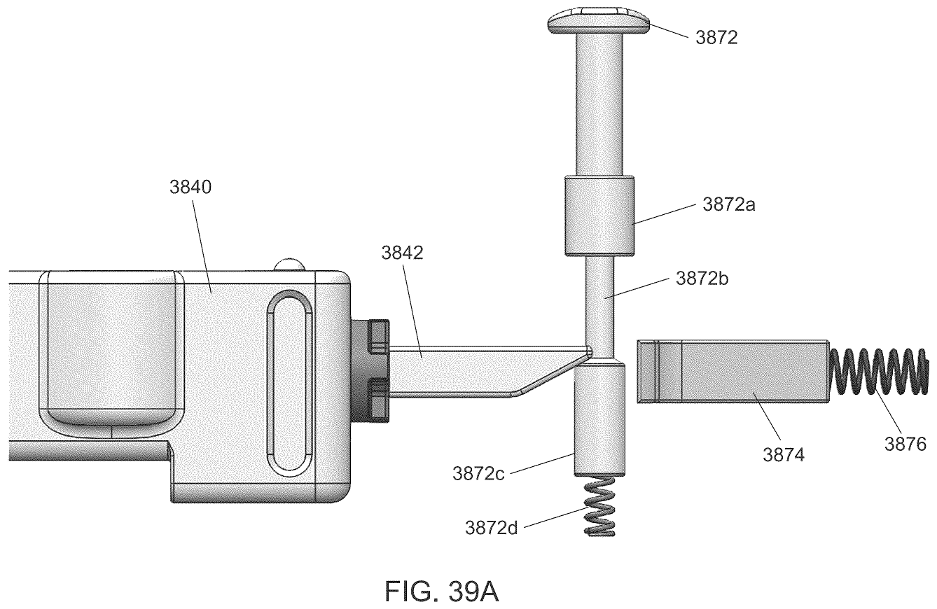

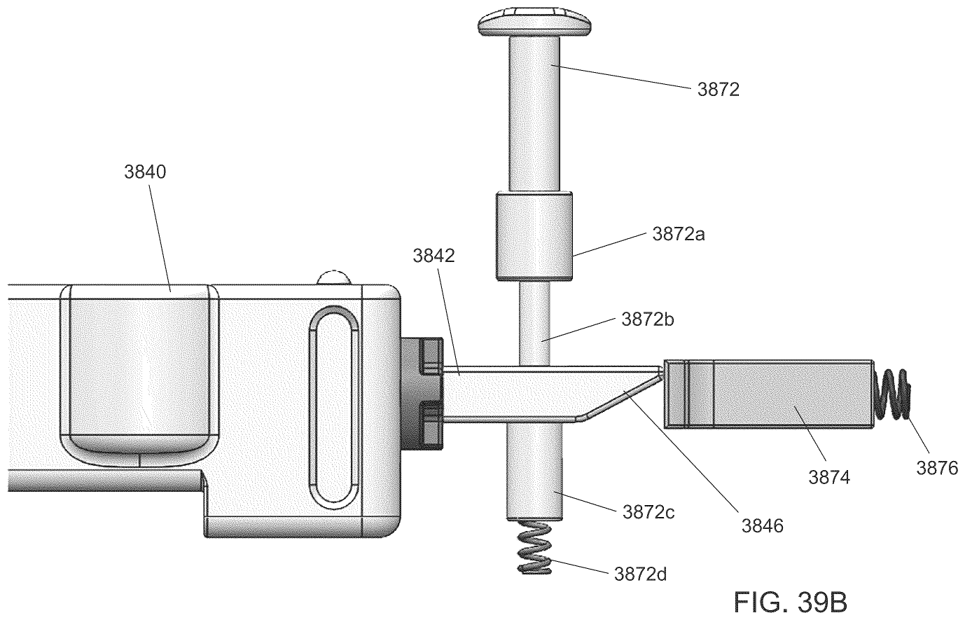

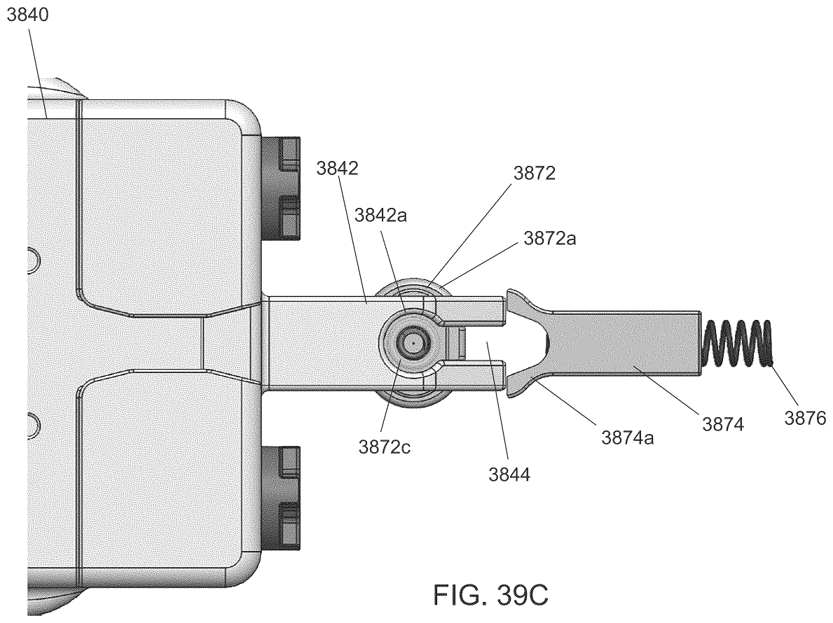

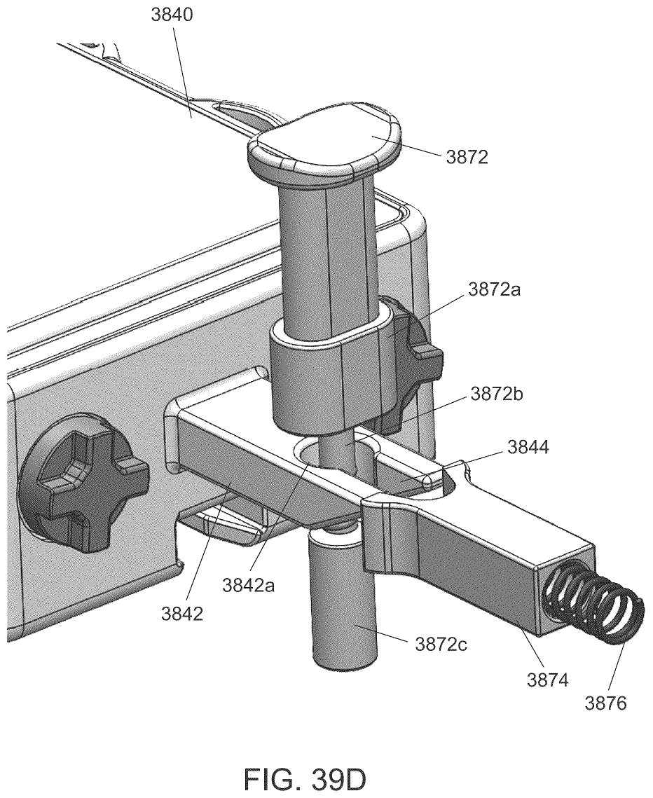

The cartridge latch 3842 extends away from the cartridge 3840 and includes features that allow the cartridge 3840 to selectively couple to the release assembly 3870 via an opening in the adaptor 3890 and an opening in the interface plate 3831. After the cartridge latch 3842 is engaged with the release assembly 3870, the cartridge latch button 3872 can be pressed to release the cartridge 3872. Releasing the cartridge 3840 via the cartridge release button can allow for the cartridge to quickly be removed after a procedure. The operation of the cartridge latch 3842 is described in more detail in reference to FIGS. 39A-39D.

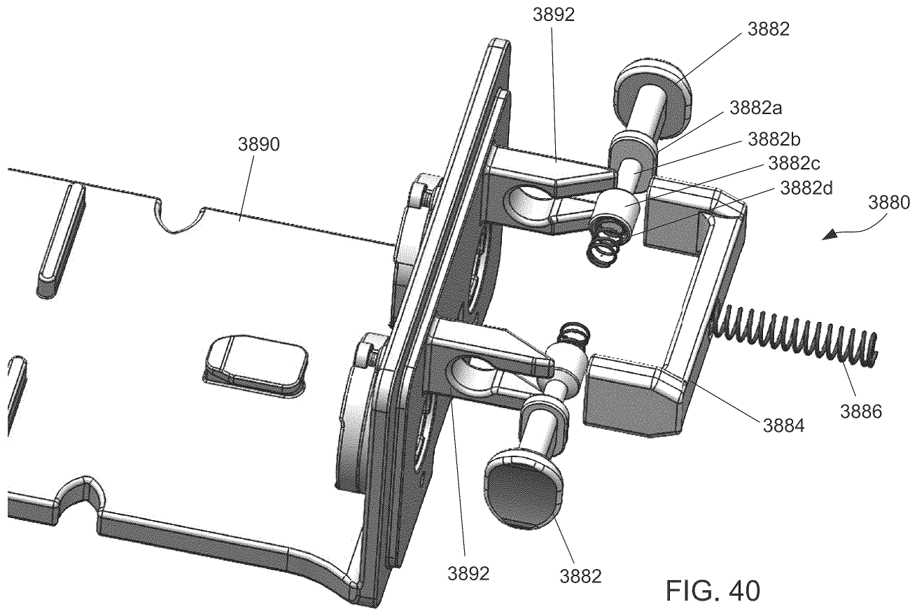

The adaptor latch 3892 includes two latches that extend away from a surface of the adaptor 3890. The adaptor latch 3892 include features that allow for the adaptor 3890 to selectively couple to the release assembly 3870 via openings in the interface plate 3831. After the adaptor latch 3892 is engaged with the release assembly 3870, the adaptor latch buttons 3882 can simultaneously be pressed to release the cartridge 3872. When the cartridge 3840 and the adaptor 3890 are couple to the release assembly 3870, cartridge may be prepared for a procedure. The operation of the cartridge latch 3842 is described in more detail in reference to FIG. 40.

The release assembly 3870 is configured to both hold the cartridge latch 3842 and the adaptor latches 3892 in place when coupled and to eject the cartridge 3840 and the adaptor 3892 when the cartridge release button 3872 and the adapter release buttons 3882 are pressed. The components and the operation of the release assembly 3870 are described in more detail in reference to FIGS. 39A-40.

FIGS. 39A-39D illustrate the cartridge latch 3842 engaging a cartridge latching and release mechanism, in accordance with some embodiments. The cartridge latching and release mechanism includes the cartridge release button 3872, an ejector 3874, and an ejector spring 3876. The cartridge release button 3872 includes a first portion 3872a, a second portion 3872b, a third portion 3872c, and a release button spring 3872d.

As seen in FIG. 39A, which illustrates a side view of the cartridge 3840, the first portion 3872a has a first diameter, the second portion 3872b has a second diameter, and the third portion 3872c includes a third diameter. The first diameter is greater than third diameter which is greater than the second diameter. As seen in FIG. 39D, the first portion 3872a is larger than an opening 3842a in the cartridge latch 3842 so that, when pressed down, the cartridge release button 3872 is prevented from being pressed down more than a predetermined distance and to allow for the cartridge 3840 to disengage when the cartridge release button 3872 is depressed. The second portion 3872b is configured to slide into an opening 3844 of the cartridge latch 3842 so that the cartridge release button 3872 can engage the cartridge latch 3842, as seen in FIGS. 39C-39D. The opening 3844 is sized such that the first portion 3872a and the third portion 3872c are too large to fit through the opening 3844.

The third portion 3872c is configured to engage an angled portion 3846 of the cartridge latch 3842 during engagement of the cartridge release button 3872. The angled portion 3846 allows for the cartridge to be lowered into position when the cartridge 3840 is pushed against the cartridge release button 3872. The third portion 3872c is configured to engage a recessed portion 3842a of the cartridge latch 3842, as seen in FIG. 39C, which illustrates a bottom view of the cartridge 3840 latched to the cartridge release button 3872. When the third portion 3872c is engaged with the recessed portion, the cartridge 3840 is latched, as additionally seen in FIG. 39B. The release button spring 3872d is configured press up against the third portion 3872c so that the third portion 3872c is pressed into the recessed portion of the cartridge latch 3842 when the cartridge 3840 is latched. In some embodiments, the recessed portion 3842a may include a keying feature that is configured to accept a corresponding keying feature on the cartridge release button 3872.

As seen in FIGS. 39B-39C, the ejector 3874 is configured to provide a pressing force (e.g., from the ejector spring 3876) on the cartridge latch 3842 when the cartridge latch 3842 is engaging the cartridge release button 3872. The pressing force from the ejector 3874 pushes, and thus ejects, the cartridge 3840 when the cartridge release button 3872 is pressed. In some embodiments, such as those shown in FIGS. 39B-39D, the ejector 3874 is shaped so that ejector 3874 pushes on the tips of the angled portion 3846.

FIG. 40 illustrates the adaptor 3890 engaging the adaptor latching engagement 3880 with a portion of the latching assembly 3870 removed to show the adaptor engagement mechanism 3880. The adaptor latching mechanism functions similarly to the cartridge latching mechanism. The adaptor latches 3892 are structurally similar to the cartridge latch 3872 and include angled portions that are configured to allow for the adaptor release buttons 3882 to be moved into an engaged position when the adaptor latches 3892 are pushed against the adaptor release buttons 3882. Similar to the cartridge release button 3872, the adaptor release buttons 3882 include a first position 3882a, a second portion 3882b, a third portion 3882c, and a release spring 3882d, which are functionally similar to the first portion 3872a, the second portion 3872b, the third portion 3872c, and the release button spring 3872d, respectively. When the adaptor latches 3892 engage the adaptor release buttons 3882, the cartridge release buttons 3882 move so that the third portion 3882c engages a recessed portion of the adaptor latch 3892 and latching the adaptor 3890 to the adaptor release button 3882.

The adaptor engagement mechanism 3880 further includes an ejector 3884 and an ejector spring 3886. The ejector 3884 functions similarly to the ejector 3874 but includes two portions extending away from a main body. Each of the portions are configured to engage a corresponding adaptor latch 3892. The ejector spring 3886 is configured to provide a pressing force onto the ejector 3884, which then presses on the adaptor latches 3892. When the adaptor release buttons 3882 are pressed, the ejector 3884 pushes, and ejectors, the adaptor 3890 away from the cartridge release buttons 3882. In some embodiments, the engagement of the cartridge latch 3842 and/or the adaptor latch 3892 may include sensors (e.g., hall effect sensor, position sensors, etc.) that can determine the position of the latches 3842, 3892 and/or whether latching was successful. In some embodiments, the signal from the sensors can be used to determine if a procedure may proceed, based on whether latching was successful.

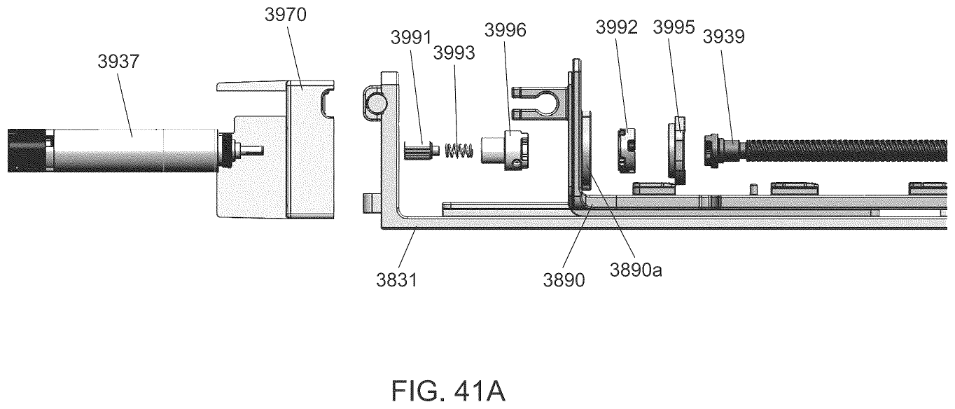

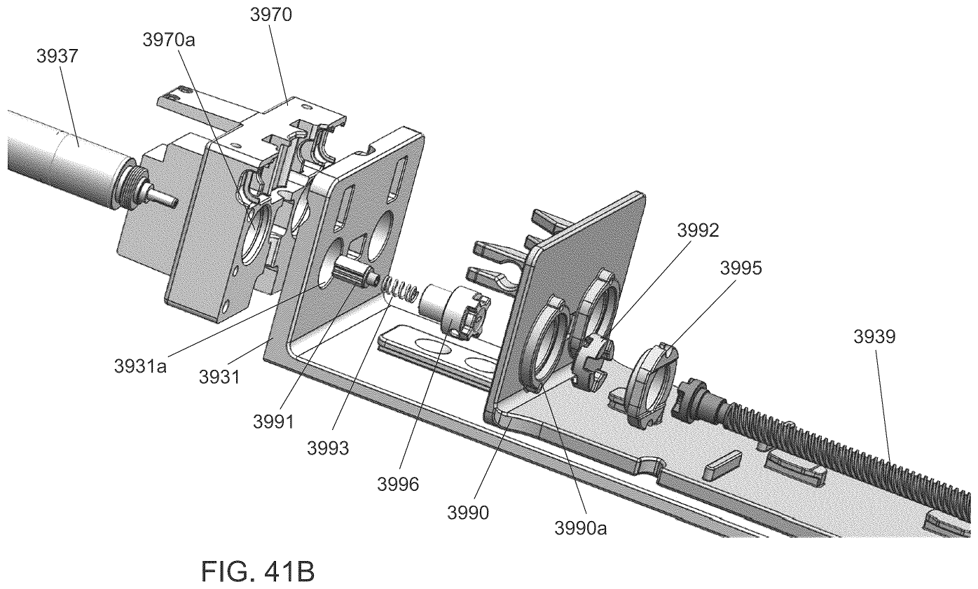

FIGS. 41A-41B depict an exploded view of a clutch system, in accordance with some embodiments. The clutch system is configured to operatively couple a motor 3937 (e.g., structurally and/or functionally similar to any of the motors described herein, such as the motor 3837 of FIG. 38) to a linear actuator 3939 (e.g., structurally and/or functionally similar to actuator(s) 534a, 534c). The clutch system includes a spline 3991 (e.g., functionally and/or structurally similar to the spline 4291 of FIG. 25), a spring 3993 (e.g., functionally and/or structurally similar to the spring 4293 of FIG. 25), a clutch 3996 (e.g., functionally and/or structurally similar to the disposable clutch 4296 of FIG. 25), a puck 3992 (e.g., functionally and/or structurally similar to the puck 4292 of FIG. 25), and a puck cover 3995, arranged linearly from the motor 3937 to the linear actuator 3939. As seen in FIG. 41B, the clutch assembly passes through an opening 3970a of a release assembly 3970 (e.g., structurally and/or functionally similar to the release assembly 3870 of FIG. 38), an opening 3931a of an interface plate 3931 (e.g., functionally and/or structurally similar to the interface plate 3831 of FIG. 38), and an opening 3990a of an adaptor 3990 (e.g., functionally and/or structurally similar to the adaptor 3890 of FIG. 38). In some embodiments, the function of the clutch system may be the same or similar to the clutch system described in reference to FIG. 25.

The spline 3991 operably couples to a motor shaft of the motor 3937. In some embodiments, the spline 3991 may be coupled to the motor shaft via a fastener (e.g., clip, set screw, etc.), mechanically (e.g., slot, friction fit, etc.), welded, or via an adhesive (e.g., glue, epoxy, etc.). The spline 3991 engages and operably couples to a clutch 3996. The spring 3993 between the spline 3991 and the clutch 3996 is housed in a lumen of the clutch 3996 and provides a constant pressure on the clutch 3996 as it engages and operably couples to the puck 3992. The puck and the clutch 3996 include a corresponding extrusion pattern, that, when engaged allow the clutch 3996 to rotate the puck 3992. Similarly, the puck 3992 operably couples to the linear actuator 3939. The puck 3992 and the linear actuator 3939 include a corresponding extrusion pattern, that, when engaged allow the puck 3992 to rotate the linear actuator 3939. The puck cover 3995 covers the puck 3992. In some embodiments, the puck cover 3995 is configured to isolate the puck from the linear actuator 3939 for sanitation purposes.

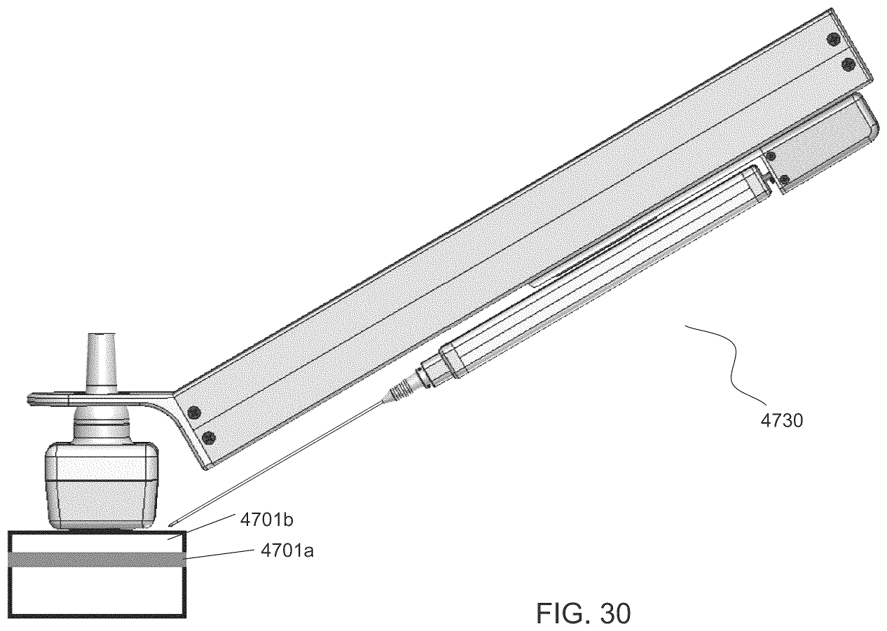

FIG. 30 illustrates the anatomical positioning of the manipulation device 4730 with respect to a blood vessel 4701a, in accordance with some embodiments. As seen in FIG. 30, the manipulation device 4730 is positioned at an angle relative to the arm of a subject, and thereby at an angle relative to the blood vessel. For instance, the manipulation device 4730 can be at any suitable angle (e.g., angle between about 20 degrees to about 60 degrees) relative to the blood vessel. The cartridge of the manipulation device 4730 can include the catheter along with needle and/or guidewire to access the blood vessel based on the angle of the manipulation device 4730 relative to the arm of the subject. The manipulation device 4730 is configured such that the cartridge may insert a catheter past the skin surface 4701b into the blood vessel 4701a, but not deeper than the blood vessel 4701a.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Some embodiments and/or methods described herein can be performed by a different software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

It will be understood that, although the terms first, second, third, etc. may be used herein to describe various elements, these elements should not be limited by these terms. These terms are only used to distinguish one element from another element. Thus, a first element discussed below could be termed a second element without departing from the teachings of the present disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

1. An apparatus, comprising:
a cart movable from a first location to a second location near a patient;
a manipulation device configured to releasably couple to a cartridge including a needle, a catheter, and a guidewire that are coaxially disposed with respect to each other, the manipulation device and the cartridge include a plurality of actuators each configured to couple to a different one of the needle, the catheter, and the guidewire to selectively advance the needle, the catheter, and the guidewire;
an adaptor disposed between the manipulation device and the cartridge, the adaptor configured to provide a sterile barrier between the manipulation device and the cartridge; and
a robotic arm having a first end mounted to the cart and a second end coupled to the manipulation device, the robotic arm having a plurality of segments joined together via a plurality of joints such that the robotic arm can be moved to position the manipulation device.

2. The apparatus of claim 1, further comprising an imaging system coupled to a distal end portion of the manipulation device, the imaging system configured to capture a transverse view and a longitudinal view each including a target vessel.

3. The apparatus of claim 2, wherein the imaging system is configured to change the transverse view as a tip of the needle is advanced into the target vessel to show the transverse view that corresponds to a transverse plane of the tip of the needle.

4. The apparatus of claim 2, wherein the imaging system includes an ultrasound array.

5. The apparatus of claim 1, wherein the cartridge includes at least one linear actuator, the at least one linear actuator configured to advance at least one of the catheter or the guidewire.

6. The apparatus of claim 5, wherein the manipulation device includes at least one motor, the at least one motor configured to be operably coupled to the at least one linear actuator of the cartridge.

7. The apparatus of claim 6, wherein the at least one motor and the at least one linear actuator are coupled via a clutch system.

8. The apparatus of claim 6, wherein the at least one motor is housed in a housing of the manipulation device, wherein the housing and the cartridge are coupled at an interface plate.

9. The apparatus of claim 8, wherein the adaptor is configured to selectively couple to the manipulation device and the cartridge.

10. The apparatus of claim 9, wherein the adaptor is configured to couple to a drape, the drape configured to protect components of the manipulation device from contamination.

11. The apparatus of claim 1, wherein the manipulation device is pivotably supported by a joint of the plurality of joints with respect to a distalmost segment of the robotic arm such that an angle of insertion of the needle, the catheter, and the guidewire into a target vessel can be adjusted via the joint.

12. The apparatus of claim 1, wherein the cartridge is coupled to the manipulation device via a latching system, the latching system including an ejector button configured to eject the cartridge when pressed.

13. The apparatus of claim 1, further comprising:
an imaging system configured to capture a view including at least a part of the manipulation device and a portion of the patient including a target vessel; and
a communication interface configured to send image data of the view to a remote compute device such that a position of the cartridge relative to the portion of the patient can be confirmed by a user at the remote compute device.

14. An apparatus, comprising:
a cartridge including:
a guidewire, a needle, and a catheter that are coaxially disposed with respect to each other; and
a plurality of guides coupled to the guidewire, the needle, and the catheter;
a manipulation device configured to linearly advance and retract the plurality of guides to move the needle, the guidewire, and the catheter;
a plurality of actuators including at least one actuator disposed in the cartridge and at least one actuator disposed in the manipulation device;
at least one motor; and
a clutch system configured to operably couple the at least one motor to the at least one actuator disposed in the cartridge, the clutch system configured to transfer mechanical energy form the at least one motor to the at least one actuator disposed in the cartridge.

15. The apparatus of claim 14, further including a plurality of actuators including:
a first linear actuator configured to linearly advance and retract a first guide of the plurality of guides to move the guidewire;
a second linear actuator configured to linearly advance and retract a second guide of the plurality of guides to move the catheter; and
a third linear actuator configured to linearly advance and retract the cartridge.

16. The apparatus of claim 15, wherein each of the first linear actuator, the second linear actuator, and the third linear actuator includes a plurality of shafts.

17. The apparatus of claim 15, wherein the first linear actuator and the second linear actuator are housed in the cartridge, and the third linear actuator is housed within the manipulation device.

18. The apparatus of claim 14, wherein the clutch system is configured to selectively couple the at least one motor to the at least one actuator.

19. The apparatus of claim 14, wherein the cartridge is configured to store the guidewire in a linear state.

20. The apparatus of claim 14, wherein the clutch system is disposable.

21. The apparatus of claim 14, wherein the clutch system is configured to selectively engage the at least one actuator disposed in the cartridge.

22. The apparatus of claim 14, wherein the clutch system includes a spline, the spline coupled to the at least one motor, and a clutch, the clutch including an extrusion pattern configured to operably couple the clutch to the at least one actuator disposed in the cartridge.

23. The apparatus of claim 22, wherein the clutch system further includes a spring, the spring configured to provide constant pressure to engage the clutch system.

24.-33. (canceled)
